(19) United States
(12) Patent Application Publication
(10) Pub. No.: US 2022/0042035 A1
(43) Pub. Date: Feb. 10, 2022

(54) NON-VIRAL DNA VECTORS AND USES THEREOF FOR ANTIBODY AND FUSION PROTEIN PRODUCTION

(71) Applicant: GENERATION BIO CO., Cambridge, MA (US)

(72) Inventors: Ozan ALKAN, Cambridge, MA (US); Douglas Anthony KERR, Cambridge, MA (US); Robert Michael KOTIN, Cambridge, MA (US); Debra KLATTE, Cambridge, MA (US); Leah LIU, Cambridge, MA (US); Nathaniel SILVER, Cambridge, MA (US)

(21) Appl. No.: 16/968,990

(22) PCT Filed: Feb. 14, 2019
(86) PCT No.: PCT/US2019/018016

§ 371 Date: Aug. 11, 2020

Related U.S. Application Data
(60) Provisional application No. 62/680,092, filed on Jun. 4, 2018.
(60) Provisional application No. 62/680,087, filed on Jun. 4, 2018.
(60) Provisional application No. 62/630,676, filed on Feb. 14, 2018.
(60) Provisional application No. 62/630,670, filed on Feb. 14, 2018.

(51) Int. Cl. C12N 15/85; C12N 15/62; C07K 16/18

(52) U.S. Cl. CPC C12N 15/85; C12N 15/62; A61K 48/00; C12N 2830/50; C07K 16/18

(57) ABSTRACT

The application describes ceDNA vectors having linear and continuous structure for delivery and expression of a transgene. ceDNA vectors comprise an expression cassette flanked by two ITR sequences, where the expression cassette encodes a transgene. Some ceDNA vectors further comprise cis-regulatory elements, including regulatory switches. Further provided herein are methods and cell lines for reliable gene expression in vitro, ex vivo and in vivo using the ceDNA vectors. Provided herein are method and compositions comprising ceDNA vectors useful for the expression of an antibody or fusion protein in a cell, tissue or subject. Such antibodies or fusion proteins can be expressed for treating disease or alternatively, for the production of antibodies or fusion proteins in a commercial setting.

### CROSS REFERENCE TO RELATED APPLICATIONS

This application is a 35 U.S.C. § 371 national stage filing of International Application No. PCT/US2019/018016, filed on Feb. 14, 2019, which in turn claims benefit under 35 U.S.C. § 119(e) of U.S. Provisional Application No. 62/630,670, filed on Feb. 14, 2018, U.S. Provisional Application No. 62/680,087, filed on Jun. 4, 2018, U.S. Provisional Application No. 62/630,676, filed on Feb. 14, 2018, and U.S. Provisional Application No. 62/680,092, filed on Jun. 4, 2018. The contents of each of the aforementioned applications are incorporated herein by reference in their entireties.

### SEQUENCE LISTING

The instant application contains a Sequence Listing which has been submitted electronically in ASCII format and is hereby incorporated by reference in its entirety. Said ASCII copy, created on Feb. 13, 2019, is named 080170-091100-WOPT_SL.txt and is 128,788 bytes in size.

### TECHNICAL FIELD

The present invention relates to the field of gene therapy, including non-viral vectors for expressing a transgene or isolated polynucleotides in a subject or cell. The disclosure also relates to nucleic acid constructs, promoters, vectors, and host cells including the polynucleotides as well as methods of delivering exogenous DNA sequences to a target cell, tissue, organ or organism. For example, the present disclosure provides methods for using non-viral DNA vectors to express antibodies, such as therapeutic antibodies, from a cell. The present disclosure also provides methods for using non-viral DNA vectors to express fusion proteins, such as therapeutic fusion proteins, from a cell. The methods and compositions can be applied to e.g., commercial antibody or fusion protein production or for the purpose of treating disease by expressing a therapeutic antibody or fusion protein in a cell or tissue of a subject in need thereof.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g. underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene), e.g., that can result in a positive gain-of-function effect, a negative loss-of-function effect, or another outcome. Such outcomes can be attributed to expression of an activating antibody or fusion protein or an inhibitory (neutralizing) antibody or fusion protein. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors. Among the many virus-derived vectors available (e.g., recombinant retrovirus, recombinant lentivirus, recombinant adenovirus, and the like), recombinant adeno-associated virus (rAAV) is gaining popularity as a versatile vector in gene therapy.

Adeno-associated viruses (AAV) belong to the parvoviridae family and more specifically constitute the dependoparvovirus genus. Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010), and as a result, use of AAV vectors has been limited to less than 150,000 Da protein coding capacity. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression.

Additionally, conventional AAV virions with capsids are produced by introducing a plasmid or plasmids containing the AAV genome, rep genes, and cap genes (Grimm et al., 1998). However, such encapsidated AAV virus vectors were found to inefficiently transduce certain cell and tissue types and the capsids also induce an immune response.

Accordingly, use of adeno-associated virus (AAV) vectors for gene therapy is limited due to the single administration to patients (owing to the patient immune response), the limited range of transgene genetic material suitable for delivery in AAV vectors due to minimal viral packaging capacity (about 4.5 kb), and slow AAV-mediated gene expression.

There is need in the field for a technology that permits expression of a therapeutic antibody (e.g., a secreted antibody or intrabody) or fusion protein (e.g., a receptor extracellular domain-Fc fusion) in a cell, tissue or subject or, alternatively, for the purpose of generating antibodies or fusion proteins in vitro or in vivo for purification and/or commercial production. In addition, there remains an important unmet need for controllable recombinant DNA vectors with improved production and/or expression properties for the improved production of antibodies (e.g., therapeutic antibodies) and fusion proteins (e.g., therapeutic fusion proteins) compared to existing or conventional methods or vectors.

### BRIEF DESCRIPTION OF THE INVENTION

The technology described herein relates to methods and compositions for expression of antibodies and fusion proteins (such as a therapeutic antibodies and fusion proteins) using a capsid-free (e.g., non-viral) DNA vector with covalently-closed ends (referred to herein as a “closed-ended DNA vector” or a “ceDNA vector”). These ceDNA vector can be used to produce antibodies and fusion proteins for treatment of diseases, treatment of malignancies, monitoring, and diagnosis, as well as for commercial antibody or fusion protein production. One exemplary antibody is an anti-Tumor Necrosis Factor antibody or antibody-binding fragment thereof, including but not limited to a monoclonal antibody adalimumab (Humira™), which can be expressed in a cell or tissue of a subject using the ceDNA vectors described herein. Such a therapeutic antibody can be used for the purpose of treating rheumatoid arthritis, psoriatic arthritis, ankylosing spondylitis and Crohn's disease.

Accordingly, the invention described herein relates to a capsid-free (e.g., non-viral) DNA vector with covalently-closed ends (referred to herein as a “closed-ended DNA vector” or a “ceDNA vector”) comprising a heterogeneous gene encoding an antibody (e.g., light chains, heavy chains, frameworks, Fabs′, single clain antibodies) or antigen-binding fragment thereof, to permit expression of an antibody within a cell, for example, a secreted antibody or an intrabody. The invention also relates to a ceDNA vector comprising a heterogeneous gene encoding a fusion protein, to permit expression of a fusion protein within a cell. Such antibodies or fusion proteins to be expressed can be therapeutic antibodies or fusion proteins and/or the techniques applied can be used in the generation of antibodies or fusion proteins for commercial purposes. In particular, the technology described herein relates to the improved production of antibodies and fusion proteins using ceDNA vectors.

The ceDNA vectors for antibody and fusion protein production as described herein are capsid-free, linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence, where the 5′ ITR and the 3′ ITR can have the same symmetrical three-dimensional organization with respect to each other, (i.e., symmetrical or substantially symmetrical), or alternatively, the 5′ ITR and the 3′ ITR can have different three-dimensional organization with respect to each other (i.e., asymmetrical ITRs). In addition, the ITRs can be from the same or different serotypes. In some embodiments, a ceDNA vector can comprise ITR sequences that have a symmetrical three-dimensional spatial organization such that their structure is the same shape in geometrical space, or have the same A, C-C′ and B-B′ loops in 3D space (i.e., they are the same or are mirror images with respect to each other). In some embodiments, one ITR can be from one AAV serotype, and the other ITR can be from a different AAV serotype.

Accordingly, some aspects of the technology described herein relate to a ceDNA vector for improved antibody or fusion protein expression and/or production that comprise ITR sequences selected from any of: (i) at least one WT ITR and at least one modified AAV inverted terminal repeat (ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization. The ceDNA vectors disclosed herein can be produced in eukaryotic cells, thus devoid of prokaryotic DNA modifications and bacterial endotoxin contamination in insect cells.

The methods and compositions described herein relate, in part, to the discovery of a non-viral capsid-free DNA vector with covalently-closed ends (ceDNA vectors) that can be used to express at least one antibody and/or fusion protein, or more than one antibody and/or fusion protein from a cell. The methods and compositions can be applied to e.g., commercial antibody or fusion protein production or for the purpose of treating disease with a therapeutic antibody or fusion protein.

Accordingly, provided herein in one aspect are DNA vectors (e.g., ceDNA vectors) comprising at least one heterologous nucleic acid sequence encoding at least one transgene encoding an antibody or antigen-binding fragment or fusion proteins thereof operably linked to a promoter positioned between two different AAV inverted terminal repeat sequences (ITRs), one of the ITRS comprising a functional AAV terminal resolution site and a Rep binding site, and one of the ITRs comprising a deletion, insertion, or substitution relative to the other ITR; wherein the transgene is an antibody or fragment thereof (e.g., an antigen-binding fragment thereof) or fusion protein; and wherein the DNA when digested with a restriction enzyme having a single recognition site on the DNA vector has the presence of characteristic bands of linear and continuous DNA as compared to linear and non-continuous DNA controls when analyzed on a non-denaturing gel. Other aspects include delivery of a therapeutic antibody or fusion protein by expressing it in vivo from a ceDNA vector as described herein and further, the treatment of a variety of diseases using such antibodies or fusion proteins. Also contemplated herein are cells comprising a ceDNA vector as described herein.

Aspects of the invention relate to methods to produce the ceDNA vectors useful for antibody or fusion protein production or antibody or fusion protein expression in a cell as described herein. Other embodiments relate to a ceDNA vector produced by the method provided herein. In one embodiment, the capsid free (e.g., non-viral) DNA vector (ceDNA vector) for antibody or fusion protein production is obtained from a plasmid (referred to herein as a “ceDNA-plasmid”) comprising a polynucleotide expression construct template comprising in this order: a first 5′ inverted terminal repeat (e.g. AAV ITR); a heterologous nucleic acid sequence; and a 3′ ITR (e.g. AAV ITR), where the 5′ ITR and 3′ITR can be asymmetric relative to each other, or symmetric (e.g., WT-ITRs or modified symmetric ITRs) as defined herein.

The ceDNA vector for antibody or fusion protein production as disclosed herein is obtainable by a number of means that would be known to the ordinarily skilled artisan after reading this disclosure. For example, a polynucleotide expression construct template used for generating the ceDNA vectors of the present invention can be a ceDNA-plasmid, a ceDNA-bacmid, and/or a ceDNA-baculovirus. In one embodiment, the ceDNA-plasmid comprises a restriction cloning site (e.g. SEQ ID NO: 123 and/or 124) operably positioned between the ITRs where an expression cassette comprising e.g., a promoter operatively linked to a transgene, e.g., a nucleic acid encoding an antibody or antigen binding fragment thereof or fusion protein and/or a reporter gene) can be inserted. In some embodiments, ceDNA vectors for antibody or fusion protein production are produced from a polynucleotide template (e.g., ceDNA-plasmid, ceDNA-bacmid, ceDNA-baculovirus) containing symmetric or asymmetric ITRs (modified or WT ITRs).

In a permissive host cell, in the presence of e.g., Rep, the polynucleotide template having at least two ITRs replicates to produce ceDNA vectors for antibody or fusion protein production. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector. Rep proteins and Rep binding sites of the various AAV serotypes are well known to those of ordinary skill in the art. One of ordinary skill understands to choose a Rep protein from a serotype that binds to and replicates the nucleic acid sequence based upon at least one functional ITR. For example, if the replication competent ITR is from AAV serotype 2, the corresponding Rep would be from an AAV serotype that works with that serotype such as AAV2 ITR with AAV2 or AAV4 Rep but not AAV5 Rep, which does not. Upon replication, the covalently-closed ended ceDNA vector continues to accumulate in permissive cells and ceDNA vector is preferably sufficiently stable over time in the presence of Rep protein under standard replication conditions, e.g. to accumulate in an amount that is at least 1 pg/cell, preferably at least 2 pg/cell, preferably at least 3 pg/cell, more preferably at least 4 pg/cell, even more preferably at least 5 pg/cell.

Accordingly, one aspect of the invention relates to a process of producing a ceDNA vector for antibody or fusion protein production comprising the steps of: a) incubating a population of host cells (e.g. insect cells) harboring the polynucleotide expression construct template (e.g., a ceDNA-plasmid, a ceDNA-bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector for antibody or fusion protein production in a host cell. However, no viral particles (e.g. AAV virions) are expressed. Thus, there is no virion-enforced size limitation.

The presence of the ceDNA vector useful for antibody or fusion protein production is isolated from the host cells can be confirmed by digesting DNA isolated from the host cell with a restriction enzyme having a single recognition site on the ceDNA vector and analyzing the digested DNA material on denaturing and non-denaturing gels to confirm the presence of characteristic bands of linear and continuous DNA as compared to linear and non-continuous DNA.

For the purposes of this disclosure, the transgene expressed by the ceDNA encodes an antibody or antibody binding fragment or fusion protein. Antibodies and fusion proteins are well known in the art and can bind to any protein of interest, including, but not limited to, a ligand, a receptor, a toxin, a hormone, an enzyme, or a cell surface protein, or pathogen or viral protein or antigen, as well as pre- and post-translationally modified proteins, such as glycoproteins or SUMOylated proteins (e.g., ant-SUMO2/3 antibody) etc. Antibodies and antigen binding fragments also include antibodies that bind to any antigen, including but not limited to nucleic acids, e.g., DNA (e.g., anti-dsDNA antibodies), RNA (e.g., anti-RNA binding antibodies). In some embodiments, the antibodies produced by the ceDNA vectors disclosed herein are neutralizing antibodies or antigen-binding fragments thereof. Exemplary genes to be targeted and proteins of interest are described in detail in the methods of use and methods of treatment sections herein.

Also provided herein are methods of expressing an antibody or fusion protein that has therapeutic uses, using a ceDNA vector in a cell or subject. Such antibodies or fusion proteins can be used for the treatment of disease. Accordingly, provided herein are methods for the treatment of disease comprising administering a ceDNA vector encoding a therapeutic antibody or fusion protein to a subject in need thereof. In other embodiments the therapeutic antibody or fusion protein can be used to target malignant cells, monitor specific proteins, or for diagnostic purposes.

In some embodiments, the present application may be defined in any of the following paragraphs:

1. A capsid-free close-ended DNA (ceDNA) vector comprising:

at least one heterologous nucleotide sequence between flanking inverted terminal repeats (ITRs), wherein at least one heterologous nucleotide sequence encodes at least one antibody and/or fusion protein.

1. The ceDNA vector of claim 1, wherein at least one heterologous nucleotide sequence encodes an antibody.
2. The ceDNA vector of claim 2, wherein the antibody is a full-length antibody, a Fab, a Fab′, a single-domain antibody, or a single-chain antibody (scFv).
3. The ceDNA vector of claim 3, wherein at least one heterologous nucleotide sequence encodes a single-domain antibody or a single-chain antibody.
4. The ceDNA vector of claim 4, wherein the at least one heterologous nucleotide sequence further encodes a secretory leader sequence upstream of the single-domain antibody or single-chain antibody.
5. The ceDNA vector of any one of claims 1-3, wherein a first heterologous nucleotide sequence encodes a heavy chain variable region and a second heterologous nucleotide sequence encodes a light chain variable region.
6. The cDNA vector of claim 4, wherein the first heterologous nucleotide sequence encodes a heavy chain variable region and a heavy chain constant region or portion thereof, and the second heterologous nucleotide sequence encodes a light chain variable region and a light chain constant region or portion thereof.
7. The ceDNA vector of claim 6 or claim 7, wherein the first heterologous nucleotide sequence and/or the second heterologous nucleotide sequence further encodes a secretory leader sequence upstream of the heavy chain variable region and/or light chain variable region.
8. The ceDNA vector of any one of claims 1-8, wherein the antibody is a human or humanized antibody.
9. The ceDNA vector of any one of claims 1-9, wherein the antibody is an IgG, IgA, IgD, IgM, or IgE antibody.
10. The ceDNA vector of claim 10, wherein the antibody is an IgG antibody.
11. The ceDNA vector of claim 11, wherein the IgG antibody is an IgG1, IgG2, IgG3, or IgG4 antibody.
12. The ceDNA vector of any one of claims 1-12, wherein the antibody binds to at least one target selected from the targets listed in Tables 1, 2, 3A, 3B, 4, and 5.
13. The ceDNA vector of claim 1, wherein at least one heterologous nucleotide sequence encodes a fusion protein.
14. The ceDNA vector of claim 14, wherein the at least one heterologous nucleotide sequence further encodes a secretory leader sequence upstream of the fusion protein.
15. The ceDNA vector of claim 14 or claim 15, wherein the fusion protein comprises at least one receptor extracellular domain fused to an Fc region.
16. The ceDNA vector of claim 16, wherein the receptor extracellular domain is an extracellular domain of a receptor selected from CTLA-4, VEGFR1, VEGFR2, LFA-3, TNFR, IL-1R1, IL-1R1, IL-1RAcP, and ACVR2A.
17. The ceDNA vector of any one of claims 1-17, wherein the antibody or fusion protein is selected from the antibodies and fusion proteins of Tables 1, 2, 3A, 3B, 4, or 5.
18. The ceDNA vector of any one of claims 1-18, wherein the ceDNA vector comprises one or more poly-A sites.
19. The ceDNA vector of any one of claims 1-19, wherein the ceDNA vector comprises at least one promoter operably linked to at least one heterologous nucleotide sequence.
20. The ceDNA vector of any one of claims 1-20, wherein at least one heterologous nucleotide sequence is cDNA.
21. The ceDNA vector of any one of claims 1-21, wherein at least one ITR comprises a functional terminal resolution site and a Rep binding site.
22. The ceDNA vector of any one of claims 1-22, wherein one or both of the ITRs are from a virus selected from a parvovirus, a dependovirus, and an adeno-associated virus (AAV).
23. The ceDNA vector of any one of claims 1-23, wherein the flanking ITRs are symmetric or asymmetric.
24. The ceDNA vector of claim 24, wherein the flanking ITRs are symmetrical or substantially symmetrical.
25. The ceDNA vector of claim 24, wherein the flanking ITRs are asymmetric.
26. The ceDNA vector of any one of claims 1-26, wherein one or both of the ITRs are wild type, or wherein both of the ITRs are wild-type.
27. The ceDNA vector of any one of claims 1-27, wherein the flanking ITRs are from different viral serotypes.
28. The ceDNA vector of any one of claims 1-28, wherein the flanking ITRs are from a pair of viral serotypes shown in Table 6.
29. The ceDNA vector of any one of claims 1-29, wherein one or both of the ITRs comprises a sequence selected from the sequences in Table 7.
30. The ceDNA vector of any one of claims 1-30, wherein at least one of the ITRs is altered from a wild-type AAV ITR sequence by a deletion, addition, or substitution that affects the overall three-dimensional conformation of the ITR.
31. The ceDNA vector of any one of claims 1-31, wherein one or both of the ITRs are derived from an AAV serotype selected from AAV1, AAV2, AAV3, AAV4, AAV5, AAV6, AAV7, AAV8, AAV9, AAV10, AAV11, and AAV12.
32. The ceDNA vector of any one of claims 1-32, wherein one or both of the ITRs are synthetic.
33. The ceDNA vector of any one of claims 1-33, wherein one or both of the ITRs is not a wild type ITR, or wherein both of the ITRs are not wild-type.
34. The ceDNA vector of any one of claims 1-34, wherein one or both of the ITRs is modified by a deletion, insertion, and/or substitution in at least one of the ITR regions selected from A, A′, B, B′, C, C′, D, and D′.
35. The ceDNA vector of claim 35, wherein the deletion, insertion, and/or substitution results in the deletion of all or part of a stem-loop structure normally formed by the A, A′, B, B′ C, or C′ regions.
36. The ceDNA vector of any one of claims 1-36, wherein one or both of the ITRs are modified by a deletion, insertion, and/or substitution that results in the deletion of all or part of a stem-loop structure normally formed by the B and B′ regions.
37. The ceDNA vector of any one of claims 1-37, wherein one or both of the ITRs are modified by a deletion, insertion, and/or substitution that results in the deletion of all or part of a stem-loop structure normally formed by the C and C′ regions.
38. The ceDNA vector of any one of claims 1-38, wherein one or both of the ITRs are modified by a deletion, insertion, and/or substitution that results in the deletion of part of a stem-loop structure normally formed by the B and B′ regions and/or part of a stem-loop structure normally formed by the C and C′ regions.
39. The ceDNA vector of any one of claims 1-39, wherein one or both of the ITRs comprise a single stem-loop structure in the region that normally comprises a first stem-loop structure formed by the B and B′ regions and a second stem-loop structure formed by the C and C′ regions.
40. The ceDNA vector of any one of claims 1-40, wherein one or both of the ITRs comprise a single stem and two loops in the region that normally comprises a first stem-loop structure formed by the B and B′ regions and a second stem-loop structure formed by the C and C′ regions.
41. The ceDNA vector of any one of claims 1-41, wherein one or both of the ITRs comprise a single stem and a single loop in the region that normally comprises a first stem-loop structure formed by the B and B′ regions and a second stem-loop structure formed by the C and C′ regions.
42. The ceDNA vector of any one of claims 1-42, wherein both ITRs are altered in a manner that results in an overall three-dimensional symmetry when the ITRs are inverted relative to each other.
43. The ceDNA vector of any one of claims 1-43, wherein one or both of the ITRs comprises a sequence selected from the sequences in Tables 7, 9A, 9B, and 10.
44. The ceDNA vector of any one of claims 1-44, wherein at least one heterologous nucleotide sequence is under the control of at least one regulatory switch.
45. The ceDNA vector of claim 45, wherein at least one regulatory switch is selected from a binary regulatory switch, a small molecule regulatory switch, a passcode regulatory switch, a nucleic acid-based regulatory switch, a post-transcriptional regulatory switch, a radiation-controlled or ultrasound controlled regulatory switch, a hypoxia-mediated regulatory switch, an inflammatory response regulatory switch, a shear-activated regulatory switch, and a kill switch.
46. A method of expressing an antibody or fusion protein in a cell comprising contacting the cell with the ceDNA vector of any one of claims 1-46.
47. The method of claim 47, wherein the cell contacted is a eukaryotic cell.
48. The method of claim 47 or claim 48, wherein the cell is in vitro or in vivo.
49. The method of any one of claims 47-49, wherein the at least one heterologous nucleotide sequence codon optimized for expression in the eukaryotic cell.
50. The method of any one of claims 47-50, wherein the antibody or fusion protein is secreted from the cell.
51. The method of any one of claims 47-50, wherein the antibody or fusion protein is retained in the cell.
52. A method of treating a subject with a therapeutic antibody or therapeutic fusion protein, comprising administering to the subject a ceDNA vector of any one of claims 1-46, wherein at least one heterologous nucleotide sequence encodes the therapeutic antibody or therapeutic fusion protein.
53. The method of claim 53, wherein the subject has a disease or disorder selected from cancer, autoimmune disease, a neurodegenerative disorder, hypercholesterolemia, acute organ rejection, multiple sclerosis, post-menopausal osteoporosis, skin conditions, asthma, or hemophilia.
54. The method of claim 53, wherein the cancer is selected from a solid tumor, soft tissue sarcoma, lymphoma, and leukemia.
55. The method of claim 53, wherein the autoimmune disease is selected from rheumatoid arthritis and Crohn's disease.
56. The method of claim 53, wherein the skin condition is selected from psoriasis and atopic dermatitis.
57. The method of claim 53, wherein the neurodegenerative disorder is Alzheimer's disease.
58. A pharmaceutical composition comprising the ceDNA vector of any one of claims 1-46.
59. A cell containing a ceDNA vector of any of claims 1-46.
60. A composition comprising a ceDNA vector of any of claims 1-46 and a lipid.
61. The composition of claim 61, wherein the lipid is a lipid nanoparticle (LNP).
62. A kit comprising the ceDNA vector of any one of claims 1-46 or the composition of claim 61 or 62 or the cell of claim 60.
63. A method of producing an antibody or fusion protein comprising culturing the cell of claim 60 under conditions suitable for producing the antibody or fusion protein.
64. The method of claim 64, further comprising isolating the antibody or fusion protein.

In some embodiments, one aspect of the technology described herein relates to a non-viral capsid-free DNA vector with covalently-closed ends (ceDNA vector), wherein the ceDNA vector comprises at least one heterologous nucleotide sequence, operably positioned between two inverted terminal repeat sequences where the ITR sequences can be asymmetric, or symmetric, or substantially symmetrical as these terms are defined herein, wherein at least one of the ITRs comprises a functional terminal resolution site and a Rep binding site, and optionally the heterologous nucleic acid sequence encodes a transgene (e.g., an antibody or fusion protein) and wherein the vector is not in a viral capsid.

These and other aspects of the invention are described in further detail below.

### DESCRIPTION OF DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A illustrates an exemplary structure of a ceDNA vector for antibody or fusion protein production as disclosed herein, comprising asymmetric ITRs. In this embodiment, the exemplary ceDNA vector comprises an expression cassette containing CAG promoter, WPRE, and BGHpA. An open reading frame (ORF) encoding a transgene, e.g., a nucleic acid encoding an antibody or fusion protein can be inserted into the cloning site (R3/R4) between the CAG promoter and WPRE. The expression cassette is flanked by two inverted terminal repeats (ITRs)—the wild-type AAV2 ITR on the upstream (5′-end) and the modified ITR on the downstream (3′-end) of the expression cassette, therefore the two ITRs flanking the expression cassette are asymmetric with respect to each other.

FIG. 1B illustrates an exemplary structure of a ceDNA vector for antibody or fusion protein production as disclosed herein comprising asymmetric ITRs with an expression cassette containing CAG promoter, WPRE, and BGHpA. An open reading frame (ORF) encoding a transgene, e.g., a nucleic acid encoding an antibody or fusion protein can be inserted into the cloning site between CAG promoter and WPRE. The expression cassette is flanked by two inverted terminal repeats (ITRs)—a modified ITR on the upstream (5′-end) and a wild-type ITR on the downstream (3′-end) of the expression cassette.

FIG. 1C illustrates an exemplary structure of a ceDNA vector for antibody or fusion protein production as disclosed herein comprising asymmetric ITRs, with an expression cassette containing an enhancer/promoter, a transgene, a post transcriptional element (WPRE), and a polyA signal. An open reading frame (ORF) allows insertion of a transgene, e.g., a nucleic acid encoding an antibody or fusion protein, into the cloning site between CAG promoter and WPRE. The expression cassette is flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifications).

FIG. 1D illustrates an exemplary structure of a ceDNA vector for antibody or fusion protein production as disclosed herein, comprising symmetric modified ITRs, or substantially symmetrical modified ITRs as defined herein, with an expression cassette containing CAG promoter, WPRE, and BGHpA. An open reading frame (ORF) encoding a transgene, e.g., a nucleic acid encoding an antibody or fusion protein, is inserted into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1E illustrates an exemplary structure of a ceDNA vector for antibody or fusion protein production as disclosed herein comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene, e.g., a nucleic acid encoding an antibody or fusion protein, into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1F illustrates an exemplary structure of a ceDNA vector for antibody or fusion protein production as disclosed herein, comprising symmetric WT-ITRs, or substantially symmetrical WT-TTRs as defined herein, with an expression cassette containing CAG promoter, WPRE, and BGHpA. An open reading frame (ORF) encoding a transgene, e.g., a nucleic acid encoding an antibody or fusion protein, is inserted into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 1G illustrates an exemplary structure of a ceDNA vector for antibody or fusion protein production as disclosed herein, comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene, e.g., a nucleic acid encoding an antibody or fusion protein, into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 2A provides the T-shaped stem-loop structure of a wild-type left ITR of AAV2 (SEQ ID NO: 52) with identification of A-A′ arm, B-B′ arm, C-C′ arm, two Rep binding sites (RBE and RBE′) and also shows the terminal resolution site (trs). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 2B shows proposed Rep-catalyzed nicking and ligating activities in a wild-type left ITR (SEQ ID NO: 53), including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR (SEQ ID NO: 54). FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left) (SEQ ID NO: 113). FIG. 3C shows the primary structure (left) and the secondary structure (right) of the RBE-containing portion of the A-A′ loop, and the B-B′ and C-C′ arms of wild type right AAV2 ITR (SEQ ID NO: 55). FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right) (SEQ ID NO: 114). Any combination of left and right ITR (e.g., AAV2 ITRs or other viral serotype or synthetic ITRs) can be used as taught herein. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of a ceDNA vector for antibody or fusion protein production as disclosed herein in the process described in the schematic in FIG. 4B. FIG. 4B is a schematic of an exemplary method of ceDNA production and FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions). FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as 1 kb and 2 kb in neutral conditions, but in denaturing conditions, the stands remain connected and produce single strands that migrate as 2 kb and 4 kb.

FIG. 5 is an exemplary picture of a denaturing gel running examples of ceDNA vectors with (+) or without (−) digestion with endonucleases (EcoRI for ceDNA construct 1 and 2; BamH1 for ceDNA construct 3 and 4; SpeI for ceDNA construct 5 and 6; and XhoI for ceDNA construct 7 and 8) Constructs 1-8 are described in Example 1 of International Application PCT PCT/US18/49996, which is incorporated herein in its entirety by reference. Sizes of bands highlighted with an asterisk were determined and provided on the bottom of the picture.

FIGS. 6A-6C show exemplary constructs and plasmids for generating a ceDNA vector for antibody or fusion protein production, and shows for exemplary purposes a ceDNA vector encoding aducanumab. One of ordinary skill can readily replace the nucleic acid encoding aducanumab with any other nucleic acid encoding a different antibody or fusion protein. FIG. 6A shows an exemplary ceDNA plasmid (pFBdual-ceDNA-aducanumab; SEQ ID NO: 56) for generating an aducanumab (full IgG1) expressing ceDNA vector. This ceDNA plasmid comprises the nucleic acid sequence for expressing aducanumab that has been codon optimized, flanked between an asymmetric ITR pair (i.e., a WT 5′ ITR (wt ITR) and a 3′ mod-ITR (R-asym ITR). This ITR pair can be easily replaced by another asymmetric ITR-pair or symmetric ITR pair as described herein. Moreover, this plasmid comprises, flanked between the ITR-pair and in a 5′ to 3′ direction: a SV40 enhancer (SEQ ID NO: 126), a human EF alpha promoter (SEQ ID NO: 77) or fragment thereof (SEQ ID NO: 78), and VH1-02 secretory leader sequence (SEQ ID NO: 88), an optimized aducanumab heavy chain (HC) nucleic acid sequence (SEQ ID NO: 57), a SV40 polyA sequence (SEQ ID NO: 86), and upstream of the aducanumab light chain (LC) sequence the following: a CMV enhancer (SEQ ID NO: 83), a rEFI promoter (SEQ ID NO: 85 or SEQ ID NO: 150), a VK A26 leader sequence (SEQ ID NO: 89), an optimized aducanumab light chain (LC) nucleic acid sequence (SEQ ID NO: 58) and BGH polyadenylation sequence (SEQ ID NO: 68 or SEQ ID NO: 148). The optimized aducanumab heavy chain (HC) sequence and optimized aducanumab light chain (LC) nucleic acid sequence can be readily substituted for any other heavy chain or light chain sequences of the antibodies described herein, e.g., see Tables 1-5 herein. FIG. 6B is an exemplary insert that can be used as a modular component to be inserted into a desired ceDNA vector to generate a plasmid as in FIG. 6A. FIG. 6C is a linearized view of a region of the ceDNA-Adu-full-IgG1 plasmid comprising the sequences for generating aducanumab.

FIGS. 7A-7G show exemplary ceDNA vectors that can express a variety of different antibodies or antigen-binding fragments or fusion proteins as disclosed herein. The ceDNA vectors exemplified also illustrate multiple configurations with respect to the use of IRES sequences, promoter sequences, enhancer sequences, linker sequences, polyadenylation sequences. FIG. 7A shows an embodiment of a ceDNA vector construct for producing antibodies with a polyA sequence after the heavy chain sequence and an optional enhancer upstream of the light chain nucleic acid sequence. FIG. 7B shows an embodiment of a ceDNA vector construct for producing antibodies with a polyA sequence after the heavy chain sequence and an IRES upstream of the light chain nucleic acid sequence. FIG. 7C shows an embodiment of a ceDNA vector construct for producing antibody fragments (e.g., antigen binding fragments) similar to FIG. 7A, including with a polyA sequence after the heavy chain Fab Fragment sequence and an optional enhancer upstream of the light chain fragment nucleic acid sequence. FIG. 7D shows an embodiment of a ceDNA vector construct for producing an antibody as disclosed herein, including with a polyA sequence after the light chain sequence. FIG. 7E shows an embodiment of a ceDNA vector construct for producing an antibody as disclosed herein, including with a polyA sequence after the heavy chain sequence. FIG. 7F shows an embodiment of a ceDNA vector construct for producing a single domain antibody (dAb) as disclosed herein, including with a polyA sequence after the dAb sequence. FIG. 7G shows an embodiment of a ceDNA vector construct for producing an antibody fragment, such as a single chain variable fragment fusion protein (scFv) or single chain antibody as disclosed herein, including with a polyA sequence after scFv sequence of single chain antibody sequence. As one of skill in the art will appreciate, the ceDNA vectors for antibody production as described herein can be used in a modular fashion, such that desired regulatory sequences or heterologous nucleic acids encoding an antibody or fragment thereof can be interchanged with other desired sequences. That is, the ceDNA vectors are customizable for a desired application. Also shown in FIGS. 7A-7G are embodiments where the nucleic acid sequences for the variable chains (V_{H} and V_{L}) and constant chains (C_{H} and C_{L}), and Fc sequences are located proximal to each other, or alternatively the Fc can be joined to a sequence encoding V_{H} and V_{L} via a linker sequence as disclosed herein.

FIGS. 8A-8B show exemplary SDS-Page (FIG. 8A) and Western blot (FIG. 8B) analysis of the expression of the aducanumab (full IgG1) antibody expressed from the ceDNA-IgG1-Adu construct as described in Example 9 after a one step purification of the expressed protein. FIG. 8A shows SDS-PAGE gel image of the expressed antibody. The lanes are as follows: M1 is a protein marker (Takara cat. no. 3452), The), and the purified aducanumab is shown in reducing conditions (Lane 1) and non-reducing (Lane 2) conditions. The presence of two bands in the reducing and only a single band in the non-reducing conditions is consistent with the protein being an antibody with heavy and light chains which migrates as a single band in nonreducing conditions and as the constituent heavy and light chains under reducing conditions. FIG. 8B shows a Western blot image immunostained with an anti-human IgG antibody. The lanes are as follows: M2 is a protein marker (GenScript, cat. no. M00521), and P is a positive control human IgG1 antibody (Sigma).

FIG. 9A-9B shows expression of ceDNA expressing GFP or aducanumab (full IgG1) antibody expressed from the ceDNA-IgG1-Adu vector. FIG. 9A provides fluorescent microscopic images of HEK293T cells transfected with ceDNA-GFP plasmid (upper panel) and ceDNA-GFP vector (lower panel), as described in Example 8. The presence of abundant fluorescence in both images show that significant transfection and expression of the transgene GFP occurred in cells with either ceDNA treatment. FIG. 9B provides two different images of the same membrane transfer of cellular samples separated electrophoretically by SDS-PAGE, as described in Example 8. The bottom panel is the Ponceau stained membrane showing all protein content; the top panel is a Western blot where the visible bands reflect the presence of human antibody. In lanes 7-10 the antibody heavy chain migrates at approximately 50 kDa and the antibody light chain migrates at approximately 25 kDa; both chains are visible in all four lanes.

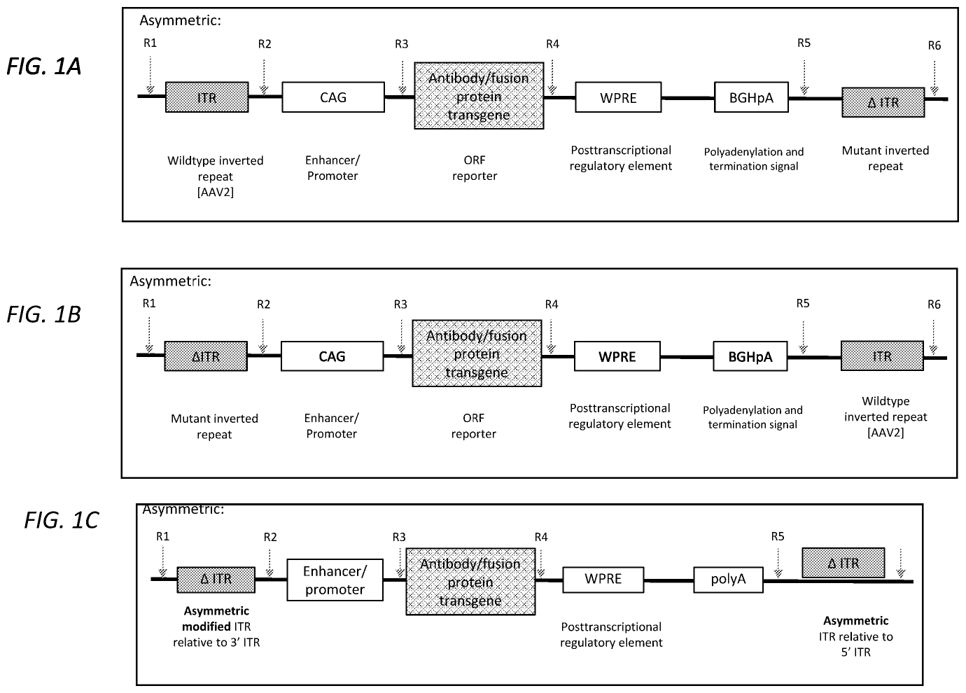

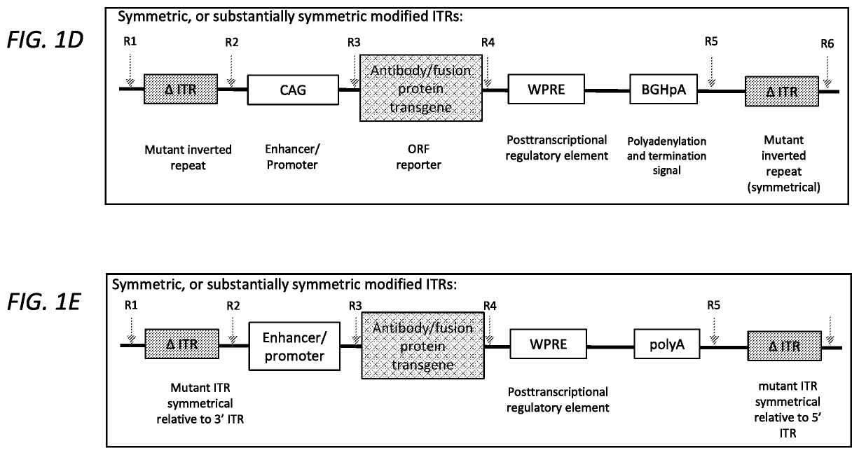

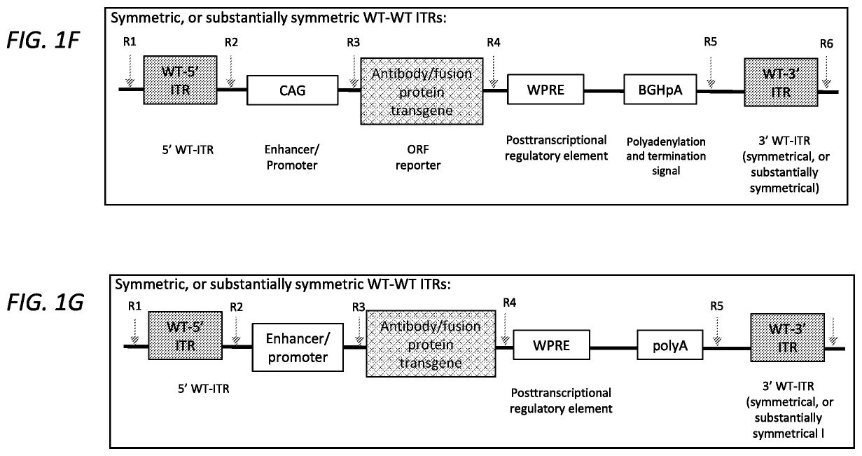

FIG. 1A-10B show characterization of the ceDNA produced aducanumab antibody. FIG. 10A shows the results of the HPLC analysis described in Example 9, showing a single peak corresponding to the ceDNA-produced aducanumab. FIG. 10B depicts the results of an ELISA analysis assessing the ability of the purified aducanumab antibody to recognize immobilized beta-amyloid (1-42) ligand, as described in Example 9.

FIG. 11 graphically depicts the results of the experiments described in Example 10. The negative control samples from mice treated with ceDNA constructs lacking aducanumab transgenes (labelled as ceDNA negative control) were at or below the lower limit of quantification in the assay. In contrast, the serum of mice treated with the ceDNA-IgG construct had high levels of human immunoglobulin present at both the day 3 and day 7 timepoints.

FIG. 12 provides two different time exposures of the same membrane transfer of cellular samples separated electrophoretically by SDS-PAGE, as described in Example 12. The top panel was taken at a 6 second exposure and the lower panel was taken after a 20 second exposure. Bands corresponding to the intact antibody are seen at the top of the gel (and a limited amount of reduced heavy and light chains migrating at ˜50 kDa and ˜25 kDa, respectively) are visible in lanes 5, 7 (both aducanumab), and 11 (bevacizumab) (see arrows). In lane 9, the presence of the Fc fusion protein is observed near the top of the lane, and no lower molecular weight constituent products are observed, as expected.

### DETAILED DESCRIPTION OF THE INVENTION

Provided herein are ceDNA vectors for antibody production as described herein comprising one or more heterologous nucleic acids that encode for an antibody (e.g., heavy chains, light chains, framework, Fab′, scAb). Provided herein are also ceDNA vectors for fusion protein production as described herein comprising one or more heterologous nucleic acids that encode for a fusion protein. Such vectors can be used in commercial antibody or fusion protein production or in the delivery of a therapeutic antibody or fusion protein as described herein, by intracellular expression from the ceDNA vector. In some embodiments, the expression of the antibody or fusion protein can comprise secretion of the antibody or fusion protein out of the cell in which it is expressed or alternatively in some embodiments, the expressed antibody or fusion protein can target a protein within the cell in which it is expressed (e.g., the antibody is an intrabody). In some embodiments, the ceDNA vector expresses an antibody or antigen-binding fragment thereof or fusion protein in a muscle (e.g., skeletal muscle) of a subject, which can act as a depot for antibody or fusion protein production and secretion to many systemic compartments.

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “antibody” encompasses any antibody or antibody fragment (i.e., a functional antibody fragment), or antigen-binding fragment that retains antigen-binding activity to a desired antigen or epitope. In one embodiment, the antibody or antigen-binding fragment thereof comprises an immunoglobulin chain or fragment thereof and at least one immunoglobulin variable domain sequence. Examples of antibodies include, but are not limited to, an scFv, a Fab fragment, a Fab′, a F(ab′)₂, a single domain antibody (dAb), a heavy chain, a light chain, a heavy and light chain, a full antibody (e.g., includes each of the Fc, Fab, heavy chains, light chains, variable regions etc.), a bispecific antibody, a diabody, a linear antibody, a single chain antibody, an intrabody, a monoclonal antibody, a chimeric antibody, or multimeric antibody. In addition, an antibody can be derived from any mammal, for example, primates, humans, rats, mice, horses, goats etc. In one embodiment, the antibody is human or humanized. In some embodiments, the antibody is a modified antibody. In some embodiments, the components of an antibody can be expressed separately such that the antibody self-assembles following expression of the protein components. In some embodiments, the antibody has a desired function, for example, interaction and inhibition of a desired protein for the purpose of treating a disease or a symptom of a disease. In one embodiment, the antibody or antigen-binding fragment thereof comprises a framework region or an F_{c} region. An antibody fragment can retain 10-99% of the activity of the complete antibody (e.g., 10-90%, 10-80%, 10-70%, 10-60%, 10-50%, 10-40%, 10-30%, 10-20%, 50-99%, 50-90%, 50-80%, 50-70%, 50-60%, 20-99%, 30-99%, 40-99%, 60-99%, 70-99%, 80-99% 90-99% or any activity therebetween). It is also contemplated herein that the functional antibody fragment comprises an activity that is greater than the activity of the intact antibody (e.g., at least 2-fold or higher). In another embodiment, the antibody fragment comprises an affinity for its target that is substantially similar to the affinity of the intact antibody for the same target (e.g., epitope). The antibody can be “activating” such that it increases the activity of a target protein, or “inhibitory” (e.g., a neutralizing or blocking antibody) such that it decreases activity of the target protein.

As used herein, the term “antigen-binding domain” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3). The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.” For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). Each VH and VL typically includes three CDRs and four FRs, arranged from amino-terminus to carboxy-terminus in the following order: FR1, CDR1, FR2, CDR2, FR3, CDR3, FR4.

As used herein, the term “full length antibody” refers to an immunoglobulin (Ig) molecule (e.g., an IgG, IgE, IgM antibody), for example, that is naturally occurring, and formed by normal immunoglobulin gene fragment recombinatorial processes.

As used herein, the term “functional antibody fragment” or “antigen-binding fragment” are used interchangeably and refer to an antibody fragment that binds to the same antigen or epitope as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. In some embodiments, the functional antibody fragment retains at least 20% of the activity of the intact or full-length antibody, for example, as assessed by measuring the degree of activation or inhibition of the target protein. In other embodiments, the functional antibody fragment retains at least 30%, at least 40%, at least 50%, at least 60%, at least 70%, at least 80%, at least 90%, at least 95%, at least 98%, at least 99%, or even 100% (i.e., substantially similar) activity to the intact antibody. It is also contemplated herein that a functional antibody fragment will comprise increased activity as compared to the intact antibody (e.g., at least 1-fold, at least 2-fold, at least 5-fold, at least 10-fold, at least 100-fold or more).

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

A DNA sequence that “encodes” a particular antibody or antigen-binding fragment is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “fusion protein” as used herein refers to a polypeptide which comprises protein domains from at least two different proteins. For example, a fusion protein may comprise (i) an antibody or fragment thereof (e.g., an antigen-binding portion or antigen-binding fragment of an antibody) or a ligand binding domain and (ii) at least one non-antibody protein. Fusion proteins encompassed herein include, but are not limited to, an antibody, or Fc or antigen-binding fragment of an antibody fused to a protein of interest, e.g., an extracellular domain of a receptor, ligand, enzyme or peptide. The antibody or antigen-binding fragment thereof that is part of a fusion protein can be a monospecific antibody or a bispecific or multispecific antibody.

As used herein, the term “genomic safe harbor gene” or “safe harbor gene” refers to a gene or loci that a nucleic acid sequence can be inserted such that the sequence can integrate and function in a predictable manner (e.g., express a protein of interest) without significant negative consequences to endogenous gene activity, or the promotion of cancer. In some embodiments, a safe harbor gene is also a loci or gene where an inserted nucleic acid sequence can be expressed efficiently and at higher levels than a non-safe harbor site.

As used herein, the term “gene delivery” means a process by which foreign DNA is transferred to host cells for applications of gene therapy.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-TTR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g. AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 60), an RBS sequence identified in AAV2. Any known RBS sequence may be used in the embodiments of the invention, including other known AAV RBS sequences and other naturally known or synthetic RBS sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 60). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 61), the hexanucleotide sequence identified in AAV2. Any known TRS sequence may be used in the embodiments of the invention, including other known AAV TRS sequences and other naturally known or synthetic TRS sequences such as AGT (SEQ ID NO: 62), GGTGG (SEQ ID NO: 63), AGTGG (SEQ ID NO: 64), AGTGA (SEQ ID NO: 65), and other motifs such as RRTRR (SEQ ID NO: 66).

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes a heterologous DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present invention, is provided. Usually the animal is a vertebrate such as, but not limited to a primate, rodent, domestic animal or game animal. Primates include but are not limited to, chimpanzees, cynomologous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment−Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous,” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleotide sequence encoding a fusion variant polypeptide.

A “vector” or “expression vector” is a replicon, such as plasmid, bacmid, phage, virus, virion, or cosmid, to which another DNA segment, i.e. an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. Expression of an Antibody or Fusion Protein from a ceDNA Vector

The technology described herein is directed in general to the expression and/or production of an antibody or fusion protein in a cell from a non-viral DNA vector, e.g., a ceDNA vector as described herein. ceDNA vectors for antibody or fusion protein production are described herein in the section entitled “ceDNA vectors in general”. In particular, ceDNA vectors for antibody or fusion protein production comprise a pair of ITRs (e.g., symmetric or assymetric as described herein) and between the ITR pair, a nucleic acid encoding an antibody or fusion protein, as described herein, operatively linked to a promoter or regulatory sequence. A distinct advantage of ceDNA vectors for antibody or fusion protein production over traditional AAV vectors, and even lentiviral vectors, is that there is no size constraint for the heterologous nucleic acid sequences encoding a desired protein. Thus, even a full length antibody comprising e.g., two heavy chain Fc regions, a linker, and a Fab fragment can be expressed from a single ceDNA vector. For the expression and/or production of antibodies in particular, given their size the ceDNA vector may be advantageous over traditional vectors because of the ease of use and the lack of size constraint makes it readily possible to express antibodies (including multimeric antibodies) with different domain structures in a controlled fashion. In addition, multiple administrations can be made permitting cocktails of different ceDNA vectors expressing different antibodies or fusion proteins to be administered. Thus, the ceDNA vectors described herein can be used to express a therapeutic antibody or fusion protein in a subject in need thereof. Alternatively, the ceDNA vectors can be used in the production of antibodies or fusion proteins in a commercial setting, for example, using a bioreactor or for production in a desired host.

As one will appreciate, the ceDNA vector technologies described herein can be adapted to any level of complexity or can be used in a modular fashion, where expression of different components of an antibody or fusion protein can be controlled in an independent manner. For example, it is specifically contemplated that the ceDNA vector technologies designed herein can be as simple as using a single ceDNA vector to express a single heterologous gene sequence (e.g., a heavy chain or a light chain of a desired antibody) or can be as complex as using multiple ceDNA vectors, where each vector expresses multiple antibody or antibody components that are each independently controlled by different promoters. The following embodiments are specifically contemplated herein and can adapted by one of skill in the art as desired.

In on embodiment, a single ceDNA vector can be used to express a single component of an antibody or fusion protein, for example, a heavy chain or light chain. Alternatively, a single ceDNA vector can be used to express multiple components (e.g., at least 2) of an antibody or fusion protein under the control of a single promoter (e.g., a strong promoter), optionally using an IRES sequence(s) to ensure appropriate expression of each of the components.

Also contemplated herein, in another embodiment, is a single ceDNA vector comprising at least two inserts (e.g., expressing a heavy chain or light chain), where the expression of each insert is under the control of its own promoter. The promoters can include multiple copies of the same promoter, multiple different promoters, or any combination thereof. As one of skill in the art will appreciate, it is often desirable to express components of an antibody at different expression levels, thus controlling the stoichiometry of the individual components expressed to ensure efficient antibody folding and combination in the cell.

Additional variations of ceDNA vector technologies can be envisioned by one of skill in the art or can be adapted from antibody production methods using conventional vectors.

A. Heterogeneous Sequences for Expressing an Antibody or Fusion Protein

Essentially any antibody or antigen-binding fragment (e.g., functional fragment) thereof or fusion protein can be expressed from a ceDNA vector as described herein. One of skill in the art will understand that an antibody fragment comprises, at a minimum, the amino acids necessary for antigen or epitope binding (e.g., scAb, Fab, F(ab′)₂, dAb, and Fv). For example, an antibody molecule can include a heavy (H) chain variable domain sequence (abbreviated herein as VH), and a light (L) chain variable domain sequence (abbreviated herein as VL). In one embodiment, an antibody molecule comprises or consists of a heavy chain and a light chain (referred to herein as a half antibody). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. Such functional antibody fragments retain the ability to selectively bind with their respective antigen or epitope, and to activate or inhibit the target protein.

Also encompassed herein are ceDNA vectors that express fusion proteins. In some embodiments, a fusion protein is a biofunctional fusion protein. In some embodiments, a fusion protein is useful for trap technology, e.g., antibody-ligand traps, where the fusion protein comprises an antibody (e.g., monospecific or bispecific antibody) or antibody fragment (e.g., an antigen-binding fragment) fused to a peptide, or a ligand binding domain or receptor domain that traps a ligand, thereby inhibiting the activity of the ligand. Accordingly, in some embodiments the ceDNA vector can encode and express a fusion protein that is referred to in the art as a trap or Y-trap. In some embodiments, a ceDNA vector as described herein is used to express a fusion protein, for example, a VEGF-Trap fusion protein, a IGF-trap fusion protein (see Vaniotis et al., Sci Rep, 2018, 8(1), 17361) or a TGFβ-Trap fusion protein. An exemplary TGFβ-Trap is a Y-trap, e.g., a bifunctional antibody-ligand trap (Y-trap) comprising an antibody targeting CTLA4 and/or PD-L1 fused to a TGFβ receptor II ectodomain sequence that simultaneously disables autocrine/paracrine TGFβ in the target cell microenvironment (a-CTLA4-TGFβRIIecd and a-PDL1-TGFβRIIecd) (see, e.g., Ravi et al., Nat Comm 2018, 9(1); 741). In some embodiments, a fusion protein encompassed for use herein and expressed by the ceDNA vector is an antibody-ligand trap, where the fusion protein comprises an antibody or antibody fragment (e.g., an antigen-binding fragment) fused to a ligand binding domain or receptor domain (e.g., extracellular receptor domain) that traps a ligand, where the ligand is selected from any commonly known growth factors or ligands, including but not limited to, IGF, VEGF, TGFβ, TNFα, EGF, NGF, PDGF, LFA-3, CTLA-4, IL-1, TPO.

Exemplary fusion proteins include, but are not limited to, Etanercept (ENBREL®), which comprises a 75 kDa soluble extracellular domain (ECD) of tumor necrosis factor (TNF) receptor II fused to human IgG1 Fc; Alefacept (AMEVIVE®) which comprises the first ECD of lymphocyte function-associated antigen 3 (LFA-3) fused to human IgG1 Fc; Abatacept (ORENCIA®) which comprises an ECD of human cytotoxic T lymphocyte associated molecule-4 (CTLA-4) fused to human IgG1 Fc; Rilonacept (ARCALYST®) which comprises two chains, each comprising the C-terminus of the IL-1R accessory protein ligand binding region fused to the N-terminus of the IL-1RI ECD, fused to human IgG1 Fc; Romiplostim (NPLATE®) which comprises a peptide thrombopoietin (TPO) mimetic fused to the C-terminus of aglycosylated human IgG1 Fc; Belatacept (NULOJIX®) which comprises an ECD of CTLA-4 fused to human IgG1 Fc and differs from abatacept by two amino acid substitutions (L104E, A29Y) in the CTLA-4 region.

Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody can be monoclonal. An antibody produced using the methods described herein can be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein. By inserting the coding sequences for such antibodies into a ceDNA vector virtually any antibody can be produced. In one embodiment, the light chain and heavy chain genes are under the control of a regulatory switch. In the same or alternative embodiments, the light and heavy chain genes are connected with an IRES sequence (e.g., SEQ ID NO: 190).

Typically, the antibody or fusion protein gene will also encode a secretory sequence so that the antibody is directed to the Golgi Apparatus and Endoplasmic Reticulum whence the antibody will be folded into the correct conformation by chaperone molecules as it passes through the ER and out of the cell. Exemplary secretory sequences include, but are not limited to VH-02 (SEQ ID NO: 88) abd VK-A26 (SEQ ID NO: 89) and Igκ signal sequence (SEQ ID NO: 126), as well as a Gluc secretory signal that allows the tagged protein to be secreted out of the cytosol (SEQ ID NO: 188), TMD-ST secretory sequence, that directs the tagged protein to the golgi (SEQ ID NO: 189).

When an intrabody is desired, the nucleic acid or gene encoding the antibody typically does not code a secretory sequence. In some instances, it can encode a secretory sequence but also has an intended targetting sequence, such as, but not limited to, a KDEL sequence to keep it within the cell. In other embodiments, the intrabody genes encode another intracellular targeting sequence, e.g., a nuclear localization sequence.

Regulatory switches can be used to fine tune the expression of the antibodies (including intrabodies) or fusion proteins so that the antibody is expressed as desired, including but not limited to expression of the antibody or fusion protein at a desired expression level or amount, or alternatively, when there is the presence or absence of particular signal, including a cellular signaling event. For instance, as described herein, expression of the antibody or fusion protein from the ceDNA vector can be turned on or turned off when a particular condition occurs, as described herein in the section entitled Regulatory Switches.

For example, and for illustration purposes only, the antibodies or fusion proteins can be used to turn off an undesired reaction as with anti-TNFα antibodies, such as adalimumab. In other cases, the antibody or fusion protein can help augment an immune reaction. For example, with respect to a malignant cell, e.g., a tumor. The antibody gene can contain a tumor-associated marker to bring the antibody to the desired cell. However, in either situation it can be desirable to regulate the expression of the antibody or fusion protein. ceDNA vectors readily accommodate the use of regulatory switches with the antibodies. The ceDNA vectors also permit control of the stoichiometry of the heavy and light chains. Examples of fusion proteins include, but are not limited to, VEGF-trap and TGFβ-trap technologies.

Antibody molecules include intact molecules as well as antigen-binding fragments and functional fragments thereof. Constant regions of the antibody molecules can be altered, e.g., mutated, to modify the properties of the antibody (e.g., to increase or decrease one or more of: Fc receptor binding, the number of cysteine residues etc.). Examples of antigen-binding fragments of an antibody molecule include, but are not limited to: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using the ceDNA vectors and can be screened for utility in the same manner as are intact antibodies.

A distinct advantage of ceDNA vectors over traditional AAV vectors, and even lentiviral vectors, is that there is no size constraint for the heterologous nucleic acid sequences encoding a desired protein. Thus, even a full length antibody comprising a two heavy chain Fc regions, a linker, and a Fab fragment can be expressed from a single ceDNA vector. In addition, depending on the necessary stoichemistry one can express multiple segments of the same antibody or fusion protein e.g., light chain and heavy chain, and can use same or different promoters, and can also use regulatory switches to fine tune expression of each region. For example, as shown in the Examples, a ceDNA vector that comprises a dual promoter system can be used, so that a different promoter is used for each of the heavy chain and light chains of the aducanumab antibody. Use of a ceDNA plasmid to produce an antibody or fusion protein can include a unique combination of promoters for expression of the heavy and light chain that results in the proper ratios of heavy and light chains for the formation of functional antibody or fusion protein. Accordingly, in some embodiments, a ceDNA vector can be used to express different regions of an antibody or fusion protein separately (e.g., under control of a different promoter). In some embodiments, the nucleic acid encoding the heavy chain can be operatively linked to a first promoter or first regulatory switch and the nucleic acid encoding the light chain can be can be operatively linked to a second promoter or second regulatory switch, thus enabling controlled or tunable expression of the heavy chain and the light chain, independent of one another, enabling control of the ratios of the heavy chain and light chain for production of a functional antibody or fusion protein.

Expression of an antibody or fusion protein from a ceDNA vector can be achieved both spatially and temporally using one or more inducible or repressible promoters.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine.

In some embodiments, the antibody is a multispecific antibody, which comprises two or more variable regions to bind to at least two different epitopes, for example, on the same target protein, or to simultaneously target at least two different proteins. That is, the epitopes recognized by the multispecific antibody can be on the same or different targets.

In other embodiments, the antibody is a bispecific antibody, which can recognize and bind to at least two different epitopes or targets (e.g., see e.g., Riethmuller, G Cancer Immunity (2012) 12:12-18; Schaefer w et al. PNAS (2011) 108(27):11187-92 for exemplary bispecific antibody structures). Second generation bispecific antibodies, for example, “trifunctional bispecific” antibodies are also contemplated with the methods and compositions described herein.

In certain embodiments, an antibody provided is a multispecific antibody, including a bispecific antibody. Multispecific antibodies are monoclonal antibodies that have binding specificities for at least two different sites. An exemplary bispecific antibody is one where one of the binding specificities is for Abeta and the other is for any other antigen. In certain embodiments, bispecific antibodies may bind to two different epitopes of Abeta. Bispecific antibodies may also be used to localize cytotoxic agents to cells. Bispecific antibodies can be prepared as full length antibodies or antibody fragments.

In some embodiments, a ceDNA vector for producing a multispecific antibody comprises the co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)): using leucine zippers to produce bi-specific antibodies (see, e.g., Kostelny et al., J. Immunol., 148(5):1547-1553 (1992)); using “diabody” technology for making bispecific antibody fragments (see. e.g., Hollinger et al., Proc. Natl. Acad. Sci. USA. 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

In some embodiments, the ceDNA vector encodes an engineered antibody with three or more functional antigen binding sites, including “Octopus antibodies,” are also included herein (see. e.g. US 2006/0025576A1). In some embodiments, a ceDNA vector encodes an antibody or fusion protein which is a “Dual Acting FAb” or “DAF” comprising an antigen binding site that binds to Abeta as well as another, different antigen (see, US 2008/0069820, for example).

In one embodiment, the antibody is an “antibody variant,” which refers to an antibody having an altered amino acid sequence, composition or structure as compared to its corresponding native antibody. For example, the antibody variant can comprise a non-native secretion signal to permit the antibody to be secreted from the host cell.

In certain embodiments, a ceDNA vector encodes a cysteine engineered antibody variant, e.g., “thioMAbs,” where one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In another embodiment, the antibody can be a miniaturized antibody, which are monovalent or bivalent antibodies comprising a variable light chain, a variable heavy antigen binding domain and, optionally, one or more effector domains (e.g., tissue-specific targeting). Although the use of miniaturized antibodies is specifically contemplated herein, the ceDNA vectors are not constrained by size with respect to heterologous nucleic acid sequences and therefore have the advantage of expressing even a full-length antibody.

In another embodiment, the antibody or fusion protein expressed from the ceDNA vectors further comprises an additional functionality, such as fluorescence, enzyme activity, secretion signal or immune cell activator.

In some embodiments, the antibody encoded by the ceDNA vector comprises a diabody (bispecific single-chain antibodies) or unibodies (IgG4 molecule lacking a hinge region to reduce the risk of immune activation).

CeDNA vectors as described herein for antibody production are also useful in expression of fusion proteins or intrabodies (i.e., intracellular antibodies) that can target intracellular proteins that affect cell function (e.g., metabolism, cell division, transcription, translation etc.). An intrabody can be an scFv. The intrabodies can be directed to a particular cellular compartment by incorporating signaling motifs, such as a C-terminal ER retention signal (e.g., KDEL), a mitochondrial targeting sequence, a nuclear localization sequence, etc.

Intrabodies are particularly well suited for treatment of diseases associated with misfolded proteins, for example, Alzheimer's disease, Parkinson's disease, prion disease, Huntington's disease etc.

In some embodiments, the antibody or fusion protein can further comprise a linker domain, for example. As used herein “linker domain” refers to an oligo- or polypeptide region from about 2 to 100 amino acids in length, which links together any of the domains/regions of the antibody as described herein. In some embodiment, linkers can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. The linker can be a linker region is T2A derived from Thosea asigna virus.

It is well within the abilities of one of skill in the art to take a known and/or publically available protein sequence of e.g., a fusion protein, heavy chain, light chain, variable region etc., and reverse engineer a cDNA sequence to encode such a protein (e.g., fusion protein) or antibody. The cDNA can then be codon optimized to match the intended host cell and inserted into a ceDNA vector as described herein.

In one embodiment, the antibody-encoding sequence can be derived from an existing hybridoma cell line, for example, by reverse transcribing mRNA obtained from a hybridoma cell line and amplifying the sequence using PCR.

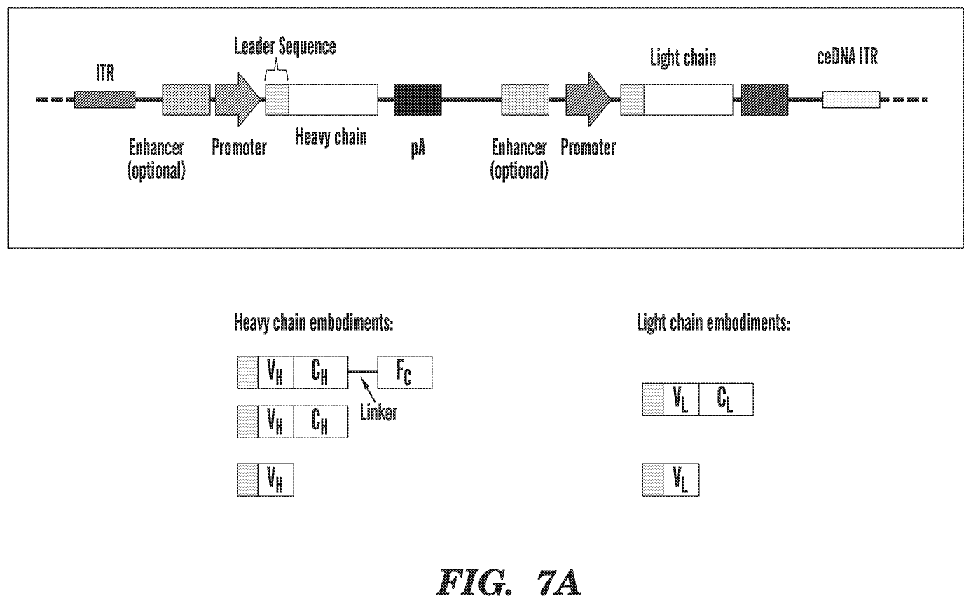

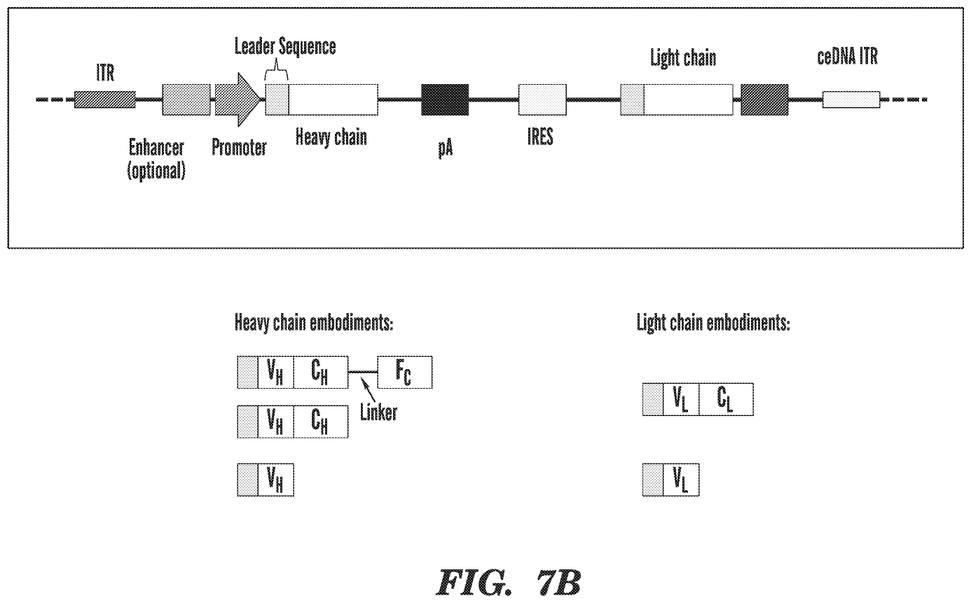

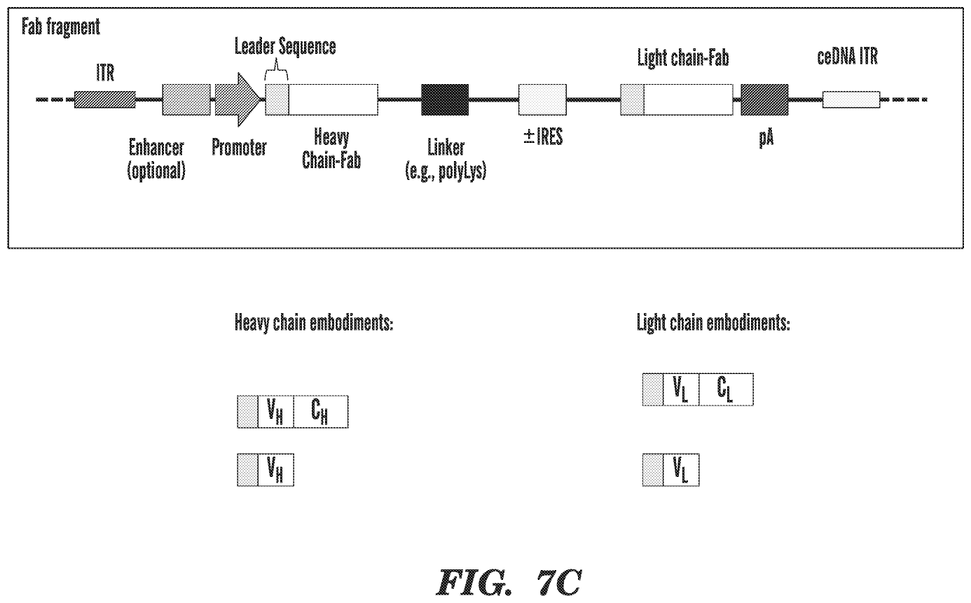

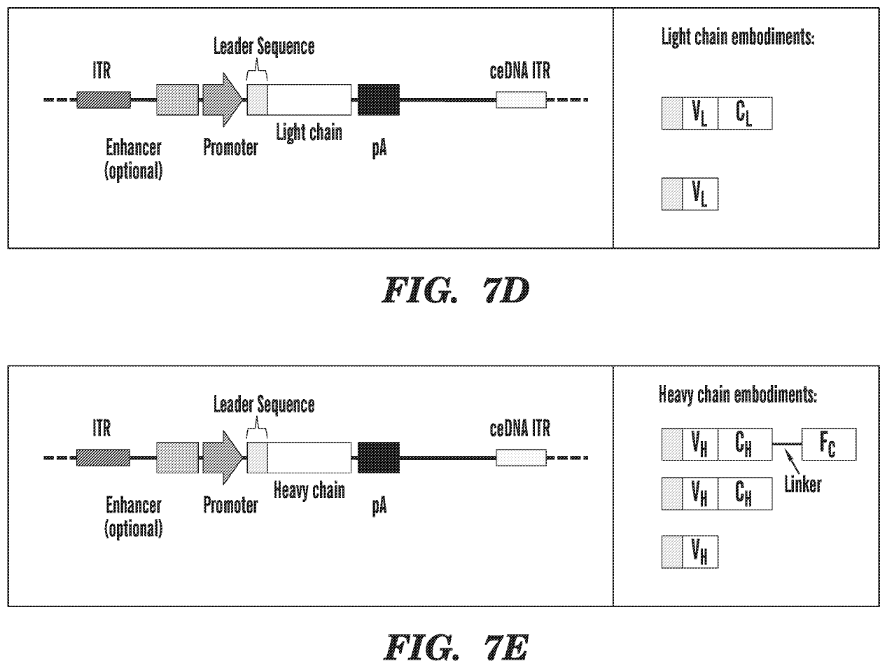

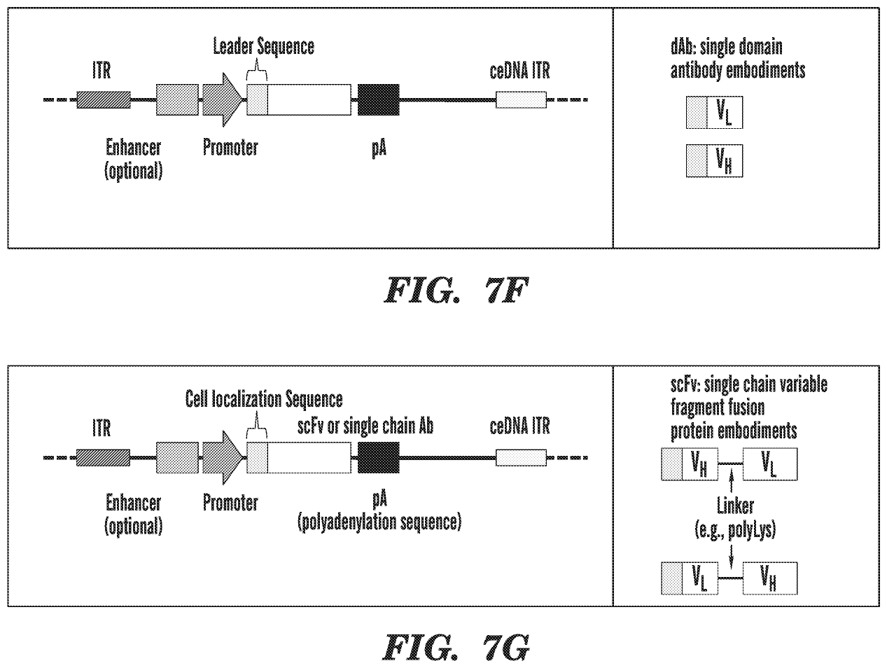

B. ceDNA vectors expressing an Antibody or Fusion Protein
A ceDNA Vector for Antibody or Fusion Protein Production Having One or More Sequences encoding a desired antibody can comprise regulatory sequences such as promoters, secretion signals, polyA regions, and enhancers. At a minimum, a ceDNA vector comprises one or more heterologous sequences encoding an antibody or fusion protein. Exemplary ceDNA vectors for antibody or fusion protein production are depicted in FIGS. 7A-7G.

In order to achieve highly efficient and accurate antibody or fusion protein assembly, it is specifically contemplated in some embodiments that the antibody, fusion protein, or individual antibody domains comprise an endoplasmic reticulum ER leader sequence to direct it to the ER, where protein folding occurs. For example, a sequence that directs the expressed protein(s) to the ER for folding.

In some embodiments, a cellular or extracellular localization signal (e.g., secretory signal, nuclear localization signal, mitochondrial localization signal etc.) is comprised in the ceDNA vector (see e.g., FIG. 10G) to direct the secretion or desired subcellular localization of the antibody or fusion protein such that the antibody or fusion protein can bind to intracellular target(s) (e.g., an intrabody) or extracellular target(s).

In certain embodiments, a ceDNA vector for antibody production can encode an intrabody, and in some embodiments, the intrabody can be a full length antibody as well as a single chain. Intrabodies can be used in a wide range of areas including treating viral disorders, and cellular disorders such as cancer, See e.g. U.S. Pat. No. 6,004,940.

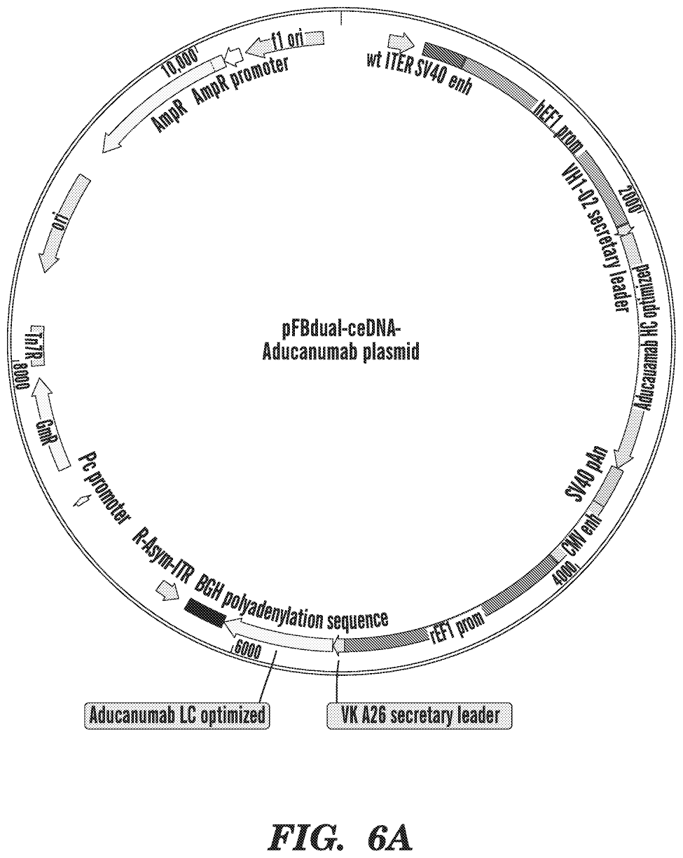

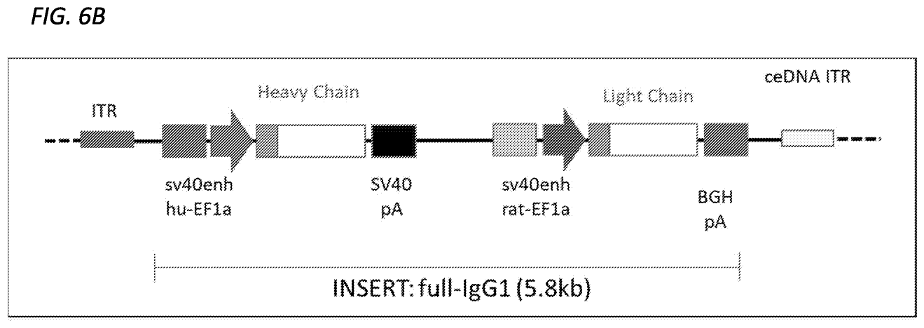

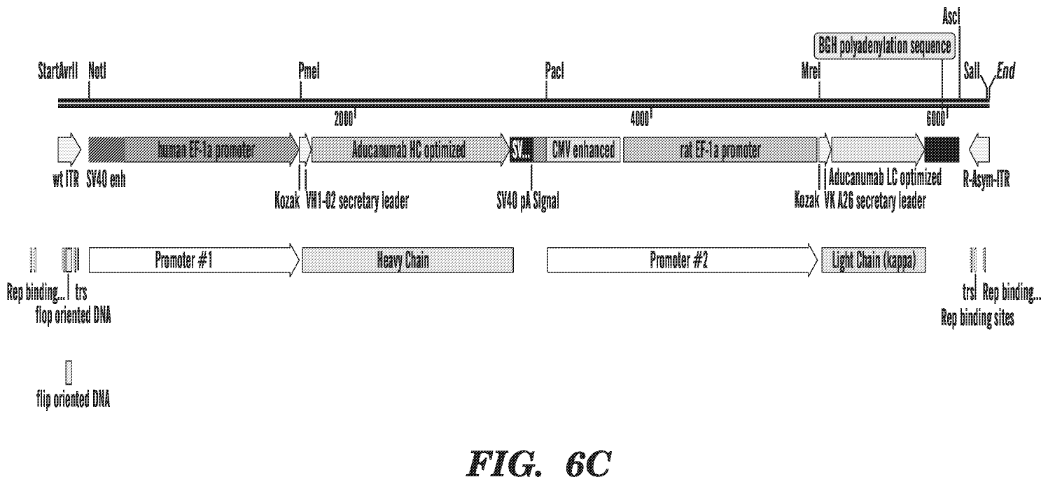

In some embodiments, a ceDNA vector for antibody or fusion protein production as described herein (e.g. see exemplary ceDNA vector shown in FIG. 6A) permits the assembly and expression of any desired antibody or fusion protein in a modular fashion. As used herein, the term “modular” refers to elements in a ceDNA expressing plasmid that can be readily removed from the construct. For example, modular elements in a ceDNA-generating plasmid comprise unique pairs of restriction sites flanking each element within the construct, enabling the exclusive manipulation of individual elements (see e.g., FIGS. 7A-7G). Thus, the ceDNA vector platform can permit the expression and assembly of any desired antibody or fusion protein configuration. Provided herein in various embodiments are ceDNA plasmid vectors that can reduce and/or minimize the amount of manipulation required to assemble a desired ceDNA vector encoding an antibody or fusion protein.

C. Exemplary Antibodies and Fusion Proteins Expressed by ceDNA Vectors

In particular, a ceDNA vector for antibody or fusion protein production as disclosed herein can encode, for example, but is not limited to, antibodies, antigen binding fragments, fusion proteins, as well as variants, and/or active fragments thereof, for use in the treatment, prophylaxis, and/or amelioration of one or more symptoms of a disease, dysfunction, injury, and/or disorder. In one aspect, the disease, dysfunction, trauma, injury and/or disorder is a human disease, dysfunction, trauma, injury, and/or disorder.

In some embodiments, a ceDNA vector for antibody or fusion protein production as disclosed herein can also encode co-factors or other polypeptides, sense or antisense oligonucleotides, or RNAs (coding or non-coding; e.g., siRNAs, shRNAs, micro-RNAs, and their antisense counterparts (e.g., antagoMiR)) that can be used in conjunction with the antibody or fusion protein expressed from the ceDNA. Additionally, expression cassettes comprising sequence encoding an antibody or fusion protein can also include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

In certain embodiments, multiple different antibodies and/or fusion proteins can be administered using one or more ceDNA vectors. Thus, it is specifically contemplated that one can express a desired number of antibodies and/or fusion proteins in a “cocktail” in a cell, tissue or subject.

The ceDNA vectors described herein can be used to deliver antibodies and fusion proteins for the treatment of e.g., cancer, autoimmune disease (e.g., rheumatoid arthritis, Crohn's disease), Alzheimer's disease, hypercholesterolemia, acute organ rejection, multiple sclerosis, post-menopausal osteoporosis, skin conditions (e.g., psoriasis, atopic dermatitis), asthma, or hemophilia.

ceDNA vectors as described herein can be used to express any desired therapeutic antibody or fusion protein. Exemplary therapeutic antibodies and fusion proteins include, but are not limited to, abciximab, Abaloparatide, Adalimumab, adalimumab-atto, ado-trastuzumab emtansine, aducanumab, alemtuzumab, alirocumab, atezolizumab, avelumab, bapineuzumab, basiliximab, belimumab, bevacizumab, bezlotoxumab, blinatumomab, blosozumab, Bococizumab, brentuximab vedotin, brodalumab, canakinumab, capromab pendetide, certolizumab pegol, cetuximab, concizumab, daclizumab, daratumumab, denosumab, dinutuximab, dupilumab, durvalumab, ecallantide, eculizumab, elotuzumab, emtansine, emicizumab, evolocumab, evinacumab, Factor IX-Fc antibody, Factor VIII-Fc antibody, golimumab, ibritumomab tiuxetan, idarucizumab, inclacumab, infliximab, infliximab-abda, infliximab-dyyb, ipilimumab, ixekizumab, lanadelumab, lodelcizumab, mepolizumab, natalizumab, necitumumab, nivolumab, obiltoxaximab, obinutuzumab, ocrelizumab, ofatumumab, olaratumab, omalizumab, orticumab, palivizumab, panitumumab, pembrolizumab, pertuzumab, pexelizumab, ralpancizumab, ramucirumab, ranibizumab, raxibacumab, reslizumab, rituximab, roledumab, romosozumab, secukinumab, siltuximab, solanezumab, sotatercept, tadocizumab, tocilizumab, trastuzumab, ustekinumab, vedolizumab, sarilumab, rituximab, guselkumab, inotuzumab ozogamicin, adalimumab-adbm, gemtuzumab ozogamicin, bevacizumab-awwb, benralizumab, emicizumab-kxwh, trastuzumab-dkst.

In one embodiment, the ceDNA vector comprises a nucleic acid sequence to express a therapeutic antibody or fusion protein that is functional for the treatment of disease. In a preferred embodiment, the therapeutic antibody or fusion protein does not cause an immune system reaction, unless so desired.

In one embodiment, an antibody is a therapeutic antibody or fusion protein expressed by the ceDNA vector that targets an immune checkpoint inhibitor (e.g., PDL1) and can be used for the treatment of e.g., cancer (e.g., solid tumors, breast cancer, lymphomas, liver cancer, ovarian cancer, lung cancer, colorectal cancer, leukemias, hematologic cancers, skin cancer, multiple myeloma etc.). In one embodiment, the therapeutic antibody or fusion protein targets a checkpoint inhibitor such as PDL1, CD47, mesothelin, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, α actinin4, TRP1/gp75, TRP2, gp100, Melan-A/MART1, gangliosides, WT1, EphA3, Epidermal growth factor receptor (EGFR), CD20, MART-2, MART-1, MUC1, MUC2, MUC16, MUM1, MUM2, MUMS, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGFIR, EPHA3, TRAILR1, TRAILR2, RANKL, fibroblast activating protease (FAP), TGF-beta, hyaluronic acid, collagen (e.g., collagen IV, tenascin C, or tenascin W), CD19, CD33, CD47, CD123, CD20, CD99, CD30, BCMA, CD38, CD22, SLAMF7, or NY-ESO1.

In one embodiment, the ceDNA vector expresses the evolocumab monoclonal antibody and is used for the treatment of hyperlipidemia. Evolocumab inhibits proprotein convertase subtilisin/kexin type 9 (PCSK9). PCSK9 is a protein that targets LDL receptors for degradation and thereby reduces the liver's ability to remove LDL-C, or “bad” cholesterol, from the blood. Evolocumab is further described in U.S. Pat. No. 8,999,341, which is incorporated herein by reference in its entirety.

Exemplary antibodies and fusion proteins expressed from a ceDNA vector for use in the methods and compositions as disclosed herein can be any antibody or fusion protein listed in Table 1, Table 2, Table 3A, Table 3B, Table 4 or Table 5 herein.

TABLE 1
FDA-Approved Antibodies and Fusion Proteins as Exemplary antibodies and fusion proteins.

| BLA | CHEMICAL | PROPRIETARY | REFERENCE(S), SEQ ID NO(S) IN |
|---|---|---|---|
| STN | (GENERIC) NAME | NAME | REFERENCE |
| 125118 | abatacept | ORENCIA ® | US20130330338A1, SEQ ID NO: 6 |
| | | | WO2017125402A1, SEQ ID NO: 2 |
| 103575 | abciximab | REOPRO ® | US20170002060A1, SEQ ID NO: 1, 2 |
| 125057 | adalimumab | HUMIRA ® | U.S. Pat. No. 8,921,526 |
| | | | US20180126000A1, SEQ ID NO: 66, 73 |
| | | | WO2017112536A1, SEQ ID NO: 23, 24 |
| 125387 | aflibercept | EYLEA ® | WO2014006113A1, SEQ ID NO: 1 |
| | | | WO2016073894A1, SEQ ID NO: 54 |
| 103948 | alemtuzumab | CAMPATH, | U.S. Pat. No. 8,592,149B2, |
| | | LEMTRADA ® | SEQ ID NO: 14 |
| | | | US20150112045A1, SEQ ID NO: 15, 16 |
| 125559 | alirocumab | PRALUENT ® | U.S. Pat. No. 8,062,640 |
| | | | US20180363000A1, SEQ ID NO: 11 |
| | | | US20170002060A1, SEQ ID NO: 160, 161 |
| 761034 | atezolizumab | TECENTRIQ ® | US20170239351A1, SEQ ID NO: 66, 67 |
| | | | U.S. Pat. No. 8,217,149B2, |
| | | | SEQ ID NO: 20, 21 |
| 761049 | avelumab | BAVENCIO ® | US20170239351A1, SEQ ID NO: 76, 77 |
| | | | US20140341917A1, SEQ ID NO: 24, 25 |
| 103764 | basiliximab | SIMULECT ® | US20150314007A1, SEQ ID NO: 20, 21 |
| | | | US20170002060A1, SEQ ID NO: 15, 16 |
| 125370 | belimumab | BENLYSTA ® | US20160281106A1, SEQ ID NO: 25, 26 |
| | | | US20170002060A1, SEQ ID NO: 17 |
| 761070 | benralizumab | FASENRA ® | US20170002060A1, SEQ ID NO: |
| | | | 183, 184, 185, 186 |
| | | | WO2008143878A1, SEQ ID NO: 1, 2, 3, 4 |
| 125085 | bevacizumab | AVASTIN ® | WO2018129533A1, SEQ ID NO: 65, 66 |
| | | | US20190016817A1, SEQ ID NO: 1, 2 |
| | | | WO2016073894A1, SEQ ID NO: 55, 56 |
| 761046 | bezlotoxumab | ZINPLAVA ® | WO2018111662A1, SEQ ID NO: 1, 5 |
| | | | U.S. Pat. No. 9,181,632B1, |
| | | | SEQ ID NO: 11, 12 |
| 125557 | blinatumomab | BLINCYTO ® | US20190038730A1, SEQ ID NO: 19 |
| | | | US20170362325A1, SEQ ID NO: 5 |
| | | | WO2016036678A1, SEQ ID NO: 25 |
| 125388 | brentuximab vedotin | ADCETRIS ® | US20180086734A1, SEQ ID NO: 10, 11 |
| | | | US20180036425A1, SEQ ID NO: 3, 4 |
| 761032 | brodalumab | SILIQ ® | US20170002060A1, SEQ ID NO: 238, 239 |
| | | | WO2006013107A1, SEQ ID NO: 427, 429 |
| 761068 | burosumab-twza | CRYSVITA ® | WO2015191312A1, SEQ ID NO: 7, 8, as |
| | | | KRN23 |
| 125319 | canakinumab | ILARIS ® | WO2018235056A1, SEQ ID NO: 17, 18 |
| | | | US20180186882A1, SEQ ID NO: 349, 350 |
| | | | US20130122008A1, SEQ ID NO: 1, 2 |
| 125160 | certolizumab pegol | CIMZIA ® | WO2017112536A1, SEQ ID NO: 25, 26 |
| | | | US20170002060A1, SEQ ID NO: 39, 40 |
| 125084 | cetuximab | ERBITUX ® | WO2018204717A1, SEQ ID NO: 124, 125 |
| | | | WO2017205465A2, SEQ ID NO: 1, 2 |
| | | | WO2017120536A1, SEQ ID NO: 7, 8 |
| 103749 | daclizumab | ZENAPAX ® | US20170226223A1, SEQ ID NO: 45, 46 |
| | | | US20170002060A1, SEQ ID NO: 41, 42 |
| | | | US20180256732A1, SEQ ID NO: 29, 30 |
| 761036 | daratumumab | DARZALEX ® | US20170044265A1, SEQ ID NO: 4, 5 |
| | | | WO2018002181A1, SEQ ID NO: 4, 5 |
| | | | WO2017162890A1, SEQ ID NO: 22, 29 |
| 125320 | denosumab | PROLIA ®, | U.S. Pat. No. 8,962,807, |
| | | XGEVA ® | U.S. Pat. No. 7,528,236, |
| | | | U.S. Pat. No. 7,364,736, |
| | | | U.S. Pat. No. 8,058,418, |
| | | | U.S. Pat. No. 8,409,578 |
| | | | US20170002060A1, SEQ ID NO: 49, 50 |
| 125516 | dinutuximab | UNITUXIN ® | US20140170155A1, SEQ ID NO: 3, 4 |
| 761055 | dupilumab | DUPIXENT ® | WO2018190990A1, SEQ ID NO: 345, 346 |
| | | | US20190040147A1, SEQ ID NO: 9, 10 |
| | | | US20170002060A1, SEQ ID NO: 359, 364 |
| 761069 | durvalumab | IMFINZI ® | US20190031766A1, SEQ ID NO: 643, 644 |
| | | | WO2018172286A1, SEQ ID NO: 13, 14 |
| | | | WO2018129533A1, SEQ ID NO: 25, 26 |
| 125166 | eculizumab | SOLIRIS ® | WO2018195034A1, SEQ ID NO: 10, 11 |
| | | | WO2018075758A1, SEQ ID NO: 5, 6 |
| | | | US20180311345A1, SEQ ID NO: 7, 8 |
| 761035 | elotuzumab | EMPLICITI ® | US20180222979A1, SEQ ID NO: 36, 37 |
| | | | US20170002060A1, SEQ ID NO: 289, 290 |
| | | | US20180185348A1, SEQ ID NO: 1, 2 |
| 761083 | emicizumab-kxwh | HEMLIBRA ® | US20180011114A1, SEQ ID NO: 9, 10, 11, 12, |
| | | | as ACE910 |
| 761077 | erenumab-aooe | AIMOVIG ® | U.S. Pat. No. 9,102,731B2 |
| 103795 | etanercept | ENBREL ® | US20180291084A1, SEQ ID NO: 10 |
| | | | WO2016009049A1, SEQ ID NO: 4 |
| | | | US20160160279A1, SEQ ID NO: 1 |
| 125522 | evolocumab | REPATHA ® | U.S. Pat. No. 8,030,457 |
| | | | US20170002060A1, SEQ ID NO: 158, 159 |
| 761060 | gemtuzumab | MYLOTARG ® | WO2018129533A1, SEQ ID NO: 67, 68 |
| | ozogamicin | | US20190002560A1, SEQ ID NO: 248, 249 |
| | | | US20180148514A1, SEQ ID NO: 243, 244 |
| 125289 | golimumab | SIMPONI ® | WO2018147915A1, SEQ ID NO: 36, 37 |
| | | | U.S. Pat. No. 7,250,165B2 cited by |
| | | | US20170002060A1 |
| 125433 | golimumab | SIMPONI ARIA ® | US20170002060A1, SEQ ID NO: 63, 64 |
| | | | U.S. Pat. No. 7,250,165B2, SEQ ID NO: 7, 8 |
| | | | WO2013087912A1, SEQ ID NO: 6, 7 |
| | | | All - VH/VL |
| 761061 | guselkumab | TREMFYA ® | WO2018093841A1, SEQ ID NO: 106, 116 |
| | | | U.S. Pat. No. 9,803,010B2, |
| | | | SEQ ID NO: 44, 45 |
| 761065 | ibalizumab-uiyk | TROGARZO ® | U.S. Pat. No. 8,637,024B2, |
| | | | SEQ ID NO: 15, 19, VH/VL |
| 125019 | ibritumomab tiuxetan | ZEVALIN ® | WO2018129533A1, SEQ ID NO: 59, 60 |
| | | | US20170002060A1, SEQ ID NO: 71, 72 |
| | | | U.S. Pat. No. 9,683,985B2, |
| | | | SEQ ID NO: 41, 42 |
| 761025 | idarucizumab | PRAXBIND ® | U.S. Pat. No. 8,486,398 |
| 103772 | infliximab | REMICADE ® | US20160153020A1, SEQ ID NO: 41, 42 |
| | | | US20180256732A1, SEQ ID NO: 22, 23 |
| 761040 | inotuzumab | BESPONSA ® | WO2018193231A1, SEQ ID NO: 25, 26 |
| | ozogamicin | | US20170002060A1, SEQ ID NO: 299, 304 |
| | | | U.S. Pat. No. 8,153,768B2, |
| | | | SEQ ID NO: 28, 30 |
| 125377 | ipilimumab | YERVOY ® | US20150283234A1 |
| | | | WO2018204343A1, SEQ ID NO: 84, 85 |
| | | | WO2018049042A1, SEQ ID NO: 1, 2 |
| 125521 | ixekizumab | TALTZ ® | WO2019027828A1, SEQ ID NO: 4, 5 |
| | | | U.S. Pat. No. 7,838,638 |
| | | | U.S. Pat. No. 8,110,191 |
| 125526 | mepolizumab | NUCALA ® | WO2009068649A2, SEQ ID NO: 65, 66 |
| | | | WO2009120927A2, SEQ ID NO: 19, 21 |
| | | | Ref. by US20170002060A1 |
| 125104 | natalizumab | TYSABRI ® | U.S. Pat. No. 5,840,299A, |
| | | | SEQ ID NO: 2, 4 VL/VH ref by |
| | | | US20170002060A1 |
| | | | WO2010121141A1, SEQ ID NO: 1, 2 |
| 125547 | necitumumab | PORTRAZZA ® | US20170002060A1, SEQ ID NO: 320, 324 |
| 125554 | nivolumab | OPDIVO ® | US20190031766A1, SEQ ID NO: 535, 536 |
| | | | WO2018183408A1, SEQ ID NO: 23, 24 |
| | | | WO2018150326A1, SEQ ID NO: 98, 99 |
| 125509 | obiltoxaximab | ANTHIM ® | WO2017186928A1, SEQ ID NO: 616, 617 |
| 125486 | obinutuzumab | GAZYVA ® | US20190008869A1, SEQ ID NO: 3, 4 |
| | | | US20190022092A1, SEQ ID NO: 367, 368 |
| 761053 | ocrelizumab | OCREVUS ® | U.S. Pat. No. 8,679,767B2, |
| | | | SEQ ID NO: 11, 19 |
| | | | US20180327505A1, SEQ ID NO: 13, 14 |
| 125326 | ofatumumab | ARZERRA ® | US20180169189A1, SEQ ID NO: 23, 24 |
| | | | VH/VL |
| | | | WO2009086072A2, SEQ ID NO: 1, 2 VH/VL |
| 761038 | olaratumab | LARTRUVO ® | WO2018022407A1, SEQ ID NO: 26, 28 |
| | | | US20170137523A1, SEQ ID NO: 11, 12 |
| 103976 | omalizumab | XOLAIR ® | US20170002060A1, SEQ ID NO: 110, 111 |
| | | | VH/VL |
| | | | US20150314007A1, SEQ ID NO: 22, 23 |
| | | | US20150140608A1, SEQ ID NO: 1, 2 |
| 103770 | palivizumab | SYNAGIS ® | WO2018013483A1, SEQ ID NO: 1, 2 VH/VL |
| | | | U.S. Pat. No. 7,786,273B2, |
| | | | SEQ ID NO: NO: 1, 2 VH/VL |
| 125147 | panitumumab | VECTIBIX ® | WO2018156802A1, SEQ ID NO: 1, 4 |
| | | | WO2017060322A2, SEQ ID NO: 237, 238 |
| 125514 | pembrolizumab | KEYTRUDA ® | WO2018183408A1, SEQ ID NO: 21, 22 |
| | | | WO2018150326A1, SEQ ID NO: 50, 51 |
| | | | WO2018106588A1, SEQ ID NO: 32, 33 |
| 125409 | pertuzumab | PERJETA ® | WO2018160654A2, SEQ ID NO: 11, 12 |
| | | | WO2018140831A2, SEQ ID NO: 11, 16 |
| | | | US20120107391A1, SEQ ID NO: 16, 17 |
| 125477 | ramucirumab | CYRAMZA ® | WO2018093668A1, SEQ ID NO: 1, 2 VH/VL |
| | | | WO2018081512A1, SEQ ID NO: 1, 2 VH/VL |
| 125156 | ranibizumab | LUCENTIS ® | WO2018211529A1, SEQ ID NO: 3, 4 |
| | | | WO2017117464A1, SEQ ID NO: 5, 6 |
| 761033 | reslizumab | CINQAIR ® | US20170002060A1, SEQ ID NO: 181, 182 |
| | | | WO1995035375A1, ref. by |
| | | | US20170002060A1 |
| 103705 | rituximab | RITUXAN ® | WO2018129533A1, SEQ ID NO: 55, 56 |
| | | | WO2018033482A1, SEQ ID NO: 4, 5 |
| | | | US20190008869A1, SEQ ID NO: 1, 2 |
| 761037 | sarilumab | KEVZARA ® | US20170166646A1, SEQ ID NO: 2, 3 |
| | | | US20170252434A1, SEQ ID NO: 1, 2 |
| | | | US20170002060A1, SEQ ID NO: 207, 208 |
| 125504 | secukinumab | COSENTYX ® | WO2006013107A1 |
| | | | US20170002060A1, SEQ ID NO: 225, 226 |
| | | | US20170304439A1, SEQ ID NO: 8, 10, VH/VL |
| 125496 | siltuximab | SYLVANT ® | US20170002060A1, SEQ ID NO: 197, 198 |
| | | | US20180236032A1, SEQ ID NO: 4, 5 |
| 761067 | tildrakizumab-asmn | ILUMYA ® | US20170002060A1, SEQ ID NO: 245, 246 |
| | | | U.S. Pat. No. 8,263,748B2, SEQ ID NO: 7, 17 |
| 125276 | tocilizumab | ACTEMRA ® | WO2017202387A1, SEQ ID NO: 1, 2 |
| | | | US20170074890A1, SEQ ID NO: 1, 2 |
| | | | US20170152319A1, SEQ ID NO: 1, 2 |
| 103792 | trastuzumab | HERCEPTIN ® | WO2018160654A2, SEQ ID NO: 13, 14 |
| | | | WO2018129533A1, SEQ ID NO: 57, 58 |
| 761044 | ustekinumab | STELARA ® | WO2018024770A1, SEQ ID NO: 1, 2 |
| | | | US20190025325A1, SEQ ID NO: 1, 2 |
| | | | WO2017112536A1, SEQ ID NO: 19, 20 |
| 125476 | vedolizumab | ENTYVIO ® | US20170002060A1, SEQ ID NO: 154, 155 |
| | | | US20120282249A1, SEQ ID NO: 2, 4 |
| | | | WO2008115504A2, SEQ ID NO: 11, 12 |

TABLE 2
Exemplary antibodies and fusion proteins for expression by ceDNA
vectors useful in the methods and compositions described herein.

| Target | Exemplary Antibody | Reference Information |
|---|---|---|
| Transferrin Receptor | | 2015/0110791 A1 |
| Alpha4-integrin | Natalizumab, TYSABRI ® | 5,840,299, 6,033,665, 6,602,503, |
| | | 5,168,062, 5,385,839, 5,730,978 |
| EGFR | Cetuximab, ERBITUX ®, | U.S. Pat. No. 6,217,866B1 |
| | panitumumab, necitumumab, | |
| | PORTRAZZA ® | |
| CD25 (alpha chain of IL- | Basiliximab, SIMULECT ®, | U.S. Pat. No. 6,521,230B1 |
| 2 receptor) | daclizumab, ZINBRYTA ® | |
| TNFalpha | REMICADE ®, SIMPONI ®, | US20080033149A1, |
| | adalimumab, HUMIRA ®, | US20120258114A1, |
| | certolizumab pegol, CIMZIA ®, | US20030157061 |
| | golimumab, infliximab | |
| CTLA4 | Orencia, ipilimumab, | WO1993000431A1, |
| | YERVOY ® | U.S. Pat. No. 8,784,815B2 |
| IgE | XOLAIR ®, omalizumab, | WO1993004173A1 |
| | ligelizumab | |
| C5 | Eculizumab, SOLIRIS ®, | WO1995029697A1 |
| | REGN3918, pexelizumab | |
| IL-6R | Sarilumab, satralizumab, | U.S. Pat. No. 8,017,121B2 |
| | ACTEMRA ®, tocilizumab, | |
| | atlizumab | |
| BAFF (BLyS) | Belimumab, BENLYSTA ® | WO1998018921A1, |
| | | WO2003055979A2 |
| VEGF-A | Ranibizumab, LUCENTIS ®, | WO1998045331A2, |
| | AVASTIN ®, brolucizumab, | WO1994010202A1, US20140086829, |
| | bevacizumab, ramucirumab, | US2013067098 Lien and Lowman, |
| | CYRAMZA ® | In: Chemajovsky, 2008, Therapeutic |
| | | Antibodies. Handbook of |
| | | Experimental Pharmacology 181, |
| | | Springer-Verlag, Berlin Heidelberg |
| | | 131-150 |
| P-selectin | Inclacumab, LC1004-002, | U.S. Pat. No. 7,563,441; |
| | R04905417 | U.S. Pat. No. 8,039,596; |
| | | U.S. Pat. No. 7,432,359 |
| HER2 | Trastuzumab, HERCEPTIN ®, | U.S. Pat. No. 7,879,325 |
| | pertuzumab, PERJETA ®, | |
| | KADCYLA ® | |
| PCSK9 | Alirocumab, bococizumab, | U.S. Pat. No. 8,030,457, |
| | evolocumab, REPATHA ®, | U.S. Pat. No. 8,710,192 |
| | lodelcizumab, ralpancizumab | |
| PD-L1 | Atezolizumab, TENCENTRIQ ®, | U.S. Pat. No. 8,217,149 |
| | avelumab, BAVENCIO ®, | |
| | durvalumab, IMFINZI ® | |
| oxLDL | Orticumab, BI-204, MLDL- | U.S. Pat. No. 8,318,161 |
| | 1278A, R7418 | |
| Dabigatran | Idarucizumab, PRAXBIND ® | U.S. Pat. No. 8,486,398 |
| Parathyroid hormone- | Abaloparatide, TYMLOS ® | U.S. Pat. No. 8,747,382, |
| related protein (PTHrP) | | U.S. Pat. No. 7,803,770; |
| | | U.S. Pat. No. 8,148,333 |
| RANKL | Denosumab, PROLIA ®, | U.S. Pat. No. 8,962,807; |
| | XGEVA ® | U.S. Pat. No. 7,528,236; |
| | | U.S. Pat. No. 7,364,736; |
| | | U.S. Pat. No. 8,058,418; |
| | | U.S. Pat. No. 8,409,578 |
| NGF | ranezumab, fasinumab | US20040237124 |
| PD1-receptor | Pembrolizumab, lambrolizumab, | US2012135408 |
| | KEYTRUDA ® | |
| Alpha4beta7 integrin | Vedolizumab, ENTYVIO ® | US2012151248 |
| Integrun alpha4beta7 | Vedolizumab, ENTYVIO ® | US2012151248 |
| IL17a | TALTZ ®, ixekizumab, | US20130202610 |
| | secukinumab, COSENTYX ® | |
| CSa | BNJ378, BNJ383 | US20130224187 |
| PD1 | Cemiplimab, nivolumab, | US2013173223 |
| | OPDIVO ® | |
| PDGFRbeta | 3299N, olaratumab, | US20140193402 |
| | LARTRUVO ® | |
| TiKA | BXhVH5VL1 | US20150183885 |
| TrkA | GBR, VH5(P60A, T62 S)VL1 | US20150183885 |
| Beta-amyloid | crenezumab, MEDI1814, | US20150246963 |
| | gantenerumab, aducanumab, | |
| | BAN2401, solenezumab, | |
| | LY3002813, bapinezumab | |
| activin | REGN 2477, | US20170260275, WO2015162590 |
| | BIMAGRUMAB ®, garetosmab | |
| CD33 | Gemtuzumab, MYLOTARG ® | U.S. Pat. No. 5,585,089 |
| CD19 | Blinatumomab, BLINCYTO ®, | U.S. Pat. No. 7,235,641; |
| | inebilizumab | U.S. Pat. No. 7,575,923; |
| | | U.S. Pat. No. 7,635,472; |
| | | U.S. Pat. No. 8,247,194 |
| IL-6 | Siltuxumab, SYLVANT ® | U.S. Pat. No. 7,612,182 |
| NGF | Fulraintin, 4D4, AMG-403, | U.S. Pat. No. 7,601,818 |
| | lN.142160443 | |
| | Fulranumab; 4D4, AMG- 403, | U.S. Pat. No. 7,988,967 |
| | JNJ- | U.S. Pat. No. 8,552,157 |
| | | U.S. Pat. No. 8,048,421 |
| CD20 | Ocrelizumab, OCREVUS ®, | U.S. Pat. No. 8,337,847, |
| | ofatumumab, ARZERRA ®, | WO1994011026A3 |
| | rituximab, RITUXAN ®, | |
| | obinutuzumab, ibritumomab, | |
| GFRa3 | H4H2364S | U.S. Pat. No. 8,968,736 |
| CORP | 01, cluster headache | U.S. Pat. No. 9,115,194 |
| CGRP | galcanezumab, erenumab | U.S. Pat. No. 9,115,194, US20120294802, |
| | | U.S. Pat. No. 9,102,731 |
| TrkA | BXhVHl ® | W02009098238 |
| TrkA | HUVHWOV ® | W02009098238 |
| Annexin IV or a | | W02014116880 |
| phospholipid; and (b) a | | |
| complement inhibitor | | |
| Nav 1.7 | H1Hl015B | W02014159595 |
| Factor D | Fab238, lampalizumab | WO2009134711, U.S. Pat. No. 8,273,352 |
| Rhesus factor antigen | Roledumab, DMATRIA ® | WO2010100383 |
| TGFbeta | DOM23h-271-12 | WO2011012609 |
| Alpha-synuclein | BIIB054 | WO2018178950A1, SEQ ID NO: 8, 9 |
| | | VH/VL |
| | RG7935 | US20160324852A1 |
| Bacillus anthracis | raxibacumab | US20130028920A1 |
| protective antigen | | |
| | obiltoxaximab, ANTHIM ® | WO2017186928A1, SEQ ID |
| | | NO: 616, 617 |
| ANGPTL-3 | evinacumab | US20170312359A1, SEQ ID NO: 1, 5 |
| | | VH/VL |
| | | WO2017151783A1, SEQ ID |
| | | NO: 10, 11 |
| B7RP1 | prezalumab | WO2017186928A1 |
| BAFF | VAY736 | U.S. Pat. No. 8,106,163B2, |
| | | SEQ ID NO: 71, 75 |
| BDCA2 | BIIB059 | WO2017189827A1, SEQ ID |
| | | NO: 9, 10 |
| | | US20180362652A1, SEQ ID NO: 3, 4 |
| C1 | BIVV009 | WO2018170145A1, SEQ ID |
| | | NO: 22, 23 |
| CD22 | inotuzumab | WO2018193231A1, SEQ ID |
| | | NO: 25, 26; WO2018193231A1, |
| | | SEQ ID NO: 25, 26; |
| | | U.S. Pat. No. 8,153,768B2, |
| | | SEQ ID NO: 28, 30 |
| CD30 | Brentuximab vedotin, | US20180086734A1, SEQ ID |
| | ADCETRIS ® | NO: 10, 11; US20180036425A1, SEQ |
| | | ID NO: 3, 4 |
| CD38 | Daratumumab, DARZALEX ® | US20170044265A1, SEQ ID |
| | | NO: 4, 5; W02018002181A1, SEQ ID |
| | | NO: 4, 5; WO2017162890A1, SEQ ID |
| | | NO: 22, 29 |
| CD52 | Alemtuzumab, LEMTRADA ® | U.S. Pat. No. 8,592,149B2, |
| | | SEQ ID NO: 14; US20150112045A1, |
| | | SEQ ID NO: 15, 16 |
| CD319 (SLAMF7) | Elotuzumab, EMPLICITI ® | US20180222979A1, SEQ ID |
| | | NO: 36, 37; US20170002060A1, SEQ |
| | | ID NO: 289, 290; US20180185348A1, |
| | | SEQ ID NO: 1, 2 |
| Clostridium toxins A and | Bezlotoxumab, ZINPLAVA ® | WO2018111662A1, SEQ ID NO: 1, 5; |
| B | | U.S. Pat. No. 9,181,632B1, |
| | | SEQ ID NO: 11, 12 |
| Fel d1 | REGN1908 | WO2018118713A1, SEQ ID |
| | | NO: 18, 26 VH/VL |
| | REGN1909 | WO2018118713A1, SEQ ID |
| | | NO: 306, 314 VH/VL |
| GDF8 | Trevogrumab | U.S. Pat. No. 8,840,894B2 |
| | | WO2016168613A1, SEQ ID |
| | | NO: 360, 368 VH/VL |
| Glycolipid GD2 | Dinutuximab, UNITUXIN ®, | US20140170155A1, SEQ ID NO: 3, 4 |
| | Dinutuximab beta, ISQUETTE ® | |
| Glycoprotein IIb/IIa | Abciximab, REOPRO ® | US20170002060A1, SEQ ID NO: 1, 2 |
| receptor | | |
| GM-CSF | Namilumab | WO2017076804A1, SEQ ID |
| | | NO: 11, 12 |
| | mavrilimumab | U.S. Pat. No. 8,263,075B2 |
| | | U.S. Pat. No. 8,506,960B2 |
| | | WO2017202879A1, SEQ ID NO: 2, 3 |
| IFNalpha receptor | anifrolumab | US20110059078A1 |
| | | US20160251441A1, SEQ ID NO: 1, 2 |
| | | VH/VL as MEDI-546 |
| IG2kappa | SHP647 | |
| IL-1beta | Canakinumab, ILARIS ® | WO2018235056A1, SEQ ID |
| | | NO: 17, 18; US20180186882A1, SEQ |
| | | ID NO: 349, 350; US20130122008A1, |
| | | SEQ ID NO: 1, 2 |
| IL-3R | Dupilumab | WO2018190990A1, SEQ ID |
| | | NO: 345, 346; US20190040147A1, |
| | | SEQ ID NO: 9, 10; |
| | | US20170002060A1, SEQ ID |
| | | NO: 359, 364 |
| IL-5 | FENSENRA ®, mepolizumab, | WO2009068649A2, SEQ ID |
| | GS3511294, reslizumab, | NO: 65, 66; WO2009120927A2, SEQ |
| | CINQUIR ® | ID NO: 19, 21; Ref. by |
| | | US20170002060A1 |
| IL-5R (CD125) | Benralizumab, FASRENRA ® | US20170002060A1, SEQ ID NO: |
| | | 183, 184, 185, 186 |
| | | WO2008143878A1, SEQ ID |
| | | NO: 1, 2, 3, 4 |
| IL-17 receptor | Brodalumab, SILIQ ® | US20170002060A1, SEQ ID |
| | | NO: 238, 239 |
| | | WO2006013107A1, SEQ ID |
| | | NO: 427, 429 |
| IL-23 | TREMFYA ®, guselkumab | WO2018093841A1, SEQ ID |
| | | NO: 106, 116 |
| | | U.S. Pat. No. 9,803,010B2, |
| | | SEQ ID NO: 44, 45 |
| | mirikizumab | WO2019012531A1 |
| IL-31 | nemolizumab | US20180346543A1 |
| IL-33 | REGN3500 | WO2018102597A1, SEQ ID |
| | | NO: 274, 282 VH/VL |
| | MEDI3506 | |
| | GSK3772847 (formally CNTO | |
| | 716) | |
| Influenza A | MEDI8852 | |
| Integrin alphaiibbeta3 | tadocizumab | WO2016011264A1 |
| kallikrein | SHP643 | EP3390439A1 |
| | ecallantide, KALBITOR ® | US20160252527A1, SEQ ID NO: 3 |
| | | US20070213275A1, SEQ ID NO: 1 |
| | lanadelumab | US20180306807A1, SEQ ID |
| | | NO: 81, 82 VH/VL |
| | DX-2930 | US20180118851A1, SEQ ID |
| | | NO: 9, 10 |
| | | US20150362492A1, SEQ ID |
| | | NO: 9, 10 VH/VL |
| LAG-3 | REGN3767 | WO2018222722A2 |
| LEPR | | |
| LINGO-1 | Opininumab | |
| Middle eastern repiratory | REGN3048, REGN3049, | US20190030187A1 |
| coronavirus | REGN3050, REGN3051 | |
| Myostatin | Domagrozumab, RG6206 | |
| NGF | 42160443 | |
| PSMA | Capromab pendetide, | WO2006076525A2 |
| | PROSTASCINT ® | |
| RSV F protein | Palivizumab, SYNAGIS ® | WO2018013483A1, SEQ ID NO: 1, 2 |
| | | VH/VL |
| | | U.S. Pat. No. 7,786,273B2, |
| | | SEQ ID NO: NO: 1, 2 VH/VL |
| | MEDI8897 | WO2018160722A1, SEQ ID NO: 1, 2 |
| | SP0232 | |
| Sclerostin | romosozumab | WO2017153541A1, SEQ ID |
| | | NO: 11, 12 |
| SOST | blosozumab | W02018115880A1, SEQ ID |
| | | NO: 70, 81 VH/VL possibly |
| Staph aureus toxin | suvratoxumab | WO2017075188A2, SEQ ID |
| | | NO: 9, 10 as MEDI4893 |
| Tau | BIIB092 | WO2018231254A1, SEQ ID |
| | | NO: 14, 15 |
| | RG6100 | WO2019025595A1 |
| TFPI | concizumab | WO2017186928A1, SEQ ID |
| | | NO: 176, 177 |
| | NN7415 | |
| Thymic stromal | tezepelumab | WO2018191479A1, SEQ ID |
| lymphoprotein | | NO: 105, 106 |

Bispecific antibody targets

| | | |
|---|---|---|
| Alpha3beta7/alphaEbeta7 | etrolizumab | US20180086833A1 |
| | | WO2017019673A2, SEQ ID |
| | | NO: 31, 32 VH/VL |
| CD20/CD3 | REGN1979 | |
| Factor IX/factor X | Emicizumab, HEMLIBRA ® | US20180011114A1, SEQ ID |
| | | NO: 9, 10, 11, 12, as ACE910 |
| IL-4/IL-13 | SAR156597 | US20170145089A1, SEQ ID |
| | | NO: 1, 2, 3, 4 VH/VL |
| IL-12/IL-23 | Ustekinumab, STELARA ® | WO2018024770A1, SEQ ID NO: 1, 2 |
| | | US20190025325A1, SEQ ID NO: 1, 2 |
| | | WO2017112536A1, SEQ ID |
| | | NO: 19, 20 |
| IL-33/STR2 | AMG 282, RG6149 | |
| Psl/PcrV | MEDI3902 | |
| VEGF-A/Ang2 | RG7716 | WO2017218977A2, SEQ ID |
| | | NO: 13, 14, 15, 16 |

Antibody-like proteins

| | | |
|---|---|---|
| Activin receptor type 2A- | ACE-011, SOTATERCEPT ® | US20180161426A1, SEQ ID NO: 7 |
| IgG-Fc fusion protein | | US20170304397A1, SEQ ID NO: 7 |
| (functions as a TGF-β | | |
| ligand trap) | | |

TABLE 3A
Exemplary antibodies to be expressed by ceDNA vectors include, but are not limited
to antibody therapeutics approved in the European Union or United States.

| International Non- | BRAND | | Indication first |
|---|---|---|---|
| Proprietary Name | NAME | Target; Format | approved |
| Brodalumab | SILIQ ®, | IL-17RA; Human IgG2 | Plaque psoriasis |
| | LUMICEF ®, | | |
| | KYNTHEUM ® | | |
| Avelumab | BAVENCIO ® | PD-L1; Human IgG1 | Merkel cell carcinoma |
| Dupilumab | DUPIXENT ® | IL-4Rα; Human IgG4 | Atopic dermatitis |
| Ocrelizumab | OCREVUS ® | CD20; Humanized IgG1 | Multiple sclerosis |
| Durvalumab | IMFINZI ® | PD-L1; Human IgG1 | Bladder cancer |
| Sarilumab | KEVZARA ® | IL-6R; Human IgG1 | Rheumatoid arthritis |
| Guselkumab | TREMFYA ® | IL-23 p19; Human IgG1 | Plaque psoriasis |
| Inotuzumabozogamicin | BESPONSA ® | CD22; Humanized IgG4; | Acute lymphoblastic |
| | | ADC | leukemia |
| Benralizumab | FASENRA ® | IL-5R α; Humanized IgG1 | Asthma |
| Emicizumab | HEMLIBRA ® | Factor IXa, X; Humanized | Hemophilia A |
| | | IgG4, bispecific | |

TABLE 3B
Exemplary antibodies to be expressed by ceDNA vectors include, but are not limited to
antibody therapeutics in regulatory review in the European Union or United States

| International | | | |
|---|---|---|---|
| Non-Proprietary | Brand name | | |
| Name | proposed | Target; Format | Indication under reviewed |
| Ibalizumab | (Pending) | CD4; Humanized IgG4 | HIV infection |
| Burosumab | (Pending) | FGF23; Human IgG1 | X-linked hypophosphatemia |
| Tildrakizumab | (Pending) | IL-23 p19; Humanized | Plaque psoriasis |
| | | IgG1 | |
| Caplacizumab | (Pending) | von Willebrand factor; | Acquired thrombotic |
| | | Humanized Nanobody | thrombocytopenic purpura |
| Erenumab | AIMOVIG ® | CGRP receptor; | Migraine prevention |
| | | Human IgG2 | |
| Fremanezumab | (Pending) | CGRP; Humanized | Migraine prevention |
| | | IgG2 | |
| Galcanezumab | (Pending) | CGRP; Humanized | Migraine prevention |
| | | IgG4 | |
| Romosozumab | EVENITY ® | Sclerostin; Humanized | Osteoporosis in postmenopausal |
| | | IgG2 | women at increased risk of |
| | | | fracture |
| Mogamulizumab | POTELIGEO ® | CCR4; Humanized | Cutaneous T cell lymphoma |
| | | IgG1 | |

Table 4: Exemplary antibodies to be expressed by ceDNA vectors include, but are not limited to Antibody therapeutics for non-cancer indications in late-stage clinical studies. Companies commercially developing or clinically testing the antibodies are as follows: 1. Novartis, 2. LFB Group, 3. Shire, 4. Prothena Therapeutics Ltd., 5. Omeros Corporation, 6. Alexion Pharmaceuticals Inc., 7. AstraZeneca/MedImmune LLC, 8. Boehringer Ingelheim Pharmaceuticals, AbbVie, 9. Genentech, 10. Shire, 11. R-Pharm, 12. Chugai Pharmaceuticals/Roche, 13. NovImmune SA, 14. CytoDyn, 15. Biogen, 16. Hoffmann-La Roche, 17. Alder Biopharmaceuticals, 18. Regeneron Pharmaceuticals, 19. Pfizer; Eli Lilly & Company, 20. Horizon Pharma USA

TABLE 4

| INN or code name | Molecular format | Target | Late-stage study indication(s) |
|---|---|---|---|
| Crizanlizumab¹ | Humanized IgG2 | CD62 (aka P- | Sickle cell disease |
| | | selectin) | |
| Roledumab² | Human IgG1 | RhD | Rhesus disease |
| Lanadelumab³ | Human IgG1 | Plasma kallikrein | Hereditary angioedema attacks |
| NEOD001⁴ | Humanized IgG1 | Amyloid | Primary systemic |
| | | | amyloidosis |
| OMS721⁵ | Human mAb | MASP-2 | Atypical hemolytic uremic |
| | | | syndrome |
| Ravulizumab | Humanized mAb | C5 | Paroxysmal nocturnal |
| (ALXN1210)⁶ | | | hemoglobinuria, atypical |
| | | | hemolytic uremic syndrome |
| Tezepelumab⁷ | Human IgG2 | Thymic stromal | Severe uncontrolled asthma |
| | | lympho-poietin | |
| Tralokinumab⁷ | Human IgG4 | IL-13 | Atopic dermatitis |
| Risankizumab⁸ | Humanized IgG1 | IL-23 p19 | Psoriasis; Crohn's disease |
| Anifrolumab⁷ | Human IgG1 | IFN α, β, ω receptor | Systemic lupus erythematosus |
| | | 1 | |
| Etrolizumab⁹ | Humanized IgG1 | α4-β7/αE-β7 integrin | UC; Crohn's disease |
| | | receptor | |
| SHP-647¹⁰ | Human IgG2 | Mucosal addressin | UC; Crohn's disease |
| | | cell adhesion | |
| | | molecule | |
| Olokizumab¹¹ | Humanized IgG4 | IL-6 | Rheumatoid arthritis |
| Inebilizumab⁷ | Humanized IgG1 | CD19 | Neuromyelitis optica and |
| | | | neuromyelitis optica spectrum |
| | | | disorders |
| Satralizumab¹² | Humanized IgG2 | IL-6R | Neuromyelitis optica and |
| | | | neuromyelitis optica spectrum |
| | | | disorders |
| Emapalumab¹³ | Human IgG1 | IFN γ | Primary hemophagocytic |
| | | | lymphohistiocytosis |
| PRO-140, PA14¹⁴ | Humanized IgG4 | CCR5 | HIV infection |
| Aducanumab¹⁵ | Human IgG1 | Amyloid β | Alzheimer's disease |
| Crenezumab⁹ | Humanized IgG4 | Amyloid β | Alzheimer's disease |
| Gantenerumab¹⁶ | Human IgG1 | Amyloid β | Alzheimer's disease |
| Eptinezumab¹⁷ | Humanized IgG1 | CGRP | Migraine prevention, chronic |
| | | | migraine |
| Fasinumab¹⁸ | Human IgG4 | NGF | Pain due to osteoarthritis |
| | | | of knee or hip, chronic low |
| | | | back pain |
| Tanezumab¹⁹ | Humanized IgG2 | NGF | Pain due to osteoarthritis |
| | | | of knee or hip, chronic low |
| | | | back pain, cancer pain due to |
| | | | bone metastasis |
| Teprotumumab²⁰ | Human IgG1 | IGF-1R | Thyroid eye disease |
| Lampalizumab⁹ | Humanized IgG1 | Complement Factor | Geographic atrophy associated |
| | Fab | D | with dry age-related macular |
| | | | degeneration |
| Brolucizumab¹ | Humanized scFv | VEGF-A | Neovascular age- |
| | | | related macular |
| | | | degeneration |

Table 5: Exemplary antibodies to be expressed by ceDNA vectors include, but are not limited to antibody therapeutics for cancer indications in late-stage clinical studies. Companies commercially developing or clinically testing the antibodies in Table 4 are as follows: 1. Actinium Pharmaceuticals, 2. Sanofi, 3. TG Therapeutics, 5. MorphoSys, 6. Pfizer, 8. Viventia Bio, 10. Jiangsu HengRui Medicine Co., Ltd, 11. MacroGenics, 16. Gilead Sciences, 18. AstraZeneca/MedImmune LLC, 19. Recombio SL, 20. Regeneron Pharmaceuticals, 21. Innovent Biologics (Suzhou) Co. Ltd., 22. BeiGene, 24. Biocad, 25. Novartis, 26. Philogen SpA, 27. Tracon.

TABLE 5

| | Molecular | | |
|---|---|---|---|
| INN or code name | format | Target | Late-stage study indication(s) |
| I-131-BC8, Iomab-B¹ | Murine IgG1, | CD45 | Ablation of bone marrow prior to |
| | radiolabeled | | hematopoietic cell transplantation |
| | | | in AML patients |
| Isatuximab² | Humanized* | CD38 | Multiple myeloma |
| | IgG1 | | |
| Ublituximab³ | Chimeric IgG1 | CD20 | Chronic lymphocytic |
| | | | Leukemia, non-Hodgkin lymphoma, |
| | | | multiple sclerosis |
| XMAB-5574, MOR208⁵ | Humanized IgG1 | CD19 | Diffuse large B-cell lymphoma |
| Utomilumab⁶ | Human IgG2 | 4-1BB | Diffuse large B-cell lymphoma |
| | | (CD137) | |
| Oportuzumab monatox⁸ | Humanized scFv | EpCAM | Bladder cancer |
| | immunotoxin | | |
| Camrelizumab¹⁰ | Humanized IgG4 | PD-1 | Hepatocellular carcinoma, esophageal |
| | | | carcinoma |
| Margetuximab¹¹ | Chimeric IgG1 | HER2 | Breast cancer |
| Andecaliximab¹⁶ | Humanized* | MMP-9 | Gastric cancer or gastroesophageal junction |
| | IgG4 | | adenocarcinoma |
| Tremelimumab¹⁸ | Human IgG2 | CTLA-4 | Non-small cell lung, head & neck, |
| | | | urothelial cancer, hepatocellular carcinoma |
| Racotumomab¹⁹ | Murine IgG1 | NGcGM3 | Non-small cell lung cancer |
| Cemiplimab²⁰ | Human mAb | PD-1 | Cutaneous squamous cell carcinoma; non- |
| | | | small cell |
| | | | lung cancer, cervical cancer |
| IBI308²¹ | Human mAb | PD-1 | Squamous cell non-small cell |
| | | | lung cancer |
| BGB-A317²² | Humanized mAb | PD-1 | Non-small cell |
| | | | lung cancer |
| BCD-100²⁴ | Human mAb | PD-1 | Melanoma |
| PDR001²⁵ | Humanized IgG4 | PD-1 | Melanoma |
| L19IL2/L19TNF²⁶ | scFv immuno- | Fibronectin | Melanoma |
| | conjugates | extra- | |
| | | domain B | |
| Carotuximab²⁷ | Chimeric IgG1 | Endoglin | Soft tissue sarcoma, angiosarcoma, renal |
| | | | cell carcinoma, wet age related macular |
| | | | degeneration |

Additional exemplary antibodies and fusion proteins to be expressed by ceDNA vectors include, but are not limited to those described below:

Brodalumab (SILIQ®, LUMICEF®, KYNTHEUM®, AMG-827) is a human IgG2 antibody that targets the IL-17 receptor A (IL-17RA) and prevents inflammatory signaling of IL-17A, IL-17F and IL-17C pro-inflammatory cytokines through IL-17RA. Brodalumab is approved in the US (SILIQ®), the EU (KYNTHEUM®), and Japan (LUMICEF®). Brodalumab is indicated for the treatment of adult patients with moderate to severe plaque psoriasis, who are candidates for systemic therapy or phototherapy and who have failed to respond, or have stopped responding to other systemic therapies.

Dupilumab (DUPIXENT®, REGN668/SAR231893) is a human IgG4 mAb that targets IL-4 receptor (IL4R), thus blocking inflammatory responses mediated by IL-4 and IL-13. The mAb was approved in the US and EU for patients with atopic dermatitis.

Ocrelizumab (OCREVUS®) is a humanized IgG1 antibody targeting CD20-positive B cells. Such B cells play a role in myelin damage and multiple sclerosis pathogenesis.

Ocrelizumab was granted an approval for the treatment of relapsing multiple sclerosis (RMS) and primary progressive multiple sclerosis (PPMS) in the US.

Sarilumab (KEVZARA®, SAR153191, REGN88) is a human IgG1 antibody targeting IL-6 receptor (IL-6R), and was approved in Canada, the US and EU for patients with moderately to severely active rheumatoid arthritis (RA) who had an inadequate response or intolerance to one or more disease modifying anti-rheumatic drugs (DMARDs), such as methotrexate (MTX).

Benralizumab (FASENRA®, MEDI-563) is an afucosylated IgG1 mAb targeting the α-subunit of IL-5R found on eosinophils, received FDA approval for the add-on maintenance treatment of patients with severe asthma aged 12 years and older.

Emicizumab (HEMLIBRA®, emicizumab-kxwh, ACE910, RO5534262) is a bispecific IgG4 mAb targeting Factor IXa and X, was approved by FDA. The drug was approved to prevent or reduce the frequency of bleeding episodes in adult and pediatric patients with hemophilia A who have developed Factor VIII inhibitors. As of Dec. 1, 2017, a total of 9 antibody therapeutics were undergoing regulatory review in either the US or EU. Of these, 8 (ibalizumab, burosumab, tildrakizumab, caplacizumab, erenumab, fremanezumab, galcanezumab, romosozumab) have not yet received marketing approval. Mogamulizumab was granted a first global approval in Japan on Mar. 20, 2012.

Ibalizumab is an IgG4 mAb targeting CD4, is being evaluated by the FDA as a treatment for multi-drug resistant human immunodeficiency virus (HIV) infection.

Burosumab (KRN23) is a human IgG1 mAb targeting fibroblast growth factor 23 (FGF23), a hormone that regulates phosphate excretion and active vitamin D production by the kidney.

Tildrakizumab (SCH 900222/MK-3222) is a humanized IgG1 mAb targeting IL-23p19. Marketing applications for tildrakizumab as a treatment for moderate to severe plaque psoriasis have been submitted in the EU and US.

Caplacizumab (ALX-0081) is a bivalent single-domain antibody (Nanobody®) targeting von Willebrand factor and is undergoing regulatory review as a treatment for acquired thrombotic thrombocytopenic purpura (aTTP), a rare, life-threatening blood clotting disorder involving the formation of microclots that lead to low platelet counts, tissue ischemia and organ dysfunction in aTTP patients.

Erenumab (AIMOVIG™, AMG 334) is an IgG2 mAb that targets the receptor for calcitonin gene-related peptide (CGRP), which is involved in the development of sensitized nociceptive neurons. Marketing applications for erenumab for the prevention of migraine in patients experiencing four or more migraine days per month were submitted in the EU and US.

Fremanezumab (TEV-48125) is an IgG2 mAb targeting CGRP that is undergoing regulatory review for the preventive treatment of migraine.

Galcanezumab (LY2951742) is an IgG4 mAb targeting CGRP that is undergoing regulatory review for prevention of episodic and chronic migraine in adults.

Romosozumab (EVENITY™, AMG785) is a humanized IgG2 mAb targeting sclerostin, is being evaluated as a treatment for osteoporosis in women and men.

Mogamulizumab (KW-0761, POTELIGEo®) is an IgG1 afucosylated humanized mAb targeting CC chemokine receptor 4 (CCR4) expressed on tumor cells of patients with cutaneous T cell leukemia lymphoma (CTCL), including mycosis fungoides and Sézary syndrome.

Lanadelumab (SHP643, DX-2930) is a human IgG1 mAb that targets plasma kallikrein and thereby prevents production of bradykinin.

Crizanlizumab (SEG101) is a humanized mAb targeting P-selectin, also known as CD62 and is undergoing evaluation as a treatment for sickle cell-related pain crises (SCPC), which are caused by vaso-occlusion in sickle cell disease patients.

Ravulizumab (ALXN1210) is a humanized mAb targeting complement component 5 (C5) that is undergoing evaluation in two Phase 3 studies of patients with paroxysmal nocturnal hemoglobinuria (PNH).

Eptinezumab (ALD403) is an IgG1 mAb targeting calcitonin gene-related peptide (CGRP) and is being evaluated for migraine prevention.

Risankizumab (ABBV066, BI655066) is an IgG1 mAb targeting the p19 subunit of IL-23, which has been implicated in the pathogenesis of psoriasis.

Satralizumab (SA237) is a humanized IgG2 targeting IL-6R, is undergoing evaluation in two Phase 3 studies of patients with neuromyelitis optica (NMO) or NMO spectrum disorder.

Brolucizumab (RTH258) is a single-chain variable fragment (scFv) targeting vascular endothelial growth factor (VEGF)-A.

PRO140 is a humanized IgG4 mAb, blocks the human immunodeficiency virus (HIV) co-receptor CCR5 on T cells, thereby preventing viral entry.

Lampalizumab (RG7417, FCFD4514S) is a humanized antigen-binding fragment (Fab), inhibits activation and amplification of the alternative complement pathway by binding complement factor D.

Roledumab (LFB-R593) is a human IgG1 anti-rhesus (Rh)D mAb derived from LFB S.A.'s EMABLING® technology platform, which alters fucosylation, leading to more effective binding of antibodies to effector cells. The antibody is designed to prevent some fetomaternal alloimmunization conditions, i.e., in RhD-negative pregnant women carrying an RhD-positive fetus.

Fasinumab (REGN475) is a human IgG4 mAb targeting nerve growth factor, is being evaluated in numerous late-stage studies as a treatment for moderate-to-severe osteoarthritis pain of the hip or knee, and chronic low back pain.

Etrolizumab (RH7413) is a humanized mAb that binds the p7 subunit of a407 and aEp7 integrin heterodimers, thereby inhibiting interactions with their ligands MAdCAM-1 and E-cadherin, respectively.

NEOD001 is a humanized IgG1 mAb, targets soluble and insoluble light chain aggregates that cause amyloid light chain (AL) amyloidosis, a disorder characterized by excessive accumulation of protein aggregates in the tissues and organs, including the heart, kidneys and liver.

Gantenerumab (RO4909832) is a human mAb targeting fibrillar amyloid-β that is undergoing investigation as a treatment for Alzheimer's disease.

Anifrolumab (MEDI-546) is a human IgG1 mAb targeting type-I interferon (IFN) receptor subunit 1 that is being evaluated as a treatment for SLE.

Moxetumomab pasudotox (HA22, CAT-8015) is a recombinant immunotoxin containing the 38-kDa cytotoxic portion of Pseudomonas exotoxin A fused to an antibody variable fragment targeting CD22.

Cemiplimab (REGN2810, SAR439684), a human antibody that targets programmed death-1 (PD1), is undergoing evaluation as a treatment for metastatic or unresectable cutaneous squamous cell carcinoma (CSCC).

Ublituximab (LFB-R603, TGT-1101, TGTX-1101) is a glycoengineered chimeric mAb targeting CD20.

Tremelimumab (CP-675,206) is a human IgG2 antibody targeting the cytotoxic T lymphocyte-associated antigen-4 (CTLA4). In physiological conditions, CD28 interacts with B7 ligands (CD80, CD86), leading to T cell activation, proliferation and CTLA-4 upregulation. CTLA-4 binds B7 ligands with higher affinity than CD28 and terminates T-cell responses.

Isatuximab (SAR650984) is an anti-CD38 IgG1 chimeric antibody being evaluated for treatment of patients with relapsed and refractory multiple myeloma (MM).

BCD-100 is a human antibody targeting programmed cell death-1 (PD-1)

Carotuximab (TRC105) is a chimeric IgG1 antibody targeting endoglin (CD105), a protein highly expressed on angiogenic and proliferative endothelial cells. The mAb binds human CD105 on proliferating endothelium with a KD of 1-2 ng/mL and induces ADCC of human umbilical vein endothelial cells.

Camrelizumab (INCSHR-1210, SHR-1210) is an IgG4u humanized antibody targeting PD-1.

Glembatumumab vedotin (CDX-011, CR011-vcMMAE) is an IgG2 human antibody targeting transmembrane glycoprotein non-metastatic gene B (gpNMB) conjugated to monomethyl auristatin E, a cytotoxic drug that, when released in cancer cells, may lead to tumor cell death.

Mirvetuximab soravtansine (IMGN853) is an antibody targeting folate receptor alpha (FRa) that is conjugated to 3-4 molecules of the maytansinoid drug DM4, an anti-mitotic agent.

Oportuzumab monatox (VICINIUM™, VB4-845) is an anti-epithelial cell adhesion molecule (EpCAM) recombinant humanized antibody scFv fragment conjugated to Pseudomonas aeruginosa exotoxin A.

L19IL2/L19TNF (DAROMUN) is a fusion protein composed of the scFv of L19 antibody, which targets the extradomain B of fibronectin, fused to either human IL2 or human TNF.

Further additional exemplary antibodies and fusion proteins can be selected from any of the following: benralizumab, MEDI-8968, anifrolumab, MEDI7183, sifalimunab, MEDI-575, tralokinunab from AstraZeneca and MedImmnune; BAN2401 from Biogen Idec/Eisai Co. LTD (“Eisai”)/BioArctic Neuroscience AB; CDP7657 an anti-CD40L monovalent pegylated Fab antibody fragment, STX-100 an anti-avB6 mAb, BIIB059, Anti-TWEAK (BIIB023), and BIIB022 from Biogen; fulranumab from Janssen and Amgen; BI-204/RG7418 from BioInvent International/Genentech; BT-062 (indatuximab ravtansine) from Biotest Pharmaceuticals Corporation; XmAb from Boehringer Ingelheim/Xencor; anti-IP10 from Bristol-Myers Squibb; J 591 Lu-177 from BZL Biologics LLC; CDX-011 (glembatumumab vedotin), CDX-0401 from Celldex Therapeutics; foravirumab from Crucell; tigatuzumab from Daiichi Sankyo Company Limited; MORAb-404, MORAb-009 (amatuximab) from Eisai; LY2382770 from Eli Lilly; DI17E6 from EMD Serono Inc; zanolimumab from Emergent BioSolutions, Inc.; FG-3019 from FibroGen. Inc.; catumaxomab from Fresenius SE & Co. KGaA; pateclizumab, rontalizumab from Genentech; fresolimumab from Genzyme & Sanofi; GS-6624 (simtuzumab) from Gilead; CNTO-328, bapineuzumab (AAB-001), carlumab, CNTO-136 from Janssen; KB003 from KaloBios Pharmaceuticals, Inc.; ASKP1240 from Kyowa; RN-307 from Labtys Biologics Inc.; ecromeximab from Life Science Pharmaceuticals; LY2495655. LY2928057, LY3015014. LY2951742 from Eli Lilly; MBL-HCV1 from MassBiologics; AME-133v from MENTRIK Biotech, LLC; abituzumab from Merck KGaA; MM-121 from Merrimack Pharmaceuticals, Inc.; MCS 110, QAX576. QBX258, QGE031 from Novartis AG; HCD122 from Novartis AG and XOMA Corporation (“XOMA”); NN8555 from Novo Nordisk; bavituximab, cotara from Peregrine Pharmaceuticals, Inc.; PSMA-ADC from Progenies Pharmaceuticals, Inc.; oregovomab from Quest Pharmatech, Inc.; fasinumab (REGN475), REGN1033, SAR231893, REGN846 from Regeneron; RG7160, CIM331, RG7745 from Roche; ibalizumab (TMB-355) from TaiMed Biologics Inc.; TCN-032 from Theraclone Sciences; TRC105 from TRACON Pharmaceuticals. Inc.; UB-421 from United Biomedical Inc.; VB4-845 from Viventia Bio, Inc.; ABT-110 from AbbVie: Caplacizumab, Ozoralizumab from Ablynx; PRO 140 from CytoDyn, Inc.; GS-CDA1, MDX-1388 from Medarex. Inc.; AMG 827, AMG 888 from Amgen; ublituximab from TG Therapeutics Inc.; TOL101 from Tolera Therapeutics, Inc.; huN901-DMI (lorvotuzumab mertansine) from ImmunoGen Inc.; eprauzumab Y-90/veltuzumab combination (LMMU-102) from Immunomedics, Inc.; anti-fibrin mAb/3B6/22 Tc-99m from Agenix, Limited; ALD403 from Alder Biopharmaceuticals. Inc.; RN6G/PF-04382923 from Pfizer; CG201 from CG Therapeuties, Inc.; KBOO1-A from KaloBios Pharmaceuticals/Sanofi; KRN-23 from Kyowa; Y-90 hPAM 4 from Immunomedics, Inc.; Tarextumab from Morphosys AG & OncoMed Pharmaceuticals. Inc.; LFG316 from Morphosys AG & Novartis AG: CNTO3157, CNTO6785 from Morphosys AG & Jannsen; RG6013 from Roche & Chugai; MM-111 from Merrimack Pharmaceuticals, Inc. (“Merrimack”); GSK2862277 from GlaxoSmithKline; AMG 282, AMG 172, AMG 595, AMG 745, AMG 761 from Amgen; BVX-20 from Biocon; CT-P19, CT-P24, CT-P25, CT-P26, CT-P27, CT-P4 from Celltrion; GSK284933, GSK2398852, GSK2618960, GSK1223249, GSK933776A from GlaxoSmithKline; anetumab ravtansine from Morphosys AG & Bayer AG; BI-836845 from Morphosys AG & Boehringer Ingelheim; NOV-7, NOV-8 from Morphosys AG & Novartis AG; MM-302, MM-310, MM-141, MM-131, MM-151 from Merrimack, RG7882 from Roche & Seattle Genetics; RG7841 from Roche/Genentech; PF-06410293, PF-06438179, PF-06439535, PF-04605412, PF-05280586 from Pfizer; RG7716, RG7936, gentenerumab, RG7444 from Roche; MEDI-547, MEDI-565, MEDI1814, MEDI4920, MEDI8897, MEDI-4212, MEDI-5117, MEDI-7814 from Astrazeneca; ulocuplumab, PCSK9 adnectin from Bristol-Myers Squibb; FPA009, FPA145 from FivePrime Therapeutics, Inc.; GS-5745 from Gilead; BIW-8962, KHK4083, KHK6640 from Kyowa Hakko Kirin; MM-141 from Merck KGaA; REGN1154, REGN1193, REGN1400, REGN1500, REGN1908-1909, REGN2009, REGN2176-3, REGN728 from Regeneron; SAR307746 from Sanofi; SGN-CD70A from Seattle Genetics; ALX-0141, ALX40171 from Ablynx; milatuzumab-DOX, milatuzumab, TF2, from Immunomedics, Inc.; MLN0264 from Millennium; ABT-981 from AbbVie; AbGn-168H from AbGenomics International Inc.; ficlatuzumab from AVEO; BI-505 from BioInvent International; CDX-1127, CDX-301 from Celldex Therapeutics; CLT-008 from Cellerant Therapeutics Inc.; VGX-100 from Circadian; U3-1565 from Daiichi Sankyo Company Limited; DKN-01 from Dekkun Corp.; flanvotumab (TYRP1 protein), IL-1 β antibody, IMC-CS4 from Eli Lilly; VEGFR3 mAb, IMC-TR1 (LY3022859) from Eli Lilly and ImClone, LLC; Anthim from Elusys Therapeutics Inc.; HuL2G7 from Galaxy Biotech LLC; IMGB853, IMGN529 from ImmunoGen Inc.; CNTO-5, CNTO-5825 from Janssen; KD-247 from Kaketsuken; KB004 from KaloBios Pharmaceuticals; MGA271, MGAH22 from MacroGenics. Inc.; XmAb5574 from MorphoSys AG/Xencor; ensituximab (NPC-1C) from Neogenix Oncology, Inc.; LFA102 from Novartis AG and XOMA; AT1355 from Novartis AG; SAN-300 from Santarus Inc.; SelG1 from Selexys; HuM195/rGel from Targa Therapeutics. Corp.; VX15 from Teva Pharmaceuticals, Industries Ltd. (“Teva”) and Vaccinex Inc.; TCN-202 from Theraclone Sciences; XmAb2513, XmAb5872 from Xencor; XOMA 3AB from XOMA and National Institute for Allergy and Infectious Diseases; neuroblastoma antibody vaccine from MabVax Therapeutics; Cytolin from CytoDyn, Inc.; Thravixa from Emergent BioSolutions Inc.; and FB 301 from Cytovance Biologics; rabies mAb from Janssen and Sanofi; flu mAb from Janssen and partly funded by National Institutes of Health; MB-003 and ZMapp from Mapp Biopharmaceutical, Inc.; and ZMAb from Defyrus IncG

### III. ceDNA Vector in General for Use in Production of Antibodies and Fusion Proteins

Embodiments of the invention are based on methods and compositions comprising close ended linear duplexed (ceDNA) vectors that can express a transgene (e.g., an antibody or fusion protein). In some embodiments, the transgene is a sequence encoding an antibody or fusion protein. The ceDNA vectors for antibody or fusion protein production as described herein are not limited by size, thereby permitting, for example, expression of all of the components necessary for expression of a transgene from a single vector. The ceDNA vector for antibody or fusion protein production is preferably duplex, e.g. self-complementary, over at least a portion of the molecule, such as the expression cassette (e.g. ceDNA is not a double stranded circular molecule). The ceDNA vector has covalently closed ends, and thus is resistant to exonuclease digestion (e.g. exonuclease I or exonuclease III), e.g. for over an hour at 37° C.

In general, a ceDNA vector for antibody or fusion protein production as disclosed herein, comprises in the 5′ to 3′ direction: a first adeno-associated virus (AAV) inverted terminal repeat (ITR), a nucleotide sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

Encompassed herein are methods and compositions comprising the ceDNA vector for antibody or fusion protein production, which may further include a delivery system, such as but not limited to, a liposome nanoparticle delivery system. Non-limiting exemplary liposome nanoparticle systems encompassed for use are disclosed herein. In some aspects, the disclosure provides for a lipid nanoparticle comprising ceDNA and an ionizable lipid. For example, a lipid nanoparticle formulation that is made and loaded with a ceDNA vector obtained by the process is disclosed in International Application PCT/US2018/050042, filed on Sep. 7, 2018, which is incorporated herein.

The ceDNA vectors for antibody or fusion protein production as disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

FIG. 1A-1E show schematics of non-limiting, exemplary ceDNA vectors for antibody or fusion protein production, or the corresponding sequence of ceDNA plasmids. ceDNA vectors for antibody or fusion protein production are capsid-free and can be obtained from a plasmid encoding in this order: a first ITR, an expression cassette comprising a transgene and a second ITR. The expression cassette may include one or more regulatory sequences that allows and/or controls the expression of the transgene, e.g., where the expression cassette can comprise one or more of, in this order: an enhancer/promoter, an ORF reporter (transgene), a post-transcription regulatory element (e.g., WPRE), and a polyadenylation and termination signal (e.g., BGH polyA).

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene, e.g., antibody or fusion protein. In some embodiments, the ceDNA vector comprises additional components to regulate expression of the transgene, for example, a regulatory switch, which are described herein in the section entitled “Regulatory Switches” for controlling and regulating the expression of the antibody or fusion protein, and can include if desired, a regulatory switch which is a kill switch to enable controlled cell death of a cell comprising a ceDNA vector.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene in the range of 500 to 50,000 nucleotides in length. In some embodiments, the expression cassette can comprise a transgene in the range of 500 to 75,000 nucleotides in length. In some embodiments, the expression cassette can comprise a transgene which is in the range of 500 to 10,000 nucleotides in length. In some embodiments, the expression cassette can comprise a transgene which is in the range of 1000 to 10,000 nucleotides in length. In some embodiments, the expression cassette can comprise a transgene which is in the range of 500 to 5,000 nucleotides in length. The ceDNA vectors do not have the size limitations of encapsidated AAV vectors, thus enable delivery of a large-size expression cassette to provide efficient transgene expression. In some embodiments, the ceDNA vector is devoid of prokaryote-specific methylation.

ceDNA expression cassette can include, for example, an expressible exogenous sequence (e.g., open reading frame) or transgene that encodes a protein that is either absent, inactive, or insufficient activity in the recipient subject or a gene that encodes a protein having a desired biological or a therapeutic effect. The transgene can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure.

The expression cassette can comprise any transgene (e.g., encoding an antibody or fusion protein), for example, an antibody or fusion protein useful for treating a disease or disorder in a subject, i.e., a therapeutic antibody or fusion protein. A ceDNA vector can be used to deliver and express any antibody or fusion protein of interest in the subject, alone or in combination with nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. Preferably a ceDNA vector disclosed herein is used for therapeutic purposes (e.g., for medical, diagnostic, or veterinary uses) or immunogenic polypeptides. In certain embodiments, a ceDNA vector is useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, micro-RNAs, and their antisense counterparts (e.g., antagoMiR)), antibodies, fusion proteins, or any combination thereof.

The expression cassette can also encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Sequences provided in the expression cassette, expression construct of a ceDNA vector for antibody or fusion protein production described herein can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database. In some embodiments, the nucleic acid encoding the antibody or fusion protein is optimized for human expression, and/or is a human antibody or humanized antibody, or antigen-binding fragment thereof, as known in the art.

In some embodiments, an antibody or fusion protein expressed by the ceDNA vector is a therapeutic antibody or fusion protein, including therapeutic activating antibodies or fusion proteins or therapeutic neutralizing (e.g., blocking or inhibitory) antibodies or fusion proteins.

A transgene expressed by the ceDNA vector for antibody or fusion protein production as disclosed herein encodes an antibody or fusion protein. Antibodies and fusion proteins are well known in the art and can bind to any protein of interest, including, but not limited to, a ligand, a receptor, a toxin, a hormone, an enzyme, or a cell surface protein, or pathogen or viral protein or antigen, as well as pre- and post-translationally modified proteins, such as glycoproteins or SUMOylated proteins (e.g., ant-SUMO2/3 antibody) etc. Antibodies also include antibodies that bind to any antigen, including but not limited to nucleic acids, e.g., DNA (e.g., anti-dsDNA antibodies), RNA (e.g., anti-RNA binding antibodies). In some embodiments, the antibodies or fusion proteins produced by the ceDNA vectors disclosed herein are neutralizing antibodies or fusion proteins. Exemplary genes to be targeted and proteins of interest are described in detail in the methods of use and methods of treatment sections herein. Antibodies, fusion proteins, as well as variants, and/or active fragments thereof, for use in the treatment, prophylaxis, and/or amelioration of one or more symptoms of a disease, dysfunction, injury, and/or disorder are encompassed for use in the ceDNA vectors for antibody or fusion protein production disclosed herein. Exemplary therapeutic genes are described herein in the section entitled “Method of Treatment”.

There are many structural features of ceDNA vectors for antibody or fusion protein production that differ from plasmid-based expression vectors. ceDNA vectors may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a non-limiting example in a promoter or enhancer region. Another important feature distinguishing ceDNA vectors from plasmid expression vectors is that ceDNA vectors are single-strand linear DNA having closed ends, while plasmids are always double-strand DNA.

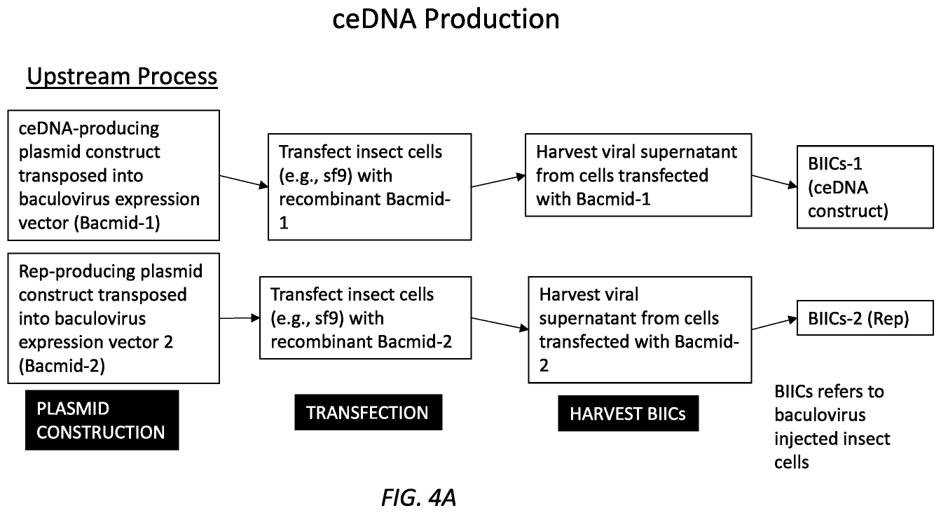

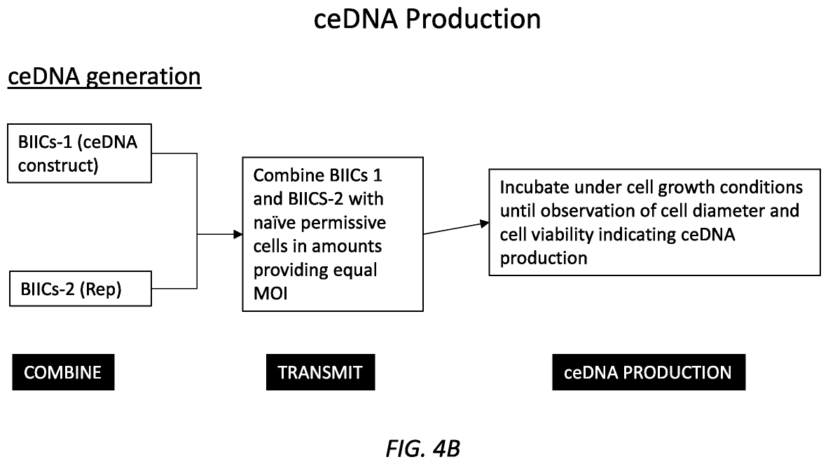

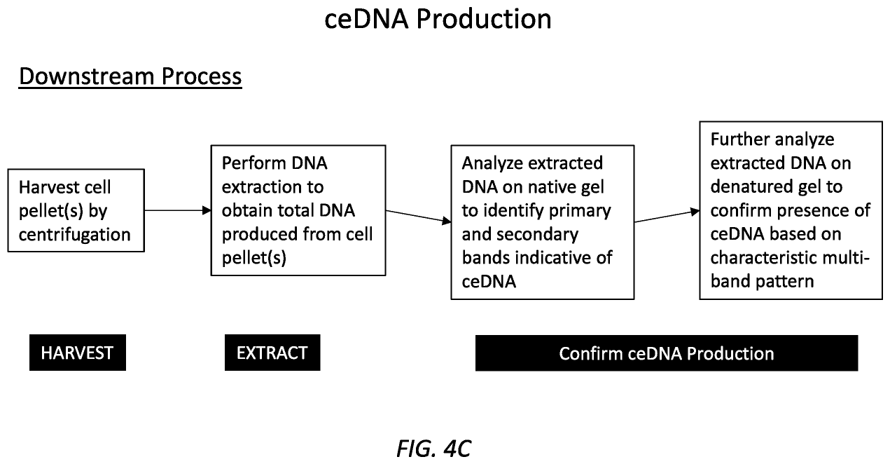

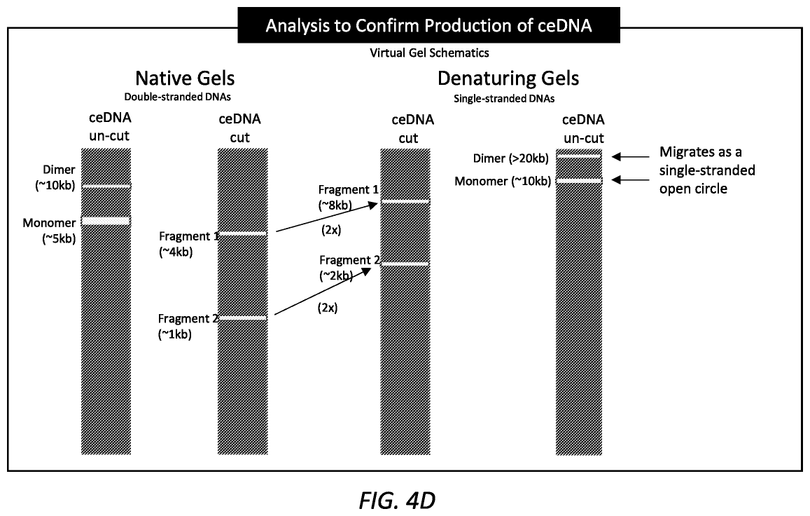

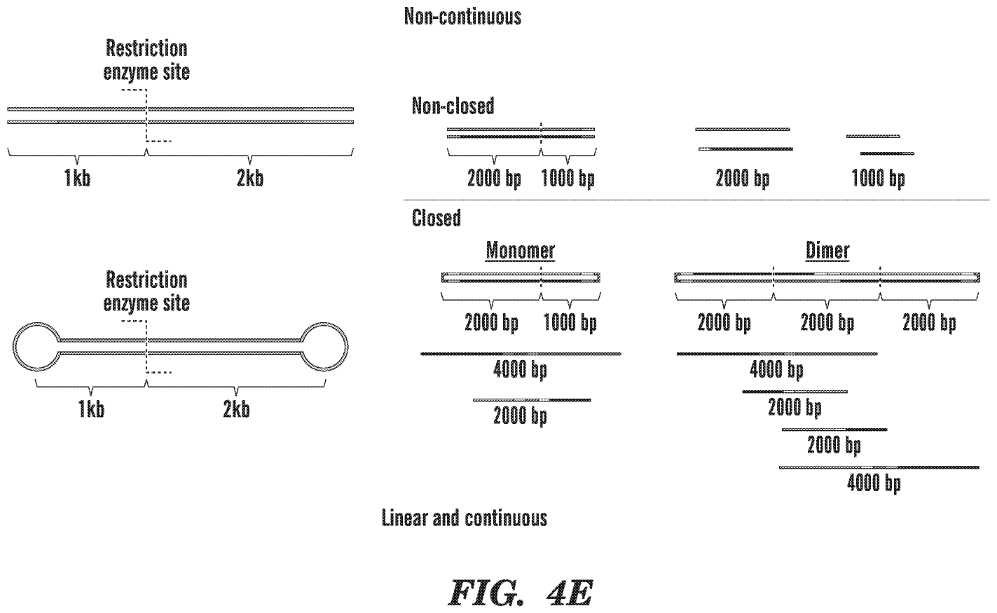

ceDNA vectors for antibody or fusion protein production produced by the methods provided herein preferably have a linear and continuous structure rather than a non-continuous structure, as determined by restriction enzyme digestion assay (FIG. 4D). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several advantages of using a ceDNA vector for antibody or fusion protein production as described herein over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ (SEQ ID NO: 60) for AAV2) and a terminal resolution site (TRS; 5′-AGTTGG-3′ (SEQ ID NO: 64) for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### IV. ITRs

As disclosed herein, ceDNA vectors for antibody or fusion protein production contain a transgene or heterologous nucleic acid sequence positioned between two inverted terminal repeat (ITR) sequences, where the ITR sequences can be an asymmetrical ITR pair or a symmetrical- or substantially symmetrical ITR pair, as these terms are defined herein. A ceDNA vector as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). In some embodiments, the 5′ WT-ITR can be from one serotype and the 3′ WT-ITR from a different serotype, as discussed herein.

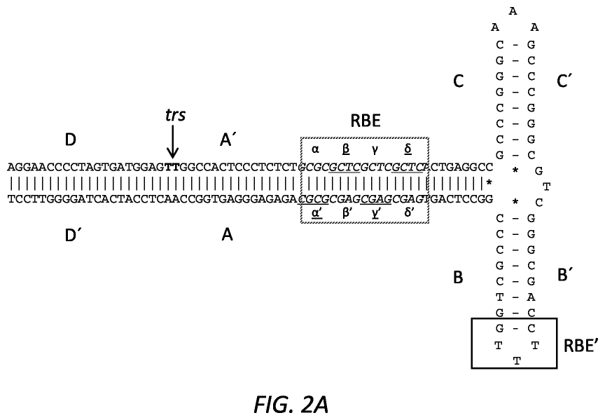

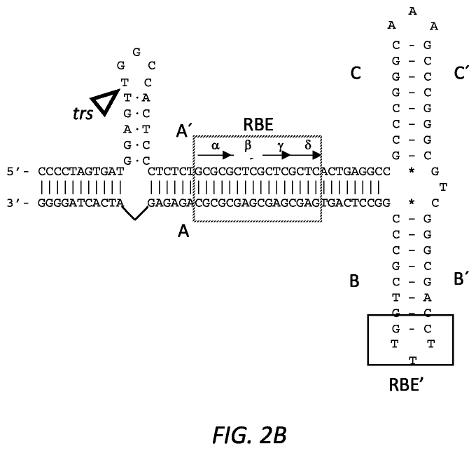

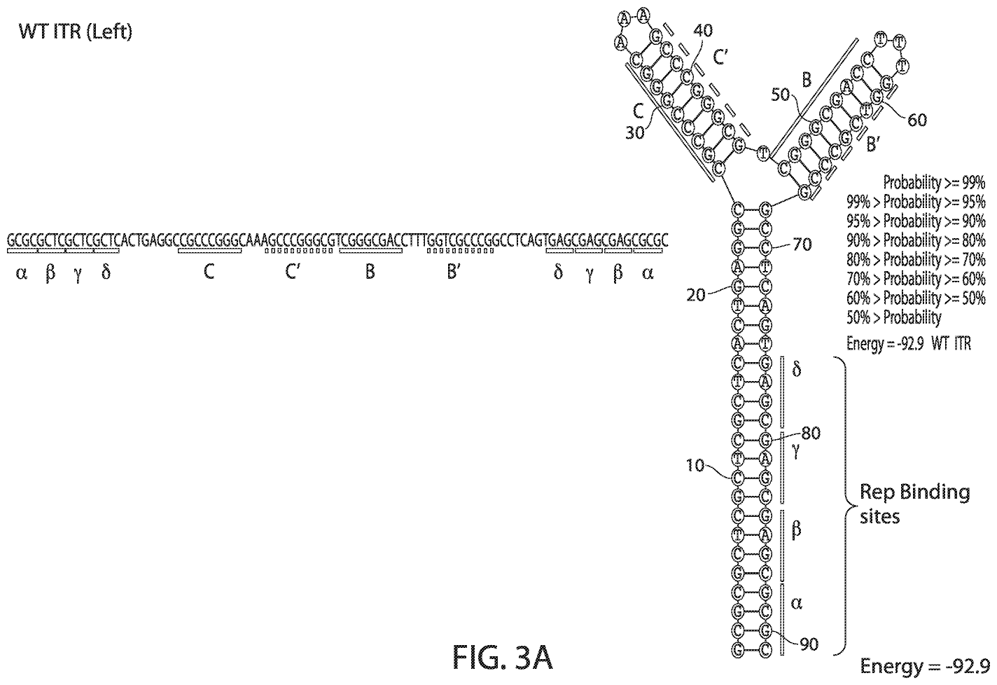

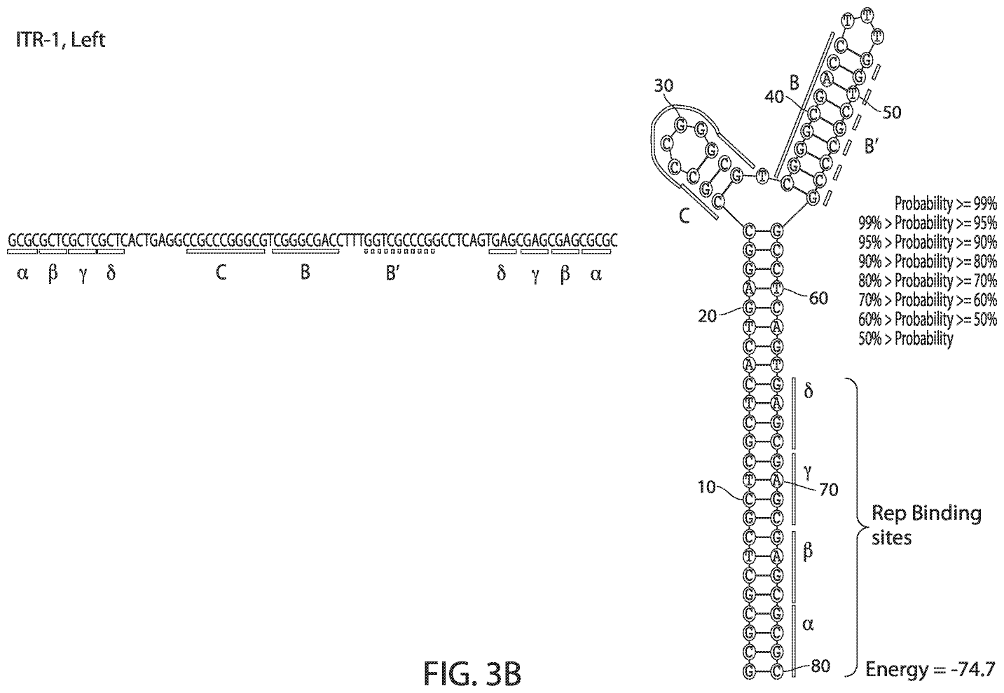

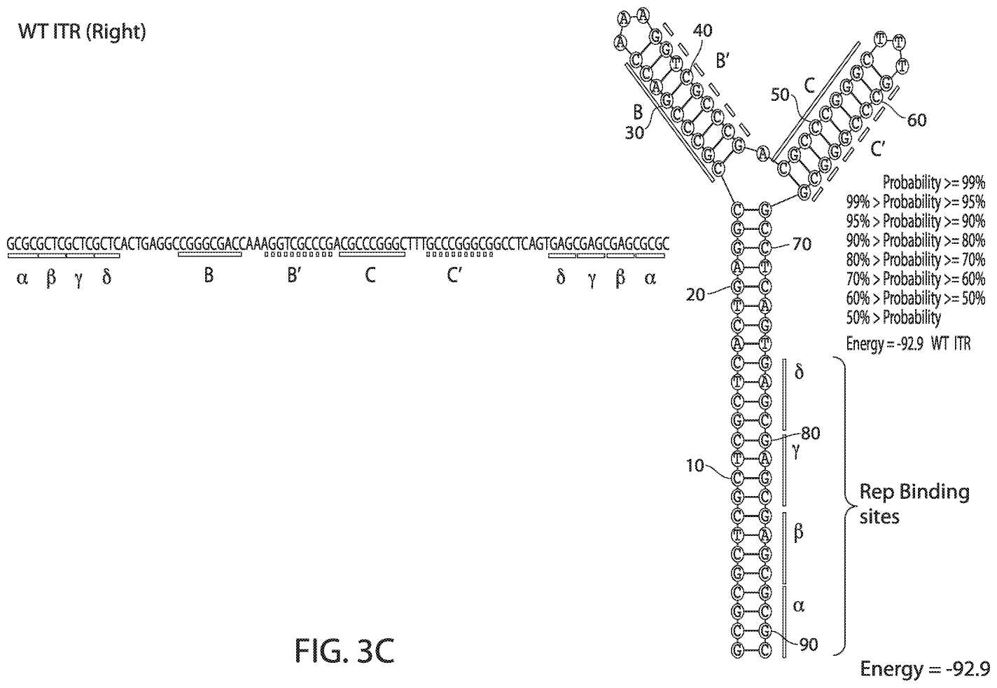

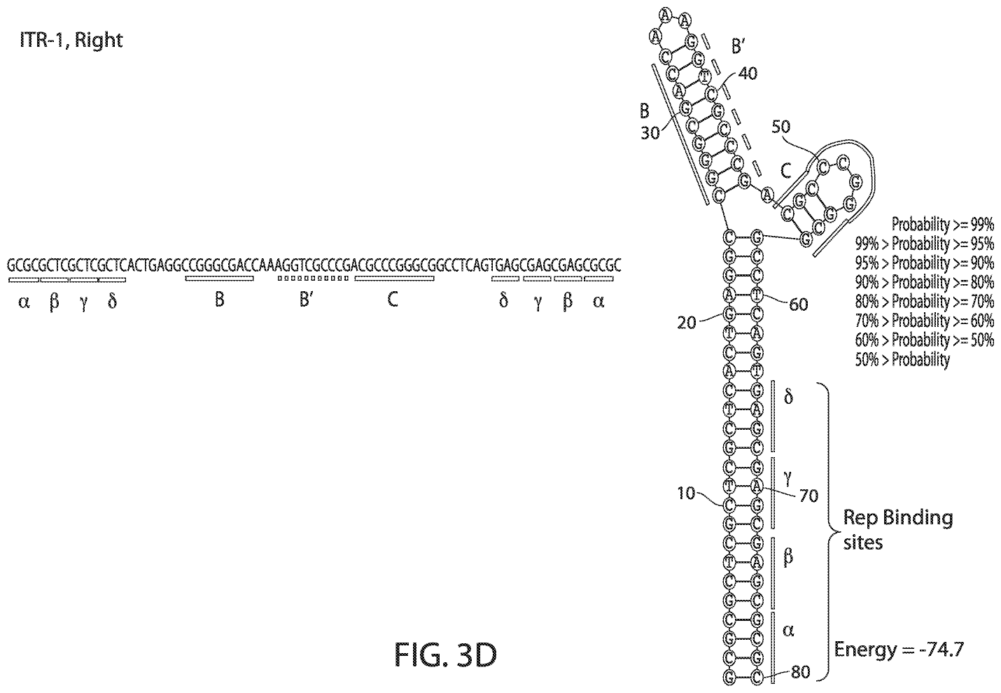

An ordinarily skilled artisan is aware that ITR sequences have a common structure of a double-stranded Holliday junction, which typically is a T-shaped or Y-shaped hairpin structure (see e.g., FIG. 2A and FIG. 3A), where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

### A. Symmetrical ITR Pairs

In some embodiments, a ceDNA vector for antibody or fusion protein production as described herein comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

(i) Wildtype ITRs

In some embodiments, the symmetrical ITRs, or substantially symmetrical ITRs are wild type (WT-ITRs) as described herein. That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, ceDNA vectors contain a transgene or heterologous nucleic acid sequence positioned between two flanking wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. In some embodiments, a wild-type ITR sequence (e.g. AAV WT-ITR) comprises a functional Rep binding site (RBS; e.g. 5′-GCGCGCTCGCTCGCTC-3′ for AAV2, SEQ ID NO: 60) and a functional terminal resolution site (TRS; e.g. 5′-AGTT-3′, SEQ ID NO: 62).

In one aspect, ceDNA vectors for antibody or fusion protein production are obtainable from a vector polynucleotide that encodes a heterologous nucleic acid operatively positioned between two WT inverted terminal repeat sequences (WT-ITRs) (e.g. AAV WT-ITRs). That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. In some embodiments, the 5′ WT-ITR is from one AAV serotype, and the 3′ WT-ITR is from the same or a different AAV serotype. In some embodiments, the 5′ WT-ITR and the 3′WT-ITR are mirror images of each other, that is they are symmetrical. In some embodiments, the 5′ WT-ITR and the 3′ WT-ITR are from the same AAV serotype.

WT ITRs are well known. In one embodiment the two ITRs are from the same AAV2 serotype. In certain embodiments one can use WT from other serotypes. There are a number of serotypes that are homologous, e.g. AAV2, AAV4, AAV6, AAV8. In one embodiment, closely homologous ITRs (e.g. ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA, e.g., the expression of the encoded antibody or fusion protein.

In some embodiments, one aspect of the technology described herein relates to a ceDNA vector for antibody or fusion protein production, wherein the ceDNA vector comprises at least one heterologous nucleotide sequence, operably positioned between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A, C-C′ and B-B′ loops in 3D space). In some embodiments, the symmetric WT-ITRs comprises a functional terminal resolution site and a Rep binding site. In some embodiments, the heterologous nucleic acid sequence encodes a transgene, and wherein the vector is not in a viral capsid.

In some embodiments, the WT-ITRs are the same but the reverse complement of each other. For example, the sequence AACG in the 5′ ITR may be CGTT (i.e., the reverse complement) in the 3′ ITR at the corresponding site. In one example, the 5′ WT-ITR sense strand comprises the sequence of ATCGATCG and the corresponding 3′ WT-ITR sense strand comprises CGATCGAT (i.e., the reverse complement of ATCGATCG). In some embodiments, the WT-ITRs ceDNA further comprises a terminal resolution site and a replication protein binding site (RPS) (sometimes referred to as a replicative protein binding site), e.g. a Rep binding site.

Exemplary WT-TTR sequences for use in the ceDNA vectors for antibody or fusion protein production comprising WT-ITRs are shown in Table 7 herein, which shows pairs of WT-ITRs (5′ WT-ITR and the 3′ WT-ITR).

As an exemplary example, the present disclosure provides a ceDNA vector for antibody or fusion protein production comprising a promoter operably linked to a transgene (e.g., heterologous nucleic acid sequence), with or without the regulatory switch, where the ceDNA is devoid of capsid proteins and is: (a) produced from a ceDNA-plasmid (e.g., see FIGS. 1F-1G) that encodes WT-ITRs, where each WT-ITR has the same number of intramolecularly duplexed base pairs in its hairpin secondary configuration (preferably excluding deletion of any AAA or TIT terminal loop in this configuration compared to these reference sequences), and (b) is identified as ceDNA using the assay for the identification of ceDNA by agarose gel electrophoresis under native gel and denaturing conditions in Example 1.

In some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g. AAV1, 3, 4, 5, 6, 7, 8, 9, 10, 11, and 12. In some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. In some embodiments, such a combination of WT ITRs is the combination of WT-ITRs from AAV2 and AAV6. In one embodiment, the substantially symmetrical WT-ITRs are when one is inverted relative to the other ITR at least 90% identical, at least 95% identical, at least 96% . . . 97% . . . 98% . . . 99% . . . 99.5% and all points in between, and has the same symmetrical three-dimensional spatial organization. In some embodiments, a WT-ITR pair are substantially symmetrical as they have symmetrical three-dimensional spatial organization, e.g., have the same 3D organization of the A, C-C′, B-B′ and D arms. In one embodiment, a substantially symmetrical WT-ITR pair are inverted relative to the other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs). In some embodiments, a substantially symmetrical WT-ITR pair are inverted relative to each other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 6 indicates exemplary combinations of WT-ITRs.

Table 6: Exemplary combinations of WT-ITRs from the same serotype or different serotypes, or different parvoviruses. The order shown is not indicative of the ITR position, for example, “AAV1, AAV2” demonstrates that the ceDNA can comprise a WT-AAV1 ITR in the 5′ position, and a WT-AAV2 TR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh10, AAV-DJ, and AAV-DJ8 genome (E.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 parvoviris (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 6

| | | | | |
|---|---|---|---|---|
| AAV1, AAV1 | AAV2, AAV2 | AAV3, AAV3 | AAV4, AAV4 | AAV5, AAV5 |
| AAV1, AAV2 | AAV2, AAV3 | AAV3, AAV4 | AAV4, AAV5 | AAV5, AAV6 |
| AAV1, AAV3 | AAV2, AAV4 | AAV3, AAV5 | AAV4, AAV6 | AAV5, AAV7 |
| AAV1, AAV4 | AAV2, AAV5 | AAV3, AAV6 | AAV4, AAV7 | AAV5, AAV8 |
| AAV1, AAV5 | AAV2, AAV6 | AAV3, AAV7 | AAV4, AAV8 | AAV5, AAV9 |
| AAV1, AAV6 | AAV2, AAV7 | AAV3, AAV8 | AAV4, AAV9 | AAV5, AAV10 |
| AAV1, AAV7 | AAV2, AAV8 | AAV3, AAV9 | AAV4, AAV10 | AAV5, AAV11 |
| AAV1, AAV8 | AAV2, AAV9 | AAV3, AAV10 | AAV4, AAV11 | AAV5, AAV12 |
| AAV1, AAV9 | AAV2, AAV10 | AAV3, AAV11 | AAV4, AAV12 | AAV5, AAVRH8 |
| AAV1, AAV10 | AAV2, AAV11 | AAV3, AAV12 | AAV4, AAVRH8 | AAV5, AAVRH10 |
| AAV1, AAV11 | AAV2, AAV12 | AAV3, AAVRH8 | AAV4, AAVRH10 | AAV5, AAV13 |
| AAV1, AAV12 | AAV2, AAVRH8 | AAV3, AAVRH10 | AAV4, AAV13 | AAV5, AAVDJ |
| AAV1, AAVRH8 | AAV2, AAVRH10 | AAV3, AAV13 | AAV4, AAVDJ | AAV5, AAVDJ8 |
| AAV1, AAVRH10 | AAV2, AAV13 | AAV3, AAVDJ | AAV4, AAVDJ8 | AAV5, AVIAN |
| AAV1, AAV13 | AAV2, AAVDJ | AAV3, AAVDJ8 | AAV4, AVIAN | AAV5, BOVINE |
| AAV1, AAVDJ | AAV2, AAVDJ8 | AAV3, AVIAN | AAV4, BOVINE | AAV5, CANINE |
| AAV1, AAVDJ8 | AAV2, AVIAN | AAV3, BOVINE | AAV4, CANINE | AAV5, EQUINE |
| AAV1, AVIAN | AAV2, BOVINE | AAV3, CANINE | AAV4, EQUINE | AAV5, GOAT |
| AAV1, BOVINE | AAV2, CANINE | AAV3, EQUINE | AAV4, GOAT | AAV5, SHRIMP |
| AAV1, CANINE | AAV2, EQUINE | AAV3, GOAT | AAV4, SHRIMP | AAV5, PORCINE |
| AAV1, EQUINE | AAV2, GOAT | AAV3, SHRIMP | AAV4, PORCINE | AAV5, INSECT |
| AAV1, GOAT | AAV2, SHRIMP | AAV3, PORCINE | AAV4, INSECT | AAV5, OVINE |
| AAV1, SHRIMP | AAV2, PORCINE | AAV3, INSECT | AAV4, OVINE | AAV5, B19 |
| AAV1, PORCINE | AAV2, INSECT | AAV3, OVINE | AAV4, B19 | AAV5, MVM |
| AAV1, INSECT | AAV2, OVINE | AAV3, B19 | AAV4, MVM | AAV5, GOOSE |
| AAV1, OVINE | AAV2, B19 | AAV3, MVM | AAV4, GOOSE | AAV5, SNAKE |
| AAV1, B19 | AAV2, MVM | AAV3, GOOSE | AAV4, SNAKE | |
| AAV1, MVM | AAV2, GOOSE | AAV3, SNAKE | | |
| AAV1, GOOSE | AAV2, SNAKE | | | |
| AAV1, SNAKE | | | | |
| AAV6, AAV6 | AAV7, AAV7 | AAV8, AAV8 | AAV9, AAV9 | AAV10, AAV10 |
| AAV6, AAV7 | AAV7, AAV8 | AAV8, AAV9 | AAV9, AAV10 | AAV10, AAV11 |
| AAV6, AAV8 | AAV7, AAV9 | AAV8, AAV10 | AAV9, AAV11 | AAV10, AAV12 |
| AAV6, AAV9 | AAV7, AAV10 | AAV8, AAV11 | AAV9, AAV12 | AAV10, AAVRH8 |
| AAV6, AAV10 | AAV7, AAV11 | AAV8, AAV12 | AAV9, AAVRH8 | AAV10, AAVRH10 |
| AAV6, AAV11 | AAV7, AAV12 | AAV8, AAVRH8 | AAV9, AAVRH10 | AAV10, AAV13 |
| AAV6, AAV12 | AAV7, AAVRH8 | AAV8, AAVRH10 | AAV9, AAV13 | AAV10, AAVDJ |
| AAV6, AAVRH8 | AAV7, AAVRH10 | AAV8, AAV13 | AAV9, AAVDJ | AAV10, AAVDJ8 |
| AAV6, AAVRH10 | AAV7, AAV13 | AAV8, AAVDJ | AAV9, AAVDJ8 | AAV10, AVIAN |
| AAV6, AAV13 | AAV7, AAVDJ | AAV8, AAVDJ8 | AAV9, AVIAN | AAV10, BOVINE |
| AAV6, AAVDJ | AAV7, AAVDJ8 | AAV8, AVIAN | AAV9, BOVINE | AAV10, CANINE |
| AAV6, AAVDJ8 | AAV7, AVIAN | AAV8, BOVINE | AAV9, CANINE | AAV10, EQUINE |
| AAV6, AVIAN | AAV7, BOVINE | AAV8, CANINE | AAV9, EQUINE | AAV10, GOAT |
| AAV6, BOVINE | AAV7, CANINE | AAV8, EQUINE | AAV9, GOAT | AAV10, SHRIMP |
| AAV6, CANINE | AAV7, EQUINE | AAV8, GOAT | AAV9, SHRIMP | AAV10, PORCINE |
| AAV6, EQUINE | AAV7, GOAT | AAV8, SHRIMP | AAV9, PORCINE | AAV10, INSECT |
| AAV6, GOAT | AAV7, SHRIMP | AAV8, PORCINE | AAV9, INSECT | AAV10, OVINE |
| AAV6, SHRIMP | AAV7, PORCINE | AAV8, INSECT | AAV9, OVINE | AAV10, B19 |
| AAV6, PORCINE | AAV7, INSECT | AAV8, OVINE | AAV9, B19 | AAV10, MVM |
| AAV6, INSECT | AAV7, OVINE | AAV8, B19 | AAV9, MVM | AAV10, GOOSE |
| AAV6, OVINE | AAV7, B19 | AAV8, MVM | AAV9, GOOSE | AAV10, SNAKE |
| AAV6, B19 | AAV7, MVM | AAV8, GOOSE | AAV9, SNAKE | |
| AAV6, MVM | AAV7, GOOSE | AAV8, SNAKE | | |
| AAV6, GOOSE | AAV7, SNAKE | | | |
| AAV6, SNAKE | | | | |
| AAV11, AAV11 | AAV12, AAV12 | AAVRH8, AAVRH8 | AAVRH10, AAVRH10 | AAV13, AAV13 |
| AAV11, AAV12 | AAV12, AAVRH8 | AAVRH8, AAVRH10 | AAVRH10, AAV13 | AAV13, AAVDJ |
| AAV11, AAVRH8 | AAV12, AAVRH10 | AAVRH8, AAV13 | AAVRH10, AAVDJ | AAV13, AAVDJ8 |
| AAV11, AAVRH10 | AAV12, AAV13 | AAVRH8, AAVDJ | AAVRH10, AAVDJ8 | AAV13, AVIAN |
| AAV11, AAV13 | AAV12, AAVDJ | AAVRH8, AAVDJ8 | AAVRH10, AVIAN | AAV13, BOVINE |
| AAV11, AAVDJ | AAV12, AAVDJ8 | AAVRH8, AVIAN | AAVRH10, BOVINE | AAV13, CANINE |
| AAV11, AAVDJ8 | AAV12, AVIAN | AAVRH8, BOVINE | AAVRH10, CANINE | AAV13, EQUINE |
| AAV11, AVIAN | AAV12, BOVINE | AAVRH8, CANINE | AAVRH10, EQUINE | AAV13, GOAT |
| AAV11, BOVINE | AAV12, CANINE | AAVRH8, EQUINE | AAVRH10, GOAT | AAV13, SHRIMP |
| AAV11, CANINE | AAV12, EQUINE | AAVRH8, GOAT | AAVRH10, SHRIMP | AAV13, PORCINE |
| AAV11, EQUINE | AAV12, GOAT | AAVRH8, SHRIMP | AAVRH10, PORCINE | AAV13, INSECT |
| AAV11, GOAT | AAV12, SHRIMP | AAVRH8, PORCINE | AAVRH10, INSECT | AAV13, OVINE |
| AAV11, SHRIMP | AAV12, PORCINE | AAVRH8, INSECT | AAVRH10, OVINE | AAV13, B19 |
| AAV11, PORCINE | AAV12, INSECT | AAVRH8, OVINE | AAVRH10, B19 | AAV13, MVM |
| AAV11, INSECT | AAV12, OVINE | AAVRH8, B19 | AAVRH10, MVM | AAV13, GOOSE |
| AAV11, OVINE | AAV12, B19 | AAVRH8, MVM | AAVRH10, GOOSE | AAV13, SNAKE |
| AAV11, B19 | AAV12, MVM | AAVRH8, GOOSE | AAVRH10, SNAKE | |
| AAV11, MVM | AAV12, GOOSE | AAVRH8, SNAKE | | |
| AAV11, GOOSE | AAV12, SNAKE | | | |
| AAV11, SNAKE | | | | |
| AAVDJ, AAVDJ | AAVDJ8, AVVDJ8 | AVIAN, AVIAN | BOVINE, BOVINE | CANINE, CANINE |
| AAVDJ, AAVDJ8 | AAVDJ8, AVIAN | AVIAN, BOVINE | BOVINE, CANINE | CANINE, EQUINE |
| AAVDJ, AVIAN | AAVDJ8, BOVINE | AVIAN, CANINE | BOVINE, EQUINE | CANINE, GOAT |
| AAVDJ, BOVINE | AAVDJ8, CANINE | AVIAN, EQUINE | BOVINE, GOAT | CANINE, SHRIMP |
| AAVDJ, CANINE | AAVDJ8, EQUINE | AVIAN, GOAT | BOVINE, SHRIMP | CANINE, PORCINE |
| AAVDJ, EQUINE | AAVDJ8, GOAT | AVIAN, SHRIMP | BOVINE, PORCINE | CANINE, INSECT |
| AAVDJ, GOAT | AAVDJ8, SHRIMP | AVIAN, PORCINE | BOVINE, INSECT | CANINE, OVINE |
| AAVDJ, SHRIMP | AAVDJ8, PORCINE | AVIAN, INSECT | BOVINE, OVINE | CANINE, B19 |
| AAVDJ, PORCINE | AAVDJ8, INSECT | AVIAN, OVINE | BOVINE, B19 | CANINE, MVM |
| AAVDJ, INSECT | AAVDJ8, OVINE | AVIAN, B19 | BOVINE, MVM | CANINE, GOOSE |
| AAVDJ, OVINE | AAVDJ8, B19 | AVIAN, MVM | BOVINE, GOOSE | CANINE, SNAKE |
| AAVDJ, B19 | AAVDJ8, MVM | AVIAN, GOOSE | BOVINE, SNAKE | |
| AAVDJ, MVM | AAVDJ8, GOOSE | AVIAN, SNAKE | | |
| AAVDJ, GOOSE | AAVDJ8, SNAKE | | | |
| AAVDJ, SNAKE | | | | |
| EQUINE, EQUINE | GOAT, GOAT | SHRIMP, SHRIMP | PORCINE, PORCINE | INSECT, INSECT |
| EQUINE, GOAT | GOAT, SHRIMP | SHRIMP, PORCINE | PORCINE, INSECT | INSECT, OVINE |
| EQUINE, SHRIMP | GOAT, PORCINE | SHRIMP, INSECT | PORCINE, OVINE | INSECT, B19 |
| EQUINE, PORCINE | GOAT, INSECT | SHRIMP, OVINE | PORCINE, B19 | INSECT, MVM |
| EQUINE, INSECT | GOAT, OVINE | SHRIMP, B19 | PORCINE, MVM | INSECT, GOOSE |
| EQUINE, OVINE | GOAT, B19 | SHRIMP, MVM | PORCINE, GOOSE | INSECT, SNAKE |
| EQUINE, B19 | GOAT, MVM | SHRIMP, GOOSE | PORCINE, SNAKE | |
| EQUINE, MVM | GOAT, GOOSE | SHRIMP, SNAKE | | |
| EQUINE, GOOSE | GOAT, SNAKE | | | |
| EQUINE, SNAKE | | | | |
| OVINE, OVINE | B19, B19 | MVM, MVM | GOOSE, GOOSE | SNAKE, SNAKE |
| OVINE, B19 | B19, MVM | MVM, GOOSE | GOOSE, SNAKE | |
| OVINE, MVM | B19, GOOSE | MVM, SNAKE | | |
| OVINE, GOOSE | B19, SNAKE | | | |
| OVINE, SNAKE | | | | |

By way of example only, Table 7 shows the sequences of exemplary WT-ITRs from some different AAV serotypes.

TABLE 7

| AAV | 5′ WT-ITR | 3′ WT-ITR |
|---|---|---|
| serotype | (LEFT) | (RIGHT) |
| AAV1 | 5′-TTGCCCACTCCC | 5′-TTACCCTAGTGATG |
| | TCTCTGCGCGCTCGC | GAGTTGCCCACTCCCTC |
| | TCGCTCGGTGGGGCC | TCTGCGCGCGTCGCTCG |
| | TGCGGACCAAAGGTC | CTCGGTGGGGCCGGCAG |
| | CGCAGACGGCAGAGG | AGGAGACCTCTGCCGTC |
| | TCTCCTCTGCCGGCC | TGCGGACCTTTGGTCCG |
| | CCACCGAGCGAGCGA | CAGGCCCCACCGAGCGA |
| | CGCGCGCAGAGAGGG | GCGAGCGCGCAGAGAGG |
| | AGTGGGCAACTCCAT | GAGTGGGCAA-3′ |
| | CACTAGGGTAA-3′ | (SEQ ID NO: 10) |
| | (SEQ ID NO: 5) | |
| AAV2 | CCTGCAGGCAGCTGC | AGGAACCCCTAGTGATG |
| | GCGCTCGCTCGCTCA | GAGTTGGCCACTCCCTC |
| | CTGAGGCCGCCCGGG | TCTGCGCGCTCGCTCGC |
| | CAAAGCCCGGGCGTC | TCACTGAGGCCGGGCGA |
| | GGGCGACCTTTGGTC | CCAAAGGTCGCCCGACG |
| | GCCCGGCCTCAGTGA | CCCGGGCTTTGCCCGGG |
| | GCGAGCGAGCGCGCA | CGGCCTCAGTGAGCGAG |
| | GAGAGGGAGTGGCCA | CGAGCGCGCAGCTGCCT |
| | ACTCCATCACTAGGG | GCAGG |
| | GTTCCT | (SEQ ID NO: 1) |
| | (SEQ ID NO: 2) | |
| AAV3 | 5′-TTGGCCACTCCC | 5′-ATACCTCTAGTGAT |
| | TCTATGCGCACTCGC | GGAGTTGGCCACTCCCT |
| | TCGCTCGGTGGGGCC | CTATGCGCACTCGCTCG |
| | TGGCGACCAAAGGTC | CTCGGTGGGGCCGGACG |
| | GCCAGACGGACGTGG | TGGAAACCCACGTCCGT |
| | GTTTCCACGTCCGGC | CTGGCGACCTTTGGTCG |
| | CCCACCGAGCGAGCG | CCAGGCCCCACCGAGCG |
| | AGTGCGCATAGAGGG | AGCGAGTGCGCATAGAG |
| | AGTGGCCAACTCCAT | GGAGTGGCCAA-3′ |
| | CACTAGAGGTAT-3′ | (SEQ ID NO: 11) |
| | (SEQ ID NO: 6) | |
| AAV4 | 5′-TTGGCCACTCCC | 5′-AGTTGGCCACATTA |
| | TCTATGCGCGCTCGC | GCTATGCGCGCTCGCTC |
| | TCACTCACTCGGCCC | ACTCACTCGGCCCTGGA |
| | TGGAGACCAAAGGTC | GACCAAAGGTCTCCAGA |
| | TCCAGACTGCCGGCC | CTGCCGGCCTCTGGCCG |
| | TCTGGCCGGCAGGGC | GCAGGGCCGAGTGAGTG |
| | CGAGTGAGTGAGCGA | AGCGAGCGCGCATAGAG |
| | GCGCGCATAGAGGGA | GGAGTGGCCAA-3′ |
| | GTGGCCAACT-3′ | (SEQ ID NO: 12) |
| | (SEQ ID NO: 7) | |
| AAV5 | 5′-TCCCCCCTGTCG | 5′-CTTACAAAACCCCC |
| | CGTTCGCTCGCTCGC | TTGCTTGAGAGTGTGGC |
| | TGGCTCGTTTGGGGG | ACTCTCCCCCCTGTCGC |
| | GGCGACGGCCAGAGG | GTTCGCTCGCTCGCTGG |
| | GCCGTCGTCTGGCAG | CTCGTTTGGGGGGGTGG |
| | CTCTTTGAGCTGCCA | CAGCTCAAAGAGCTGCC |
| | CCCCCCCAAACGAGC | AGACGACGGCCCTCTGG |
| | CAGCGAGCGAGCGAA | CCGTCGCCCCCCCAAAC |
| | CGCGACAGGGGGGAG | GAGCCAGCGAGCGAGCG |
| | AGTGCCACACTCTCA | AACGCGACAGGGGGGA- |
| | AGCAAGGGG | 3′ |
| | GTTTTGTAAG -3′ | (SEQ ID NO: 13) |
| | (SEQ ID NO: 8) | |
| AAV6 | 5′-TTGCCCACTCCC | 5′-ATACCCCTAGTGAT |
| | TCTAATGCGCGCTCG | GGAGTTGCCCACTCCCT |
| | CTCGCTCGGTGGGGC | CTATGCGCGCTCGCTCG |
| | CTGCGGACCAAAGGT | CTCGGTGGGGCCGGCAG |
| | CCGCAGACGGCAGAG | AGGAGACCTCTGCCGTC |
| | GTCTCCTCTGCCGGC | TGCGGACCTTTGGTCCG |
| | CCCACCGAGCGAGCG | CAGGCCCCACCGAGCGA |
| | AGCGCGCATAGAGGG | GCGAGCGCGCATTAGAG |
| | AGTGGGCAACTCCAT | GGAGTGGGCAA |
| | CACTAGGGGTAT-3′ | (SEQ ID NO: 14) |
| | (SEQ ID NO: 9) | |

In some embodiments, the nucleotide sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present invention, the ceDNA vector for antibody or fusion protein production does not have a WT-ITR consisting of the nucleotide sequence selected from any of: SEQ ID NOs: 1, 2, 5-14. In alternative embodiments of the present invention, if a ceDNA vector has a WT-ITR comprising the nucleotide sequence selected from any of: SEQ ID NOs: 1, 2, 5-14, then the flanking ITR is also WT and the ceDNA vector comprises a regulatory switch, e.g., as disclosed herein and in International application PCT/US18/49996 (e.g., see Table 11 of PCT/US18/49996). In some embodiments, the ceDNA vector for antibody or fusion protein production comprises a regulatory switch as disclosed herein and a WT-ITR selected having the nucleotide sequence selected from any of the group consisting of: SEQ ID NO: 1, 2, 5-14.

The ceDNA vector for antibody or fusion protein production as described herein can include WT-ITR structures that retains an operable RBE, trs and RBE′ portion. FIG. 2A and FIG. 2B, using wild-type ITRs for exemplary purposes, show one possible mechanism for the operation of a trs site within a wild type ITR structure portion of a ceDNA vector. In some embodiments, the ceDNA vector for antibody or fusion protein production contains one or more functional WT-ITR polynucleotide sequences that comprise a Rep-binding site (RBS; 5′-GCGCGCTCGCTCGCTC-3′ (SEQ ID NO: 60) for AAV2) and a terminal resolution site (TRS; 5′-AGTT (SEQ ID NO: 62)). In some embodiments, at least one WT-ITR is functional. In alternative embodiments, where a ceDNA vector for antibody or fusion protein production comprises two WT-ITRs that are substantially symmetrical to each other, at least one WT-ITR is functional and at least one WT-ITR is non-functional.

B. Modified ITR (Mod-ITR) in General for ceDNA Vectors Comprising Asymmetric ITR Pairs or Symmetric ITR Pairs

As discussed herein, a ceDNA vector for antibody or fusion protein production can comprise a symmetrical ITR pair or an asymmetrical ITR pair. In both instances, one or both of the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′ and B-B′ arms).

In some embodiments, a modified ITR is an ITRs that is modified by deletion, insertion, and/or substitution as compared to a wild-type ITR sequence (e.g. AAV ITR). In some embodiments, at least one of the ITRs in the ceDNA vector comprises a functional Rep binding site (RBS; e.g. 5′-GCGCGCTCGCTCGCTC-3′ for AAV2, SEQ ID NO: 60) and a functional terminal resolution site (TRS; e.g. 5′-AGTT-3′, SEQ ID NO: 62.) In one embodiment, at least one of the ITRs is a non-functional ITR. In one embodiment, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In some embodiments, a mod-ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects, a synthetic ITR may interact preferentially with a wild type Rep or a Rep of a specific serotype, or in some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The invention further provides populations and pluralities of ceDNA vectors comprising mod-ITRs from a combination of different AAV serotypes—that is, one mod-ITR can be from one AAV serotype and the other mod-ITR can be from a different serotype. Without wishing to be bound by theory, in one embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR.

More specifically, the ability of a structural element to functionally interact with a particular large Rep protein can be altered by modifying the structural element. For example, the nucleotide sequence of the structural element can be modified as compared to the wild-type sequence of the ITR. In one embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 8 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. In some embodiments, any modification of at least one nucleotide (e.g., a deletion, insertion and/or substitution) in any of the regions of C and/or C′ and/or B and/or B′ retains three sequential T nucleotides (i.e., TIT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TIT) in at least one terminal loop. In some embodiments, a truncated C-C′ arm and/or a truncated B-B′ arm has three sequential T nucleotides (i.e., TTT) in the terminal loop.

TABLE 8
Exemplary combinations of modifications of at least one nucleotide
(e.g., a deletion, insertion and/or substitution) to different
B-B′ and C-C′ regions or arms of ITRs (X indicates
a nucleotide modification, e.g., addition, deletion or substitution
of at least one nucleotide in the region).

| B region | B′ region | C region | C′ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

In some embodiments, mod-ITR for use in a ceDNA vector for antibody or fusion protein production comprises an asymmetric ITR pair, or a symmetric mod-ITR pair as disclosed herein, can comprise any one of the combinations of modifications shown in Table 8, and also a modification of at least one nucleotide in any one or more of the regions selected from: between A′ and C, between C and C′, between C′ and B, between B and B′ and between B′ and A. In some embodiments, any modification of at least one nucleotide (e.g., a deletion, insertion and/or substitution) in the C or C′ or B or B′ regions, still preserves the terminal loop of the stem-loop. In some embodiments, any modification of at least one nucleotide (e.g., a deletion, insertion and/or substitution) between C and C′ and/or B and B′ retains three sequential T nucleotides (i.e., TT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop. In some embodiments, a modified ITR for use herein can comprise any one of the combinations of modifications shown in Table 8, and also a modification of at least one nucleotide (e.g., a deletion, insertion and/or substitution) in any one or more of the regions selected from: A′, A and/or D. For example, in some embodiments, a modified ITR for use herein can comprise any one of the combinations of modifications shown in Table 8, and also a modification of at least one nucleotide (e.g., a deletion, insertion and/or substitution) in the A region. In some embodiments, a modified ITR for use herein can comprise any one of the combinations of modifications shown in Table 8, and also a modification of at least one nucleotide (e.g., a deletion, insertion and/or substitution) in the A′ region. In some embodiments, a modified ITR for use herein can comprise any one of the combinations of modifications shown in Table 8, and also a modification of at least one nucleotide (e.g., a deletion, insertion and/or substitution) in the A and/or A′ region. In some embodiments, a modified ITR for use herein can comprise any one of the combinations of modifications shown in Table 8, and also a modification of at least one nucleotide (e.g., a deletion, insertion and/or substitution) in the D region.

In one embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. In one embodiment, the specific modifications to the ITRs are exemplified herein (e.g., SEQ ID NOS: 3, 4, 15-47, 101-116 or 165-187, or shown in FIG. 7A-7B of PCT/US2018/064242, filed on Dec. 6, 2018 (e.g., SEQ ID Nos 97-98, 101-103, 105-108, 111-112, 117-134, 545-54 in PCT/US2018/064242). In some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 3, 4, 15-47, 101-116 or 165-187, or the RBE-containing section of the A-A′ arm and C-C′ and B-B′ arms of SEQ ID NO: 3, 4, 15-47, 101-116 or 165-187, or shown in Tables 2-9 (i.e., SEQ ID NO: 110-112, 115-190, 200-468) of International application PCT/US18/49996, which is incorporated herein in its entirety by reference.

In some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-21 in FIG. 7A of PCT/US2018/064242, filed Dec. 6, 2018). In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A of PCT/US2018/064242, filed Dec. 6, 2018). In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 3B shows an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. In some embodiments, the modified ITR also comprises at least one base pair deletion from each of the B region and B′ regions, such that the B-B′ arm is also truncated relative to WT ITR.

In some embodiments, a modified ITR can have between 1 and 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. In some embodiments, a modified ITR can have between 1 and 30 nucleotide deletions relative to a full-length WT ITR sequence. In some embodiments, a modified ITR has between 2 and 20 nucleotide deletions relative to a full-length wild-type ITR sequence.

In some embodiments, a modified ITR does not contain any nucleotide deletions in the RBE-containing portion of the A or A′ regions, so as not to interfere with DNA replication (e.g. binding to an RBE by Rep protein, or nicking at a terminal resolution site). In some embodiments, a modified ITR encompassed for use herein has one or more deletions in the B, B′, C, and/or C region as described herein.

In some embodiments, a ceDNA vector for antibody or fusion protein production comprising a symmetric ITR pair or asymmetric ITR pair comprises a regulatory switch as disclosed herein and at least one modified ITR selected having the nucleotide sequence selected from any of the group consisting of: SEQ ID NO: 3, 4, 15-47, 101-116 or 165-187.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In one embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector for antibody or fusion protein production as described herein can include an ITR structure that is modified with respect to the wild type AAV2 ITR structure disclosed herein, but still retains an operable RBE, trs and RBE′ portion. FIG. 2A and FIG. 2B show one possible mechanism for the operation of a trs site within a wild type ITR structure portion of a ceDNA vector for antibody or fusion protein production. In some embodiments, the ceDNA vector for antibody or fusion protein production contains one or more functional ITR polynucleotide sequences that comprise a Rep-binding site (RBS; 5′-GCGCGCTCGCTCGCTC-3′ (SEQ ID NO: 60) for AAV2) and a terminal resolution site (TRS; 5′-AGTT (SEQ ID NO: 62)). In some embodiments, at least one ITR (wt or modified ITR) is functional. In alternative embodiments, where a ceDNA vector for antibody or fusion protein production comprises two modified ITRs that are different or asymmetrical to each other, at least one modified ITR is functional and at least one modified ITR is non-functional.

In some embodiments, the modified ITR (e.g., the left or right ITR) of a ceDNA vector for antibody or fusion protein production as described herein has modifications within the loop arm, the truncated arm, or the spacer. Exemplary sequences of ITRs having modifications within the loop arm, the truncated arm, or the spacer are listed in Table 2 (i.e., SEQ ID NOS: 135-190, 200-233); Table 3 (e.g., SEQ ID Nos: 234-263); Table 4 (e.g., SEQ ID NOs: 264-293); Table 5 (e.g., SEQ ID Nos: 294-318 herein); Table 6 (e.g., SEQ ID NO: 319-468; and Tables 7-9 (e.g., SEQ ID Nos: 101-110, 111-112, 115-134) or Table 10A or 10B (e.g., SEQ ID Nos: 9, 100, 469-483, 484-499) of International application PCT/US18/49996, which is incorporated herein in its entirety by reference.

In some embodiments, the modified ITR for use in a ceDNA vector for antibody or fusion protein production comprising an asymmetric ITR pair, or symmetric mod-ITR pair is selected from any or a combination of those shown in Tables 2, 3, 4, 5, 6, 7, 8, 9 and 10A-10B of International application PCT/US18/49996 which is incorporated herein in its entirety by reference.

Additional exemplary modified ITRs for use in a ceDNA vector for antibody or fusion protein production comprising an asymmetric ITR pair, or symmetric mod-ITR pair in each of the above classes are provided in Tables 9A and 9B. The predicted secondary structure of the Right modified ITRs in Table 9A are shown in FIG. 7A of International Application PCT/US2018/064242, filed Dec. 6, 2018, and the predicted secondary structure of the Left modified ITRs in Table 9B are shown in FIG. 7B of International Application PCT/US2018/064242, filed Dec. 6, 2018, which is incorporated herein in its entirety by reference.

Table 9A and Table 9B show exemplary right and left modified ITRs.

Table 9A: Exemplary modified right ITRs. These exemplary modified right ITRs can comprise the RBE of GCGCGCTCGCTCGCTC-3′ (SEQ ID NO: 60), spacer of ACTGAGGC (SEQ ID NO: 69), the spacer complement GCCTCAGT (SEQ ID NO: 70) and RBE′ (i.e., complement to RBE) of GAGCGAGCGAGCGCGC (SEQ ID NO: 71).

TABLE 9A
Exemplary Right modified ITRs

| | | SEQ |
|---|---|---|
| ITR | | ID |
| Construct | Sequence | NO: |
| ITR-18 | AGGAACCCCTAGTGATGGAGTTGGCCACTCCCT | 15 |
| Right | CTCTGCGCGCTCGCTCGCTCACTGAGGCGCACG | |
| | CCCGGGTTTCCCGGGCGGCCTCAGTGAGCGAGC | |
| | GAGCGCGCAGCTGCCTGCAGG | |
| ITR-19 | AGGAACCCCTAGTGATGGAGTTGGCCACTCCCT | 16 |
| Right | CTCTGCGCGCTCGCTCGCTCACTGAGGCCGACG | |
| | CCCGGGCTTTGCCCGGGCGGCCTCAGTGAGCGA | |
| | GCGAGCGCGCAGCTGCCTGCAGG | |
| ITR-20 | AGGAACCCCTAGTGATGGAGTTGGCCACTCCCT | 17 |
| Right | CTCTGCGCGCTCGCTCGCTCACTGAGGCCGGGC | |
| | GACCAAAGGTCGCCCGACGCCCGGGCGCCTCAG | |
| | TGAGCGAGCGAGCGCGCAGCTGCCTGCAGG | |
| ITR-21 | AGGAACCCCTAGTGATGGAGTTGGCCACTCCCT | 18 |
| Right | CTCTGCGCGCTCGCTCGCTCACTGAGGCTTTGC | |
| | CTCAGTGAGCGAGCGAGCGCGCAGCTGCCTGCA | |
| | GG | |
| ITR-22 | AGGAACCCCTAGTGATGGAGTTGGCCACTCCCT | 19 |
| Right | CTCTGCGCGCTCGCTCGCTCACTGAGGCCGGGC | |
| | GACAAAGTCGCCCGACGCCCGGGCTTTGCCCGG | |
| | GCGGCCTCAGTGAGCGAGCGAGCGCGCAGCTGC | |
| | CTGCAGG | |
| ITR-23 | AGGAACCCCTAGTGATGGAGTTGGCCACTCCCT | 20 |
| Right | CTCTGCGCGCTCGCTCGCTCACTGAGGCCGGGC | |
| | GAAAATCGCCCGACGCCCGGGCTTTGCCCGGGC | |
| | GGCCTCAGTGAGCGAGCGAGCGCGCAGCTGCCT | |
| | GCAGG | |
| ITR-24 | AGGAACCCCTAGTGATGGAGTTGGCCACTCCCT | 21 |
| Right | CTCTGCGCGCTCGCTCGCTCACTGAGGCCGGGC | |
| | GAAACGCCCGACGCCCGGGCTTTGCCCGGGCGG | |
| | CCTCAGTGAGCGAGCGAGCGCGCAGCTGCCTGC | |
| | AGG | |
| ITR-25 | AGGAACCCCTAGTGATGGAGTTGGCCACTCCCT | 22 |
| Right | CTCTGCGCGCTCGCTCGCTCACTGAGGCCGGGC | |
| | AAAGCCCGACGCCCGGGCTTTGCCCGGGCGGCC | |
| | TCAGTGAGCGAGCGAGCGCGCAGCTGCCTGCAG | |
| | G | |
| ITR-26 | AGGAACCCCTAGTGATGGAGTTGGCCACTCCCT | 23 |
| Right | CTCTGCGCGCTCGCTCGCTCACTGAGGCCGGGC | |
| | GACCAAAGGTCGCCCGACGCCCGGGTTTCCCGG | |
| | GCGGCCTCAGTGAGCGAGCGAGCGCGCAGCTGC | |
| | CTGCAGG | |
| ITR-27 | AGGAACCCCTAGTGATGGAGTTGGCCACTCCCT | 24 |
| Right | CTCTGCGCGCTCGCTCGCTCACTGAGGCCGGGC | |
| | GACCAAAGGTCGCCCGACGCCCGGTTTCCGGGC | |
| | GGCCTCAGTGAGCGAGCGAGCGCGCAGCTGCCT | |
| | GCAGG | |
| ITR-28 | AGGAACCCCTAGTGATGGAGTTGGCCACTCCCT | 25 |
| Right | CTCTGCGCGCTCGCTCGCTCACTGAGGCCGGGC | |
| | GACCAAAGGTCGCCCGACGCCCGTTTCGGGCGG | |
| | CCTCAGTGAGCGAGCGAGCGCGCAGCTGCCTGC | |
| | AGG | |
| ITR-29 | AGGAACCCCTAGTGATGGAGTTGGCCACTCCCT | 26 |
| Right | CTCTGCGCGCTCGCTCGCTCACTGAGGCCGGGC | |
| | GACCAAAGGTCGCCCGACGCCCTTTGGGCGGCC | |
| | TCAGTGAGCGAGCGAGCGCGCAGCTGCCTGCAG | |
| | G | |
| ITR-30 | AGGAACCCCTAGTGATGGAGTTGGCCACTCCCT | 27 |
| Right | CTCTGCGCGCTCGCTCGCTCACTGAGGCCGGGC | |
| | GACCAAAGGTCGCCCGACGCCTTTGGCGGCCTC | |
| | AGTGAGCGAGCGAGCGCGCAGCTGCCTGCAGG | |
| ITR-31 | AGGAACCCCTAGTGATGGAGTTGGCCACTCCCT | 28 |
| Right | GCTCTGCGCGCTCCTCGCTCACTGAGGCCGGGC | |
| | GACCAAAGGTCGCCCGACGCTTTGCGGCCTCAG | |
| | TGAGCGAGCGAGCGCGCAGCTGCCTGCAGG | |
| ITR-32 | AGGAACCCCTAGTGATGGAGTTGGCCACTCCCT | 29 |
| Right | CTCTGCGCGCTCGCTCGCTCACTGAGGCCGGGC | |
| | GACCAAAGGTCGCCCGACGTTTCGGCCTCAGTG | |
| | AGCGAGCGAGCGCGCAGCTGCCTGCAGG | |
| ITR-49 | AGGAACCCCTAGTGATGGAGTTGGCCACTCCCT | 30 |
| Right | CTCTGCGCGCTCGCTCGCTCACTGAGGCCGGGC | |
| | GACCAAAGGTCGCCCGACGGCCTCAGTGAGCGA | |
| | GCGAGCGCGCAGCTGCCTGCAGG | |
| ITR-50 | AGGAACCCCTAGTGATGGAGTTGGCCACTCCCT | 31 |
| right | CTCTGCGCGCTCGCTCGCTCACTGAGGCCGGGC | |
| | GACCAAAGGTCGCCCGACGCCCGGGCGGCCTCA | |
| | GTGAGCGAGCGAGCGCGCAGCTGCCTGCAGG | |

TABLE 9B: Exemplary modified left ITRs. These exemplary modified left ITRs can comprise the RBE of GCGCGCTCGCTCGCTC-3′ (SEQ ID NO: 60), spacer of ACTGAGGC (SEQ ID NO: 69), the spacer complement GCCTCAGT (SEQ ID NO: 70) and RBE complement (RBE′) of GAGCGAGCGAGCGCGC (SEQ ID NO: 71).

TABLE 9B
Exemplary modified left ITRs

| | | |
|---|---|---|
| ITR-33 | CCTGCAGGCAGCTGCGCGCTCGCTCGCTCACTGA | 32 |
| Left | GGCCGCCCGGGAAACCCGGGCGTGCGCCTCAGTG | |
| | AGCGAGCGAGCGCGCAGAGAGGGAGTGGCCAACT | |
| | CCATCACTAGGGGTTCCT | |
| ITR-34 | CCTGCAGGCAGCTGCGCGCTCGCTCGCTCACTGA | 33 |
| Left | GGCCGTCGGGCGACCTTTGGTCGCCCGGCCTCAG | |
| | TGAGCGAGCGAGCGCGCAGAGAGGGAGTGGCCAA | |
| | CTCCATCACTAGGGGTTCCT | |
| ITR-35 | CCTGCAGGCAGCTGCGCGCTCGCTCGCTCACTGA | 34 |
| Left | GGCCGCCCGGGCAAAGCCCGGGCGTCGGCCTCAG | |
| | TGAGCGAGCGAGCGCGCAGAGAGGGAGTGGCCAA | |
| | CTCCATCACTAGGGGTTCCT | |
| ITR-36 | CCTGCAGGCAGCTGCGCGCTCGCTCGCTCACTGA | 35 |
| Left | GGCGCCCGGGCGTCGGGCGACCTTTGGTCGCCCG | |
| | GCCTCAGTGAGCGAGCGAGCGCGCAGAGAGGGAG | |
| | TGGCCAACTCCATCACTAGGGGTTCCT | |
| ITR-37 | CCTGCAGGCAGCTGCGCGCTCGCTCGCTCACTGA | 36 |
| Left | GGCAAAGCCTCAGTGAGCGAGCGAGCGCGCAGAG | |
| | AGGGAGTGGCCAACTCCATCACTAGGGGTTCCT | |
| ITR-38 | CCTGCAGGCAGCTGCGCGCTCGCTCGCTCACTGA | 37 |
| Left | GGCCGCCCGGGCAAAGCCCGGGCGTCGGGCGACT | |
| | TTGTCGCCCGGCCTCAGTGAGCGAGCGAGCGCGC | |
| | AGAGAGGGAGTGGCCAACTCCATCACTAGGGGTT | |
| | CCT | |
| ITR-39 | CCTGCAGGCAGCTGCGCGCTCGCTCGCTCACTGA | 38 |
| Left | GGCCGCCCGGGCAAAGCCCGGGCGTCGGGCGATT | |
| | TTCGCCCGGCCTCAGTGAGCGAGCGAGCGCGCAG | |
| | AGAGGGAGTGGCCAACTCCATCACTAGGGGTTCC | |
| | T | |
| ITR-40 | CCTGCAGGCAGCTGCGCGCTCGCTCGCTCACTGA | 39 |
| Left | GGCCGCCCGGGCAAAGCCCGGGCGTCGGGCGTTT | |
| | CGCCCGGCCTCAGTGAGCGAGCGAGCGCGCAGAG | |
| | AGGGAGTGGCCAACTCCATCACTAGGGGTTCCT | |
| ITR-41 | CCTGCAGGCAGCTGCGCGCTCGCTCGCTCACTGA | 40 |
| Left | GGCCGCCCGGGCAAAGCCCGGGCGTCGGGCTTTG | |
| | CCCGGCCTCAGTGAGCGAGCGAGCGCGCAGAGAG | |
| | GGAGTGGCCAACTCCATCACTAGGGGTTCCT | |
| ITR-42 | CCTGCAGGCAGCTGCGCGCTCGCTCGCTCACTGA | 41 |
| Left | GGCCGCCCGGGAAACCCGGGCGTCGGGCGACCTT | |
| | TGGTCGCCCGGCCTCAGTGAGCGAGCGAGCGCGC | |
| | AGAGAGGGAGTGGCCAACTCCATCACTAGGGGTT | |
| | CCT | |
| ITR-43 | CCTGCAGGCAGCTGCGCGCTCGCTCGCTCACTGA | 42 |
| Left | GGCCGCCCGGAAACCGGGCGTCGGGCGACCTTTG | |
| | GTCGCCCGGCCTCAGTGAGCGAGCGAGCGCGCAG | |
| | AGAGGGAGTGGCCAACTCCATCACTAGGGGTTCC | |
| | T | |
| ITR-44 | CCTGCAGGCAGCTGCGCGCTCGCTCGCTCACTGA | 43 |
| Left | GGCCGCCCGAAACGGGCGTCGGGCGACCTTTGGT | |
| | CGCCCGGCCTCAGTGAGCGAGCGAGCGCGCAGAG | |
| | AGGGAGTGGCCAACTCCATCACTAGGGGTTCCT | |
| ITR-45 | CCTGCAGGCAGCTGCGCGCTCGCTCGCTCACTGA | 44 |
| Left | GGCCGCCCAAAGGGCGTCGGGCGACCTTTGGTCG | |
| | CCCGGCCTCAGTGAGCGAGCGAGCGCGCAGAGAG | |
| | GGAGTGGCCAACTCCATCACTAGGGGTTCCT | |
| ITR-46 | CCTGCAGGCAGCTGCGCGCTCGCTCGCTCACTGA | 45 |
| Left | GGCCGCCAAAGGCGTCGGGCGACCTTTGGTCGCC | |
| | CGGCCTCAGTGAGCGAGCGAGCGCGCAGAGAGGG | |
| | AGTGGCCAACTCCATCACTAGGGGTTCCT | |
| ITR-47 | CCTGCAGGCAGCTGCGCGCTCGCTCGCTCACTGA | 46 |
| Left | GGCCGCAAAGCGTCGGGCGACCTTTGGTCGCCCG | |
| | GCCTCAGTGAGCGAGCGAGCGCGCAGAGAGGGAG | |
| | TGGCCAACTCCATCACTAGGGGTTCCT | |
| ITR-48 | CCTGCAGGCAGCTGCGCGCTCGCTCGCTCACTGA | 47 |
| Left | GGCCGAAACGTCGGGCGACCTTTGGTCGCCCGGC | |
| | CTCAGTGAGCGAGCGAGCGCGCAGAGAGGGAGTG | |
| | GCCAACTCCATCACTAGGGGTTCCT | |

In one embodiment, a ceDNA vector for antibody or fusion protein production comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In some embodiment, the first ITR and the second ITR are both mod-ITRs, but have different sequences, or have different modifications, and thus are not the same modified ITRs, and have different 3D spatial configurations. Stated differently, a ceDNA vector with asymmetric ITRs comprises ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other. Exemplary asymmetric ITRs in the ceDNA vector for antibody or fusion protein production and for use to generate a ceDNA-plasmid are shown in Table 9A and 9B.

In an alternative embodiment, a ceDNA vector for antibody or fusion protein production comprises two symmetrical mod-ITRs—that is, both ITRs have the same sequence, but are reverse complements (inverted) of each other. In some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG with an addition of AACG between the G and A to result in the sequence ATCGAACGATCG (SEQ ID NO: 51). The corresponding 3′ ITR sense strand is CGATCGAT (the reverse complement of ATCGATCG) with an addition of CGTT (i.e. the reverse complement of AACG) between the T and C to result in the sequence CGATCGTTCGAT (SEQ ID NO: 49) (the reverse complement of ATCGAACGATCG) (SEQ ID NO: 51).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′ mod-ITR and the 3′ mod-ITR have the same or symmetrical three-dimensional spatial organization, they are encompassed for use herein as a modified ITR pair.

In some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG (SEQ ID NO: 51), and modified 3′ ITR as CGATCGTTCGAT (SEQ ID NO: 49) (i.e., the reverse complement of ATCGAACGATCG (SEQ ID NO: 51)), these modified ITRs would still be symmetrical if, for example, the 5′ ITR had the sequence of ATCGAACCATCG (SEQ ID NO: 50), where G in the addition is modified to C, and the substantially symmetrical 3′ ITR has the sequence of CGATCGTTCGAT (SEQ ID NO: 49), without the corresponding modification of the T in the addition to a. In some embodiments, such a modified ITR pair are substantially symmetrical as the modified ITR pair has symmetrical stereochemistry.

Table 10 shows exemplary symmetric modified ITR pairs (i.e. a left modified ITRs and the symmetric right modified ITR) for use in a ceDNA vector for antibody or fusion protein production. The bold (red) portion of the sequences identify partial ITR sequences (i.e., sequences of A-A′, C-C′ and B-B′ loops), also shown in FIGS. 31A-46B. These exemplary modified ITRs can comprise the RBE of GCGCGCTCGCTCGCTC-3′ (SEQ ID NO: 60), spacer of ACTGAGGC (SEQ ID NO: 69), the spacer complement GCCTCAGT (SEQ ID NO: 70) and RBE′ (i.e., complement to RBE) of GAGCGAGCGAGCGCGC (SEQ ID NO: 71).

TABLE 10
Exemplary symmetric modified ITR pairs in a ceDNA vector for
antibody or fusion protein production

| LEFT modified ITR | | Symmetric RIGHT modified ITR | |
|---|---|---|---|
| (modified 5′ ITR) | | (modified 3′ ITR) | |
| SEQ ID | CCTGCAGGCAGCTGCGCGCTC | SEQ ID NO: 15 | AGGAACCCCTAGTGATG |
| NO: 32 | GCTCGCTCACTGAGGCCGCC | (ITR-18, right) | GAGTTGGCCACTCCCTCT |
| (ITR-33 | CGGGAAACCCGGGCGTGCGC | | CTGCGCGCTCGCTCGC |
| left) | CTCAGTGAGCGAGCGAGCGC | | TCACTGAGGCGCACGC |
| | GCAGAGAGGGAGTGGCCAACT | | CCGGGTTTCCCGGGCG |
| | CCATCACTAGGGGTTCCT | | GCCTCAGTGAGCGAGC |
| | | | GAGCGCGCAGCTGCCT |
| | | | GCAGG |
| SEQ ID | CCTGCAGGCAGCTGCGCGCTC | SEQ ID NO: 48 | AGGAACCCCTAGTGATG |
| NO: 33 | GCTCGCTCACTGAGGCCGTC | (ITR-51, right) | GAGTTGGCCACTCCCTCT |
| (ITR-34 | GGGCGACCTTTGGTCGCCCG | | CTGCGCGCTCGCTCGC |
| left) | GCCTCAGTGAGCGAGCGAGC | | TCACTGAGGCCGGGCG |
| | GCGCAGAGAGGGAGTGGCCA | | ACCAAAGGTCGCCCGA |
| | ACTCCATCACTAGGGGTTCCT | | CGGCCTCAGTGAGCGA |
| | | | GCGAGCGCGCAGCTGC |
| | | | CTGCAGG |
| SEQ ID | CCTGCAGGCAGCTGCGCGCTC | SEQ ID NO: 16 | AGGAACCCCTAGTGATG |
| NO: 34 | GCTCGCTCACTGAGGCCGCC | (ITR-19, right) | GAGTTGGCCACTCCCTCT |
| (ITR-35 | CGGGCAAAGCCCGGGCGTCG | | CTGCGCGCTCGCTCGC |
| left) | GCCTCAGTGAGCGAGCGAGC | | TCACTGAGGCCGACGC |
| | GCGCAGAGAGGGAGTGGCCA | | CCGGGCTTTGCCCGGG |
| | ACTCCATCACTAGGGGTTCCT | | CGGCCTCAGTGAGCGA |
| | | | GCGAGCGCGCAGCTGC |
| | | | CTGCAGG |
| SEQ ID | CCTGCAGGCAGCTGCGCGCTC | SEQ ID NO: 17 | AGGAACCCCTAGTGATG |
| NO: 35 | GCTCGCTCACTGAGGCGCCC | (ITR-20, right) | GAGTTGGCCACTCCCTCT |
| (ITR-36 | GGGCGTCGGGCGACCTTTGG | | CTGCGCGCTCGCTCGC |
| left) | TCGCCCGGCCTCAGTGAGCG | | TCACTGAGGCCGGGCG |
| | AGCGAGCGCGCAGAGAGGGA | | ACCAAAGGTCGCCCGA |
| | GTGGCCAACTCCATCACTAGG | | CGCCCGGGCGCCTCAG |
| | GGTTCCT | | TGAGCGAGCGAGCGCG |
| | | | CAGCTGCCTGCAGG |
| SEQ ID | CCTGCAGGCAGCTGCGCGCTC | SEQ ID NO: 18 | AGGAACCCCTAGTGATG |
| NO: 36 | GCTCGCTCACTGAGGCAAAG | (ITR-21, right) | GAGTTGGCCACTCCCTCT |
| (ITR-37 | CCTCAGTGAGCGAGCGAGCG | | CTGCGCGCTCGCTCGC |
| left) | CGCAGAGAGGGAGTGGCCAAC | | TCACTGAGGCTTTGCC |
| | TCCATCACTAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 19 | AGGAACCCCTAGTGATG |
| NO: 37 | GCTCGCTCACTGAGGCCGCC | (ITR-22 right) | GAGTTGGCCACTCCCTCT |
| (ITR-38 | CGGGCAAAGCCCGGGCGTCG | | CTGCGCGCTCGCTCGC |
| left) | GGCGACTTTGTCGCCCGGCC | | TCACTGAGGCCGGGCG |
| | TCAGTGAGCGAGCGAGCGCG | | ACAAAGTCGCCCGACG |
| | CAGAGAGGGAGTGGCCAACTC | | CCCGGGCTTTGCCCGG |
| | CATCACTAGGGGTTCCT | | GCGGCCTCAGTGAGCG |
| | | | AGCGAGCGCGCAGCTG |
| | | | CCTGCAGG |
| SEQ ID | CCTGCAGGCAGCTGCGCGCTC | SEQ ID NO: 20 | AGGAACCCCTAGTGATG |
| NO: 38 | GCTCGCTCACTGAGGCCGCC | (ITR-23, right) | GAGTTGGCCACTCCCTCT |
| (ITR-39 | CGGGCAAAGCCCGGGCGTCG | | CTGCGCGCTCGCTCGC |
| left) | GGCGATTTTCGCCCGGCCTC | | TCACTGAGGCCGGGCG |
| | AGTGAGCGAGCGAGCGCGCA | | AAAATCGCCCGACGCC |
| | GAGAGGGAGTGGCCAACTCCA | | CGGGCTTTGCCCGGGC |
| | TCACTAGGGGTTCCT | | GGCCTCAGTGAGCGAG |
| | | | CGAGCGCGCAGCTGCC |
| | | | TGCAGG |
| SEQ ID | CCTGCAGGCAGCTGCGCGCTC | SEQ ID NO: 21 | AGGAACCCCTAGTGATG |
| NO: 39 | GCTCGCTCACTGAGGCCGCC | (ITR-24, right) | GAGTTGGCCACTCCCTCT |
| (ITR-40 | CGGGCAAAGCCCGGGCGTCG | | CTGCGCGCTCGCTCGC |
| left) | GGCGTTTCGCCCGGCCTCAG | | TCACTGAGGCCGGGCG |
| | TGAGCGAGCGAGCGCGCAGA | | AAACGCCCGACGCCCG |
| | GAGGGAGTGGCCAACTCCATC | | GGCTTTGCCCGGGCGG |
| | ACTAGGGGTTCCT | | CCTCAGTGAGCGAGCG |
| | | | AGCGCGCAGCTGCCTGC |
| | | | AGG |
| SEQ ID | CCTGCAGGCAGCTGCGCGCTC | SEQ ID NO: 22 | AGGAACCCCTAGTGATG |
| NO: 40 | GCTCGCTCACTGAGGCCGCC | (ITR-25 right) | GAGTTGGCCACTCCCTCT |
| (ITR-41 | CGGGCAAAGCCCGGGCGTCG | | CTGCGCGCTCGCTCGC |
| left) | GGCTTTGCCCGGCCTCAGTG | | TCACTGAGGCCGGGCA |
| | AGCGAGCGAGCGCGCAGAGA | | AAGCCCGACGCCCGGG |
| | GGGAGTGGCCAACTCCATCAC | | CTTTGCCCGGGCGGCC |
| | TAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 23 | AGGAACCCCTAGTGATG |
| NO: 41 | GCTCGCTCACTGAGGCCGCC | (ITR-26 right) | GAGTTGGCCACTCCCTCT |
| (ITR-42 | CGGGAAACCCGGGCGTCGGG | | CTGCGCGCTCGCTCGC |
| left) | CGACCTTTGGTCGCCCGGCC | | TCACTGAGGCCGGGCG |
| | TCAGTGAGCGAGCGAGCGCG | | ACCAAAGGTCGCCCGA |
| | CAGAGAGGGAGTGGCCAACTC | | CGCCCGGGTTTCCCGG |
| | CATCACTAGGGGTTCCT | | GCGGCCTCAGTGAGCG |
| | | | AGCGAGCGCGCAGCTG |
| | | | CCTGCAGG |
| SEQ ID | CCTGCAGGCAGCTGCGCGCTC | SEQ ID NO: 24 | AGGAACCCCTAGTGATG |
| NO: 42 | GCTCGCTCACTGAGGCCGCC | (ITR-27 right) | GAGTTGGCCACTCCCTCT |
| (ITR-43 | CGGAAACCGGGCGTCGGGCG | | CTGCGCGCTCGCTCGC |
| left) | ACCTTTGGTCGCCCGGCCTC | | TCACTGAGGCCGGGCG |
| | AGTGAGCGAGCGAGCGCGCA | | ACCAAAGGTCGCCCGA |
| | GAGAGGGAGTGGCCAACTCCA | | CGCCCGGTTTCCGGGC |
| | TCACTAGGGGTTCCT | | GGCCTCAGTGAGCGAG |
| | | | CGAGCGCGCAGCTGCC |
| | | | TGCAGG |
| SEQ ID | CCTGCAGGCAGCTGCGCGCTC | SEQ ID NO: 25 | AGGAACCCCTAGTGATG |
| NO: 43 | GCTCGCTCACTGAGGCCGCC | (ITR-28 right) | GAGTTGGCCACTCCCTCT |
| (ITR-44 | CGAAACGGGCGTCGGGCGAC | | CTGCGCGCTCGCTCGC |
| left) | CTTTGGTCGCCCGGCCTCAG | | TCACTGAGGCCGGGCG |
| | TGAGCGAGCGAGCGCGCAGA | | ACCAAAGGTCGCCCGA |
| | GAGGGAGTGGCCAACTCCATC | | CGCCCGTTTCGGGCGG |
| | ACTAGGGGTTCCT | | CCTCAGTGAGCGAGCG |
| | | | AGCGCGCAGCTGCCTGC |
| | | | AGG |
| SEQ ID | CCTGCAGGCAGCTGCGCGCTC | SEQ ID NO: 26 | AGGAACCCCTAGTGATG |
| NO: 44 | GCTCGCTCACTGAGGCCGCC | (ITR-29, right) | GAGTTGGCCACTCCCTCT |
| (ITR-45 | CAAAGGGCGTCGGGCGACCT | | CTGCGCGCTCGCTCGC |
| left) | TTGGTCGCCCGGCCTCAGTG | | TCACTGAGGCCGGGCG |
| | AGCGAGCGAGCGCGCAGAGA | | ACCAAAGGTCGCCCGA |
| | GGGAGTGGCCAACTCCATCAC | | CGCCCTTTGGGCGGCC |
| | TAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 27 | AGGAACCCCTAGTGATG |
| NO: 45 | GCTCGCTCACTGAGGCCGCC | (ITR-30, right) | GAGTTGGCCACTCCCTCT |
| (ITR-46 | AAAGGCGTCGGGCGACCTTT | | CTGCGCGCTCGCTCGC |
| left) | GGTCGCCCGGCCTCAGTGAG | | TCACTGAGGCCGGGCG |
| | CGAGCGAGCGCGCAGAGAGG | | ACCAAAGGTCGCCCGA |
| | GAGTGGCCAACTCCATCACTA | | CGCCTTTGGCGGCCTC |
| | GGGGTTCCT | | AGTGAGCGAGCGAGCG |
| | | | CGCAGCTGCCTGCAGG |
| SEQ ID | CCTGCAGGCAGCTGCGCGCTC | SEQ ID NO: 28 | AGGAACCCCTAGTGATG |
| NO: 46 | GCTCGCTCACTGAGGCCGCA | (ITR-31, right) | GAGTTGGCCACTCCCTCT |
| (ITR-47, | AAGCGTCGGGCGACCTTTGG | | CTGCGCGCTCGCTCGC |
| left) | TCGCCCGGCCTCAGTGAGCG | | TCACTGAGGCCGGGCG |
| | AGCGAGCGCGCAGAGAGGGA | | ACCAAAGGTCGCCCGA |
| | GTGGCCAACTCCATCACTAGG | | CGCTTTGCGGCCTCAG |
| | GGTTCCT | | TGAGCGAGCGAGCGCG |
| | | | CAGCTGCCTGCAGG |
| SEQ ID | CCTGCAGGCAGCTGCGCGCTC | SEQ ID NO: 29 | AGGAACCCCTAGTGATG |
| NO: 47 | GCTCGCTCACTGAGGCCGAA | (ITR-32 right) | GAGTTGGCCACTCCCTCT |
| (ITR-48, | ACGTCGGGCGACCTTTGGTC | | CTGCGCGCTCGCTCGC |
| left) | GCCCGGCCTCAGTGAGCGAG | | TCACTGAGGCCGGGCG |
| | CGAGCGCGCAGAGAGGGAGT | | ACCAAAGGTCGCCCGA |
| | GGCCAACTCCATCACTAGGGG | | CGTTTCGGCCTCAGTG |
| | TTCCT | | AGCGAGCGAGCGCGCA |
| | | | GCTGCCTGCAGG |

In some embodiments, a ceDNA vector for antibody or fusion protein production comprising an asymmetric ITR pair can comprise an ITR with a modification corresponding to any of the modifications in ITR sequences or ITR partial sequences shown in any one or more of Tables 9A-9B herein, or the sequences shown in FIG. 7A-7B of International Application PCT/US20181064242, filed Dec. 6, 2018, which is incorporated herein in its entirety, or disclosed in Tables 2, 3, 4, 5, 6, 7, 8, 9 or 10A-10B of International application PCT/US18/49996 filed Sep. 7, 2018 which is incorporated herein in its entirety by reference.

### V. Exemplary ceDNA Vectors

As described above, the present disclosure relates to recombinant ceDNA expression vectors and ceDNA vectors that encode an antibody or fusion protein for antibody or fusion protein production, comprising any one of: an asymmetrical ITR pair, a symmetrical ITR pair, or substantially symmetrical ITR pair as described above. In certain embodiments, the disclosure relates to recombinant ceDNA vectors for antibody or fusion protein production having flanking ITR sequences and a transgene, where the ITR sequences are asymmetrical, symmetrical or substantially symmetrical relative to each other as defined herein, and the ceDNA further comprises a nucleotide sequence of interest (for example an expression cassette comprising the nucleic acid of a transgene) located between the flanking ITRs, wherein said nucleic acid molecule is devoid of viral capsid protein coding sequences.

The ceDNA expression vector for antibody or fusion protein production may be any ceDNA vector that can be conveniently subjected to recombinant DNA procedures including nucleotide sequence(s) as described herein, provided at least one ITR is altered. The ceDNA vectors for antibody or fusion protein production of the present disclosure are compatible with the host cell into which the ceDNA vector is to be introduced. In certain embodiments, the ceDNA vectors may be linear. In certain embodiments, the ceDNA vectors may exist as an extrachromosomal entity. In certain embodiments, the ceDNA vectors of the present disclosure may contain an element(s) that permits integration of a donor sequence into the host cell's genome. As used herein “transgene” and “heterologous nucleotide sequence” are synonymous, and encode an antibody or fusion protein, as described herein.

Referring now to FIGS. 1A-1G, schematics of the functional components of two non-limiting plasmids useful in making a ceDNA vector for antibody or fusion protein production are shown. FIG. 1A, 1B, 1D, IF show the construct of ceDNA vectors or the corresponding sequences of ceDNA plasmids for antibody or fusion protein production. ceDNA vectors are capsid-free and can be obtained from a plasmid encoding in this order: a first ITR, an expressible transgene cassette and a second ITR, where the first and second ITR sequences are asymmetrical, symmetrical or substantially symmetrical relative to each other as defined herein. ceDNA vectors for antibody or fusion protein production are capsid-free and can be obtained from a plasmid encoding in this order: a first ITR, an expressible transgene (protein or nucleic acid) and a second ITR, where the first and second ITR sequences are asymmetrical, symmetrical or substantially symmetrical relative to each other as defined herein. In some embodiments, the expressible transgene cassette includes, as needed: an enhancer/promoter, one or more homology arms, a donor sequence, a post-transcription regulatory element (e.g., WPRE, e.g., SEQ ID NO: 67)), and a polyadenylation and termination signal (e.g., BGH polyA, e.g., SEQ ID NO: 68).

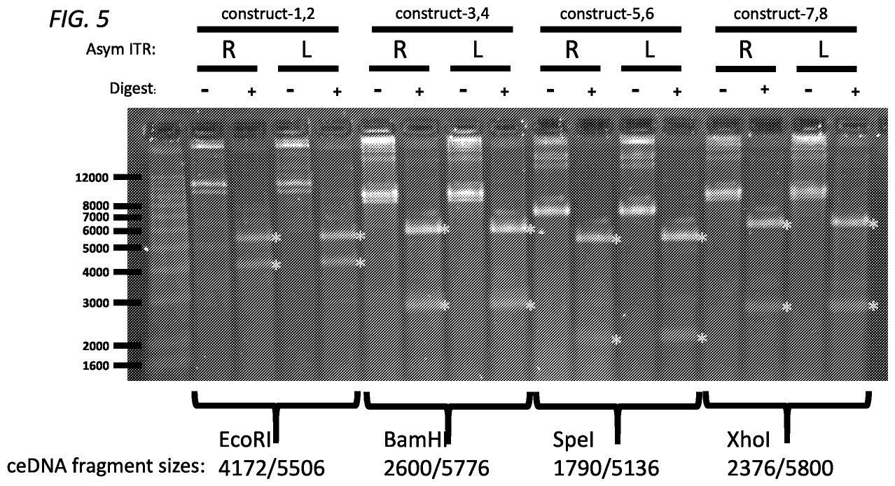

FIG. 5 is a gel confirming the production of ceDNA from multiple plasmid constructs using the method described in the Examples. The ceDNA is confirmed by a characteristic band pattern in the gel, as discussed with respect to FIG. 4A above and in the Examples.

A. Regulatory Elements.

The ceDNA vectors for antibody or fusion protein production as described herein comprising an asymmetric ITR pair or symmetric ITR pair as defined herein, can further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments, the ITR can act as the promoter for the transgene, e.g., antibody or fusion protein. In some embodiments, the ceDNA vector for antibody or fusion protein production as described herein comprises additional components to regulate expression of the transgene, for example, regulatory switches as described herein, to regulate the expression of the transgene, or a kill switch, which can kill a cell comprising the ceDNA vector encoding an antibody or antigen binding fragment thereof. Regulatory elements, including Regulatory Switches that can be used in the present invention are more fully discussed in International application PCT/US18/49996, which is incorporated herein in its entirety by reference.

In embodiments, the second nucleotide sequence includes a regulatory sequence, and a nucleotide sequence encoding a nuclease. In certain embodiments the gene regulatory sequence is operably linked to the nucleotide sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleotide sequence encoding the nuclease(s) of the present disclosure. In certain embodiments, the second nucleotide sequence includes an intron sequence linked to the 5′ terminus of the nucleotide sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

The ceDNA vectors for antibody or fusion protein production produced synthetically, or using a cell-based production method as described herein in the Examples, can further comprise a specific combination of cis-regulatory elements such as WHP posttranscriptional regulatory element (WPRE) (e.g., SEQ ID NO: 67) and BGH polyA (SEQ ID NO: 68). Suitable expression cassettes for use in expression constructs are not limited by the packaging constraint imposed by the viral capsid.

### (i). Promoters:

It will be appreciated by one of ordinary skill in the art that promoters used in the ceDNA vectors for antibody or fusion protein production as disclosed herein should be tailored as appropriate for the specific sequences they are promoting.

Expression cassettes of the ceDNA vector for antibody or fusion protein production can include a promoter, which can influence overall expression levels as well as cell-specificity. For transgene expression, e.g., antibody or antigen-binding fragment expression, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. In some embodiments, an expression cassette can contain a synthetic regulatory element, such as a CAG promoter (SEQ ID NO: 72). The CAG promoter comprises (i) the cytomegalovirus (CMV) early enhancer element, (ii) the promoter, the first exon and the first intron of chicken beta-actin gene, and (iii) the splice acceptor of the rabbit beta-globin gene. Alternatively, an expression cassette can contain an Alpha-1-antitrypsin (AAT) promoter (SEQ ID NO: 73 or SEQ ID NO: 74), a liver specific (LP1) promoter (SEQ ID NO: 75 or SEQ ID NO: 76), or a Human elongation factor-1 alpha (EF1a) promoter (e.g., SEQ ID NO: 77 or SEQ ID NO: 78). In some embodiments, the expression cassette includes one or more constitutive promoters, for example, a retroviral Rous sarcoma virus (RSV) LTR promoter (optionally with the RSV enhancer), or a cytomegalovirus (CMV) immediate early promoter (optionally with the CMV enhancer, e.g., SEQ ID NO: 79). Alternatively, an inducible promoter, a native promoter for a transgene, a tissue-specific promoter, or various promoters known in the art can be used.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 80) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 81 or SEQ ID NO: 155), a CAG promoter, a human alpha 1-antitypsin (HAAT) promoter (e.g., SEQ ID NO: 82), and the like. In certain embodiments, these promoters are altered at their downstream intron containing end to include one or more nuclease cleavage sites. In certain embodiments, the DNA containing the nuclease cleavage site(s) is foreign to the promoter DNA.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers, (e.g. SEQ ID NO: 79 and SEQ ID NO: 83), including a SV40 enhancer (SEQ ID NO: 126).

In some embodiments, a promoter may also be a promoter from a human gene such as human ubiquitin C (hUbC), human actin, human myosin, human hemoglobin, human muscle creatine, or human metallothionein. The promoter may also be a tissue specific promoter, such as a liver specific promoter, such as human alpha 1-antitypsin (HAAT), natural or synthetic. In one embodiment, delivery to the liver can be achieved using endogenous ApoE specific targeting of the composition comprising a ceDNA vector to hepatocytes via the low density lipoprotein (LDL) receptor present on the surface of the hepatocyte.

Non-limiting examples of suitable promoters for use in accordance with the present invention include the CAG promoter of, for example (SEQ ID NO: 72), the HAAT promoter (SEQ ID NO: 82), the human EF1-α promoter (SEQ ID NO: 77) or a fragment of the EF1a promoter (SEQ ID NO: 78), IE2 promoter (e.g., SEQ ID NO: 84) and the rat EF1-α promoter (SEQ ID NO: 85), mEF1 promoter (SEQ ID NO: 59), or IE1 promoter fragment (SEQ ID NO: 125).

### (ii). Polyadenylation Sequences:

A sequence encoding a polyadenylation sequence can be included in the ceDNA vector for antibody or fusion protein production to stabilize an mRNA expressed from the ceDNA vector, and to aid in nuclear export and translation. In one embodiment, the ceDNA vector does not include a polyadenylation sequence. In other embodiments, the ceDNA vector for antibody or fusion protein production includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between. As shown in FIGS. 10A-10G, a polyadenylation sequence can be located 3′ of the transgene encoding an antibody or antibody fragment. In some embodiments, a ceDNA vector for antibody or fusion protein production which encodes a full IgG or full antibody can comprise an IRES (internal ribosome entry site) sequence (SEQ ID NO: 190), e.g., where the IRES sequence is located 3′ of a polyadenylation sequence, such that a second transgene (e.g., antibody or antigen-binding fragment) that is located 3′ of a first transgene, is translated and expressed by the same ceDNA vector, such that the ceDNA vector can express a full antibody (see, e.g., FIG. 10B).

The expression cassettes can include a poly-adenylation sequence known in the art or a variation thereof, such as a naturally occurring sequence isolated from bovine BGHpA (e.g., SEQ ID NO: 68) or a virus SV40pA (e.g., SEQ ID NO: 86), or a synthetic sequence (e.g., SEQ ID NO: 87). Some expression cassettes can also include SV40 late polyA signal upstream enhancer (USE) sequence. In some embodiments, the, USE can be used in combination with SV40pA or heterologous poly-A signal.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 67) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 88 and SEQ ID NO: 89.

(iii). Nuclear Localization Sequences

In some embodiments, the ceDNA vector for antibody or fusion protein production comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 11.

TABLE 11
Nuclear Localization Signals

| | | SEQ |
|---|---|---|
| | | ID |
| SOURCE | SEQUENCE | NO. |
| SV40 | PKKKRKV | 90 |
| virus | (encoded by | |
| large | CCCAAGAAGAAGAGGAAGGTG; | |
| T- | SEQ ID NO: 91) | |
| antigen | | |
| nucleoplasmin | KRPAATKKAGQAKKKK | 92 |
| c-myc | PAAKRVKLD | 93 |
| | RQRRNELKRSP | 94 |
| hRNPA1 | NQSSNFGPMKGGNFGGRSSGPYGGGG | 95 |
| M9 | QYFAKPRNQGGY | |
| IBB | RMRIZFKNKGKDTAELRRRRVEVSVE | 96 |
| domain | LRKAKKDEQILKRRNV | |
| from | | |
| importin- | | |
| alpha | | |
| myoma T | VSRKRPRP | 97 |
| protein | PPKKARED | 98 |
| human p53 | PQPKKKPL | 99 |
| mouse | SALIKKKKKMAP | 100 |
| c-abl IV | | |
| influenza | DRLRR | 117 |
| virus | | |
| NS1 | PKQKKRK | 118 |
| Hepatitis | RKLKKKIKKL | 119 |
| virus | | |
| delta | | |
| antigen | | |
| mouse Mx1 | REKKKFLKRR | 120 |
| protein | | |
| human | KRKGDEVDGVDEVAKKKSKK | 121 |
| poly | | |
| (ADP- | | |
| ribose) | | |
| polymerase | | |
| steroid | RKCLQAGMNLEARKTKK | 122 |
| hormone | | |
| receptors | | |
| (human) | | |
| glucocorticoid | | |

B. Additional Components of ceDNA Vectors

The ceDNA vectors for antibody or fusion protein production of the present disclosure may contain nucleotides that encode other components for gene expression. For example, to select for specific gene targeting events, a protective shRNA may be embedded in a microRNA and inserted into a recombinant ceDNA vector designed to integrate site-specifically into the highly active locus, such as an albumin locus. Such embodiments may provide a system for in vivo selection and expansion of gene-modified hepatocytes in any genetic background such as described in Nygaard et al., A universal system to select gene-modified hepatocytes in vivo, Gene Therapy, Jun. 8, 2016. The ceDNA vectors of the present disclosure may contain one or more selectable markers that permit selection of transformed, transfected, transduced, or the like cells. A selectable marker is a gene the product of which provides for biocide or viral resistance, resistance to heavy metals, prototrophy to auxotrophs, NeoR, and the like. In certain embodiments, positive selection markers are incorporated into the donor sequences such as NeoR. Negative selections markers may be incorporated downstream the donor sequences, for example a nucleic acid sequence HSV-tk encoding a negative selection marker may be incorporated into a nucleic acid construct downstream the donor sequence.

### C. Regulatory Switches

A molecular regulatory switch is one which generates a measurable change in state in response to a signal. Such regulatory switches can be usefully combined with the ceDNA vectors for antibody or fusion protein production as described herein to control the output of expression of the antibody or antigen-binding fragment from the ceDNA vector. In some embodiments, the ceDNA vector for antibody or fusion protein production comprises a regulatory switch that serves to fine tune expression of the antibody or antigen-binding fragment. For example, it can serve as a biocontainment function of the ceDNA vector. In some embodiments, the switch is an “ON/OFF” switch that is designed to start or stop (i.e., shut down) expression of the antibody or antigen-binding fragment in the ceDNA vector in a controllable and regulatable fashion. In some embodiments, the switch can include a “kill switch” that can instruct the cell comprising the ceDNA vector to undergo cell programmed death once the switch is activated. Exemplary regulatory switches encompassed for use in a ceDNA vector for antibody or fusion protein production can be used to regulate the expression of a transgene, and are more fully discussed in International application PCT/US18/49996, which is incorporated herein in its entirety by reference

### (i) Binary Regulatory Switches

In some embodiments, the ceDNA vector for antibody or fusion protein production comprises a regulatory switch that can serve to controllably modulate expression of the antibody or antigen-binding fragment. For example, the expression cassette located between the ITRs of the ceDNA vector may additionally comprise a regulatory region, e.g., a promoter, cis-element, repressor, enhancer etc., that is operatively linked to the antibody or antigen-binding fragment, where the regulatory region is regulated by one or more cofactors or exogenous agents. By way of example only, regulatory regions can be modulated by small molecule switches or inducible or repressible promoters. Non-limiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

### (ii) Small Molecule Regulatory Switches

A variety of art-known small-molecule based regulatory switches are known in the art and can be combined with the ceDNA vectors for antibody or fusion protein production as disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3^{rd}; Nat Methods. 2013, 10(11):1085-8. In some embodiments, the regulatory switch to control the transgene or expressed by the ceDNA vector is a pro-drug activation switch, such as that disclosed in U.S. Pat. Nos. 8,771,679, and 6,339,070.

(iii) “Passcode” Regulatory Switches

In some embodiments the regulatory switch can be a “passcode switch” or “passcode circuit”. Passcode switches allow fine tuning of the control of the expression of the transgene from the ceDNA vector when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “Passcode circuit” encompassed for use in the ceDNA vector comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis. In some embodiments, a regulatory switch for use in a passcode system can be selected from any or a combination of the switches disclosed in Table 11 of International Patent Application PCT/US18/49996, which is incorporated herein in its entirety by reference.

### (iv). Nucleic Acid-Based Regulatory Switches to Control Transgene Expression

In some embodiments, the regulatory switch to control the antibody or antigen-binding fragment expressed by the ceDNA is based on a nucleic-acid based control mechanism. Exemplary nucleic acid control mechanisms are known in the art and are envisioned for use. For example, such mechanisms include riboswitches, such as those disclosed in, e.g., US2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6(3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the ceDNA vector can comprise a regulatory switch that encodes a RNAi molecule that is complementary to the to part of the transgene expressed by the ceDNA vector. When such RNAi is expressed even if the transgene (e.g., antibody or antigen-binding fragment) is expressed by the ceDNA vector, it will be silenced by the complementary RNAi molecule, and when the RNAi is not expressed when the transgene is expressed by the ceDNA vector the transgene (e.g., antibody or antigen-binding fragment) is not silenced by the RNAi.

In some embodiments, the regulatory switch is a tissue-specific self-inactivating regulatory switch, for example as disclosed in US2002/0022018, whereby the regulatory switch deliberately switches transgene (e.g., antibody or antigen-binding fragment) expression off at a site where transgene expression might otherwise be disadvantageous. In some embodiments, the regulatory switch is a recombinase reversible gene expression system, for example as disclosed in US2014/0127162 and U.S. Pat. No. 8,324,436.

### (v). Post-Transcriptional and Post-Translational Regulatory Switches.

In some embodiments, the regulatory switch to control the antibody or antigen-binding fragment expressed by the ceDNA vector is a post-transcriptional modification system. For example, such a regulatory switch can be an aptazyme riboswitch that is sensitive to tetracycline or theophylline, as disclosed in US2018/0119156, GB201107768, WO2001/064956A3, EP Patent 2707487 and Beilstein et al., ACS Synth. Biol., 2015, 4 (5), pp 526-534; Zhong et al., Elife. 2016 Nov. 2; 5. pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

### (vi). Other Exemplary Regulatory Switches

Any known regulatory switch can be used in the ceDNA vector to control the gene expression of the antibody or antigen-binding fragment expressed by the ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the ceDNA vector.

In some embodiments, a regulatory switch envisioned for use in the ceDNA vector is a hypoxia-mediated or stress-activated switch, e.g., such as those disclosed in WO1999060142A2, U.S. Pat. Nos. 5,834,306; 6,218,179; 6,709,858; US2015/0322410; Greco et al., (2004) Targeted Cancer Therapies 9, S368, as well as FROG, TOAD and NRSE elements and conditionally inducible silence elements, including hypoxia response elements (HREs), inflammatory response elements (IREs) and shear-stress activated elements (SSAEs), e.g., as disclosed in U.S. Pat. No. 9,394,526. Such an embodiment is useful for turning on expression of the transgene from the ceDNA vector after ischemia or in ischemic tissues, and/or tumors.

### (iv). Kill Switches

Other embodiments described herein relate to a ceDNA vector for antibody or fusion protein production as described herein comprising a kill switch. A kill switch as disclosed herein enables a cell comprising the ceDNA vector to be killed or undergo programmed cell death as a means to permanently remove an introduced ceDNA vector from the subject's system. It will be appreciated by one of ordinary skill in the art that use of kill switches in the ceDNA vectors for antibody or fusion protein production would be typically coupled with targeting of the ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the ceDNA vector in the absence of an input survival signal or other specified condition. Stated another way, a kill switch encoded by a ceDNA vector for antibody or fusion protein production as described herein can restrict cell survival of a cell comprising a ceDNA vector to an environment defined by specific input signals. Such kill switches serve as a biological biocontainment function should it be desirable to remove the ceDNA vector expressing the antibody or antigen-binding fragment from a subject or to ensure that it will not express the encoded antibody or antigen-binding fragment.

Other kill switches known to a person of ordinary skill in the art are encompassed for use in the ceDNA vector for antibody or fusion protein production as disclosed herein, e.g., as disclosed in US2010/0175141; US2013/0009799; US2011/0172826; US2013/0109568, as well as kill switches disclosed in Jusiak et al, Reviews in Cell Biology and molecular Medicine; 2014; 1-56; Kobayashi et al., PNAS, 2004; 101; 8419-9; Marchisio et al., Int. Journal of Biochem and Cell Biol., 2011; 43; 310-319; and in Reinshagen et al., Science Translational Medicine, 2018, 11.

Accordingly, in some embodiments, the ceDNA vector for antibody or fusion protein production can comprise a kill switch nucleic acid construct, which comprises the nucleic acid encoding an effector toxin or reporter protein, where the expression of the effector toxin (e.g., a death protein) or reporter protein is controlled by a predetermined condition. For example, a predetermined condition can be the presence of an environmental agent, such as, e.g., an exogenous agent, without which the cell will default to expression of the effector toxin (e.g., a death protein) and be killed. In alternative embodiments, a predetermined condition is the presence of two or more environmental agents, e.g., the cell will only survive when two or more necessary exogenous agents are supplied, and without either of which, the cell comprising the ceDNA vector is killed.

In some embodiments, the ceDNA vector for antibody or fusion protein production is modified to incorporate a kill-switch to destroy the cells comprising the ceDNA vector to effectively terminate the in vivo expression of the transgene being expressed by the ceDNA vector (e.g., full length antibody, Fab, scAb). Specifically, the ceDNA vector is further genetically engineered to express a switch-protein that is not functional in mammalian cells under normal physiological conditions. Only upon administration of a drug or environmental condition that specifically targets this switch-protein, the cells expressing the switch-protein will be destroyed thereby terminating the expression of the therapeutic protein or peptide. For instance, it was reported that cells expressing HSV-thymidine kinase can be killed upon administration of drugs, such as ganciclovir and cytosine deaminase. See, for example, Dey and Evans, Suicide Gene Therapy by Herpes Simplex Virus-1 Thymidine Kinase (HSV-TK), in Targets in Gene Therapy, edited by You (2011); and Beltinger et al., Proc. Natl. Acad. Sci. USA 96(15):8699-8704 (1999). In some embodiments the ceDNA vector can comprise a siRNA kill switch referred to as DISE (Death Induced by Survival gene Elimination) (Murmann et al., Oncotarget. 2017; 8:84643-84658. Induction of DISE in ovarian cancer cells in vivo).

### VI. Detailed Method of Production of a ceDNA Vector

A. Production in General

Certain methods for the production of a ceDNA vector for antibody or fusion protein production comprising an asymmetrical ITR pair or symmetrical ITR pair as defined herein is described in section IV of International application PCT/US18/49996 filed Sep. 7, 2018, which is incorporated herein in its entirety by reference. In some embodiments, a ceDNA vector for antibody or fusion protein production as disclosed herein can be produced using insect cells, as described herein. In alternative embodiments, a ceDNA vector for antibody or fusion protein production as disclosed herein can be produced synthetically and in some embodiments, in a cell-free method, as disclosed on International Application PCT/US19/14122, filed Jan. 18, 2019, which is incorporated herein in its entirety by reference.

As described herein, in one embodiment, a ceDNA vector for antibody or fusion protein production can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the invention provides for use of host cell lines that have stably integrated the DNA vector polynucleotide expression template (ceDNA template) into their own genome in production of the non-viral DNA vector, e.g. as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors for antibody or fusion protein production as described herein are insect cells, and baculovirus is used to deliver both the polynucleotide that encodes Rep protein and the non-viral DNA vector polynucleotide expression construct template for ceDNA, e.g., as described in FIGS. 4A-4C and Example 1. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

The presence of the ceDNA vector for antibody or fusion protein production can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA. FIG. 4C and FIG. 4D illustrate one embodiment for identifying the presence of the closed ended ceDNA vectors produced by the processes herein.

B. ceDNA Plasmid

A ceDNA-plasmid is a plasmid used for later production of a ceDNA vector for antibody or fusion protein production. In some embodiments, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one aspect, a ceDNA vector for antibody or fusion protein production is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In a further embodiment, the ceDNA-plasmid system is devoid of viral capsid protein coding sequences (i.e. it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present invention can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at www web address: oesys.springer.de/viruses/database/mkchapter.asp?virID=42.04.)(note—references to a URL or database refer to the contents of the URL or database as of the effective filing date of this application) In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An exemplary ceDNA (e.g., rAAV0) vector for antibody or fusion protein production is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

C. Exemplary Method of Making the ceDNA Vectors from ceDNA Plasmids

Methods for making capsid-less ceDNA vectors for antibody or fusion protein production are also provided herein, notably a method with a sufficiently high yield to provide sufficient vector for in vivo experiments.

In some embodiments, a method for the production of a ceDNA vector for antibody or fusion protein production comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

D. Cell Lines:

Host cell lines used in the production of a ceDNA vector for antibody or fusion protein production can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, Hep1A, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

CeDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

E. Isolating and Purifying ceDNA Vectors:

Examples of the process for obtaining and isolating ceDNA vectors are described in FIGS. 4A-4E and the specific examples below. ceDNA-vectors for antibody or fusion protein production disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids that encode an antibody heavy chain and/or an antibody light chain, or plasmids encoding one or more REP proteins. An exemplary ceDNA plasmid is shown in FIG. 6A, where the transgene encoding aducanumab HC and the transgene encoding aducanuman LC can be replaced with nucleic acid sequences with the heavy chain and/or light chain of an antibody or fusion protein of interest, e.g. see Tables 1-5.

In one aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA vector for antibody or fusion protein production are described herein. Expression constructs used for generating a ceDNA vector for antibody or fusion protein production as described herein can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors for antibody or fusion protein production can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors for antibody or fusion protein production as described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one non-limiting example, the process can be performed by loading the supernatant on an ion exchange column (e.g. SARTOBIND Q®) which retains nucleic acids, and then eluting (e.g. with a 1.2 M NaCl solution) and performing a further chromatographic purification on a gel filtration column (e.g. 6 fast flow GE). The capsid-free AAV vector is then recovered by, e.g., precipitation.

In some embodiments, ceDNA vectors for antibody or fusion protein production can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the invention herein relates to methods of purifying ceDNA vectors from host cell lines that have stably integrated a ceDNA construct into their own genome. In one embodiment, ceDNA vectors are purified as DNA molecules. In another embodiment, the ceDNA vectors are purified as exosomes or microparticles.

FIG. 5 of International application PCT/US18/49996 shows a gel confirming the production of ceDNA from multiple ceDNA-plasmid constructs using the method described in the Examples. The ceDNA is confirmed by a characteristic band pattern in the gel, as discussed with respect to FIG. 4D in the Examples.

### VII. Pharmaceutical Compositions

In another aspect, pharmaceutical compositions are provided. The pharmaceutical composition comprises a ceDNA vector for antibody or fusion protein production as described herein and a pharmaceutically acceptable carrier or diluent.

The ceDNA vectors for antibody or fusion protein production as disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier. For example, the ceDNA vectors for antibody or fusion protein production as described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization including a ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a ceDNA vector for antibody or fusion protein production can be formulated to deliver a transgene for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

A ceDNA vector for antibody or fusion protein production as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more ceDNA vectors for antibody or fusion protein production as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA for antibody or fusion protein production can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA for antibody or fusion protein production to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids, such as ceDNA vectors for antibody or fusion protein production can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

ceDNA vectors for antibody or fusion protein production as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector for antibody or fusion protein production as disclosed herein can be delivered into hematopoietic stem cells, for example, by the methods as described, for example, in U.S. Pat. No. 5,928,638.

The ceDNA vectors for antibody or fusion protein production in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations, including but not limited to polyethylene glycol (PEG)-functional group containing compounds are disclosed in International Application PCT/US2018/050042, filed on Sep. 7, 2018 and in International application PCT/US2018/064242, filed on Dec. 6, 2018, e.g., see the section entitled “Pharmaceutical Formulations”.

Various delivery methods known in the art or modification thereof can be used to deliver ceDNA vectors in vitro or in vivo. For example, in some embodiments, ceDNA vectors for antibody or fusion protein production are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. In some cases, a ceDNA vector alone is directly injected as naked DNA into skin, thymus, cardiac muscle, skeletal muscle, or liver cells. In some cases, a ceDNA vector is delivered by gene gun. Gold or tungsten spherical particles (1-3 m diameter) coated with capsid-free AAV vectors can be accelerated to high speed by pressurized gas to penetrate into target tissue cells.

Compositions comprising a ceDNA vector for antibody or fusion protein production and a pharmaceutically acceptable carrier are specifically contemplated herein. In some embodiments, the ceDNA vector is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods, or ultrasound.

In some cases, a ceDNA vector for antibody or fusion protein production is delivered by hydrodynamic injection, which is a simple and highly efficient method for direct intracellular delivery of any water-soluble compounds and particles into internal organs and skeletal muscle in an entire limb.

In some cases, ceDNA vectors for antibody or fusion protein production are delivered by ultrasound by making nanoscopic pores in membrane to facilitate intracellular delivery of DNA particles into cells of internal organs or tumors, so the size and concentration of plasmid DNA have great role in efficiency of the system. In some cases, ceDNA vectors are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### A. Exosomes:

In some embodiments, a ceDNA vector for antibody or fusion protein production as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

### B. Microparticle/Nanoparticles:

In some embodiments, a ceDNA vector for antibody or fusion protein production as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector for antibody or fusion protein production as disclosed herein. For example, various delivery methods using lipid nanoparticles are described in U.S. Pat. Nos. 9,404,127, 9,006,417 and 9,518,272.

In some embodiments, a ceDNA vector for antibody or fusion protein production as disclosed herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334.

### C. Conjugates

In some embodiments, a ceDNA vector for antibody or fusion protein production as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a ceDNA vector for antibody or fusion protein production as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a ceDNA vector for antibody or fusion protein production as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a ceDNA vector for antibody or fusion protein production as disclosed herein is conjugated to a carbohydrate, for example as described in U.S. Pat. No. 8,450,467.

### D. Nanocapsule

Alternatively, nanocapsule formulations of a ceDNA vector for antibody or fusion protein production as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### E. Liposomes

The ceDNA vectors for antibody or fusion protein production in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

### F. Exemplary Liposome and Lipid Nanoparticle (LNP) Compositions

The ceDNA vectors for antibody or fusion protein production in accordance with the present invention can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA vectors are disclosed in International Application PCT/US2018/050042, filed on Sep. 7, 2018, and International Application PCT/US2018/064242, filed on Dec. 6, 2018 which are incorporated herein in their entirety and envisioned for use in the methods and compositions for ceDNA vectors for antibody or fusion protein production as disclosed herein.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In some aspects, the disclosure provides for a lipid nanoparticle comprising ceDNA and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

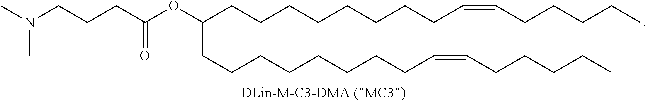 [SMILES: CCCCC/C=C\CCCCCCCCC(CCCCCCCC/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, content of which is incorporated herein by reference in its entirety.

In some embodiments, the ionizable lipid is (13Z,16Z)-N,N-dimethyl-3-nonyldocosa-13,16-dien-1-amine (Compound 32), as described in WO2012/040184, content of which is incorporated herein by reference in its entirety.

In some embodiments, the ionizable lipid is Compound 6 or Compound 22 as described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions as disclosed herein are described in International Application PCT/US2018/050042, filed on Sep. 7, 2018, and PCT/US2018/064242, filed on Dec. 6, 2018 which is incorporated herein in its entirety. Exemplary non-cationic lipids are described in International Application Publication WO2017/099823 and US patent publication US2018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids. In some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International application WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In some embodiments, a PEG-lipid is a compound as defined in US2018/0028664, the content of which is incorporated herein by reference in its entirety. In some embodiments, a PEG-lipid is disclosed in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000],

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. 5,885,613, 6,287,591, 6,320,017, and 6,586,559, the contents of all of which are incorporated herein by reference in their entirety.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the ceDNA within the LNP is useful for treating cancer, the additional compound can be an anti-cancer agent (e.g., a chemotherapeutic agent, a targeted cancer therapy (including, but not limited to, a small molecule or an antibody). In another example, if the LNP containing the ceDNA is useful for treating an infection, the additional compound can be an antimicrobial agent (e.g., an antibiotic or antiviral compound). In yet another example, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a ceDNA encoding a different protein or a different compound, such as a therapeutic may be used in the compositions and methods of the invention.

In some embodiments, the additional compound is an immune modulating agent. For example, the additional compound is an immunosuppressant. In some embodiments, the additional compound is immune stimulatory agent. Also provided herein is a pharmaceutical composition comprising the lipid nanoparticle-encapsulated insect-cell produced, or a synthetically produced ceDNA vector for antibody or fusion protein production as described herein and a pharmaceutically acceptable carrier or excipient.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

The ceDNA vector can be complexed with the lipid portion of the particle or encapsulated in the lipid position of the lipid nanoparticle. In some embodiments, the ceDNA can be fully encapsulated in the lipid position of the lipid nanoparticle, thereby protecting it from degradation by a nuclease, e.g., in an aqueous solution. In some embodiments, the ceDNA in the lipid nanoparticle is not substantially degraded after exposure of the lipid nanoparticle to a nuclease at 37° C. for at least about 20, 30, 45, or 60 minutes. In some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense. In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

### VIII. Methods of Use

A ceDNA vector for antibody or fusion protein production as disclosed herein can also be used in a method for the delivery of a nucleotide sequence of interest (e.g., encoding an antibody or fusion protein) to a target cell (e.g., a host cell). The method may in particular be a method for delivering an antibody or antigen-binding fragment to a cell of a subject in need thereof and treating a disease of interest. The invention allows for the in vivo expression of an antibody or fusion protein. encoded in the ceDNA vector in a cell in a subject such that therapeutic effect of the expression of the antibody or fusion protein occurs. These results are seen with both in vivo and in vitro modes of ceDNA vector delivery.

In addition, the invention provides a method for the delivery of an antibody or fusion protein in a cell of a subject in need thereof, comprising multiple administrations of the ceDNA vector of the invention encoding said antibody or fusion protein. Since the ceDNA vector of the invention does not induce an immune response like that typically observed against encapsidated viral vectors, such a multiple administration strategy will likely have greater success in a ceDNA-based system.

The ceDNA vector are administered in sufficient amounts to transfect the cells of a desired tissue and to provide sufficient levels of gene transfer and expression of the antibody or fusion protein without undue adverse effects. Conventional and pharmaceutically acceptable routes of administration include, but are not limited to, intravenous (e.g., in a liposome formulation), direct delivery to the selected organ (e.g., intraportal delivery to the liver), intramuscular, and other parental routes of administration. Routes of administration may be combined, if desired.

Delivery of a ceDNA vector for antibody or fusion protein production as described herein is not limited to delivery of the expressed antibody or antigen-binding fragment. For example, conventionally produced (e.g., using a cell-based production method (e.g., insect-cell production methods) or synthetically produced ceDNA vectors as described herein may be used with other delivery systems provided to provide a portion of the gene therapy. One non-limiting example of a system that may be combined with the ceDNA vectors in accordance with the present disclosure includes systems which separately deliver one or more co-factors or immune suppressors for effective gene expression of the ceDNA vector expressing the antibody or fusion protein.

The invention also provides for a method of treating a disease in a subject comprising introducing into a target cell in need thereof (in particular a muscle cell or tissue) of the subject a therapeutically effective amount of a ceDNA vector, optionally with a pharmaceutically acceptable carrier. While the ceDNA vector can be introduced in the presence of a carrier, such a carrier is not required. The ceDNA vector selected comprises a nucleotide sequence encoding an antibody or fusion protein useful for treating the disease. In particular, the ceDNA vector may comprise a desired antibody or fusion protein sequence operably linked to control elements capable of directing transcription of the desired antibody or fusion protein encoded by the exogenous DNA sequence when introduced into the subject. The ceDNA vector can be administered via any suitable route as provided above, and elsewhere herein.

The compositions and vectors provided herein can be used to deliver an antibody or fusion protein for various purposes. In some embodiments, the transgene encodes an antibody or fusion protein that is intended to be used for research purposes, e.g., to create a somatic transgenic animal model harboring the transgene, e.g., to study the function of the antibody or fusion protein product. In another example, the transgene encodes an antibody or fusion protein that is intended to be used to create an animal model of disease. In some embodiments, the encoded antibody or fusion protein is useful for the treatment or prevention of disease states in a mammalian subject. The antibody or fusion protein can be transferred (e.g., expressed in) to a patient in a sufficient amount to treat a disease associated with reduced expression, lack of expression or dysfunction of the gene.

In principle, the expression cassette can include a nucleic acid or any transgene that encodes an antibody or fusion protein that is either reduced or absent due to a mutation or which conveys a therapeutic benefit when overexpressed is considered to be within the scope of the invention. Preferably, noninserted bacterial DNA is not present and preferably no bacterial DNA is present in the ceDNA compositions provided herein.

A ceDNA vector is not limited to one species of ceDNA vector. As such, in another aspect, multiple ceDNA vectors expressing different antibodies or fusion proteins or the same antibody or fusion protein but operatively linked to different promoters or cis-regulatory elements can be delivered simultaneously or sequentially to the target cell, tissue, organ, or subject. Therefore, this strategy can allow for the gene therapy or gene delivery of multiple antibodies and/or fusion proteins simultaneously. It is also possible to separate different portions of the antibody into separate ceDNA vectors (e.g., different domains and/or co-factors required for functionality of the antibody or antigen-binding fragment) which can be administered simultaneously or at different times, and can be separately regulatable, thereby adding an additional level of control of expression of the antibody or fusion protein. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The invention also provides for a method of treating a disease in a subject comprising introducing into a target cell in need thereof (in particular a muscle cell or tissue) of the subject a therapeutically effective amount of a ceDNA vector as disclosed herein, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

### IX. Methods of Delivering ceDNA Vectors for Antibody or Fusion Protein Production

In some embodiments, a ceDNA vector for antibody or fusion protein production can be delivered to a target cell in vitro or in vivo by various suitable methods. ceDNA vectors alone can be applied or injected. CeDNA vectors can be delivered to a cell without the help of a transfection reagent or other physical means. Alternatively, ceDNA vectors for antibody or fusion protein production can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

The ceDNA vectors for antibody or fusion protein production as disclosed herein can efficiently target cell and tissue-types that are normally difficult to transduce with conventional AAV virions using various delivery reagent.

One aspect of the technology described herein relates to a method of delivering an antibody or antigen-binding fragment to a cell. Typically, for in vivo and in vitro methods, a ceDNA vector for antibody or fusion protein production as disclosed herein may be introduced into the cell using the methods as disclosed herein, as well as other methods known in the art. A ceDNA vector for antibody or fusion protein production as disclosed herein are preferably administered to the cell in a biologically-effective amount. If the ceDNA vector is administered to a cell in vivo (e.g., to a subject), a biologically-effective amount of the ceDNA vector is an amount that is sufficient to result in transduction and expression of the antibody or antigen-binding fragment in a target cell.

Exemplary modes of administration of a ceDNA vector for antibody or fusion protein production as disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., but not limited to, liver, eye, muscles, including skeletal muscle, cardiac muscle, diaphragm muscle, or brain).

Administration of the ceDNA vector can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the ceDNA vector can also be to a tumor (e.g., in or near a tumor or a lymph node).

The most suitable route in any given case will depend on the nature and severity of the condition being treated, ameliorated, and/or prevented and on the nature of the particular ceDNA vector that is being used. Additionally, ceDNA permits one to administer more than one antibody in a single vector, or multiple ceDNA vectors (e.g. a ceDNA cocktail).

A. Intramuscular Administration of a ceDNA Vector

In some embodiments, a method of treating a disease in a subject comprises introducing into a target cell in need thereof (in particular a muscle cell or tissue) of the subject a therapeutically effective amount of a ceDNA vector encoding an antibody or antigen binding fragment thereof, optionally with a pharmaceutically acceptable carrier. In some embodiments, the ceDNA vector for antibody or fusion protein production is administered to a muscle tissue of a subject.

In some embodiments, administration of the ceDNA vector can be to any site in a subject, including, without limitation, a site selected from the group consisting of a skeletal muscle, a smooth muscle, the heart, the diaphragm, or muscles of the eye. In some embodiments, a ceDNA vector according to the present invention is administered to skeletal muscle, diaphragm muscle and/or cardiac muscle (e.g., to treat, ameliorate and/or prevent muscular dystrophy or heart disease (e.g., PAD or congestive heart failure).

Administration of a ceDNA vector for antibody or fusion protein production as disclosed herein to a skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA as disclosed herein vector can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In embodiments, the ceDNA vector as disclosed herein can be administered without employing “hydrodynamic” techniques. For instance, tissue delivery (e.g., to muscle) of conventional viral vectors is often enhanced by hydrodynamic techniques (e.g., intravenous/intravenous administration in a large volume), which increase pressure in the vasculature and facilitate the ability of the viral vector to cross the endothelial cell barrier. In particular embodiments, the ceDNA vectors described herein can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Furthermore, a composition comprising a ceDNA vector for antibody or fusion protein production as disclosed herein that is administered to a skeletal muscle can be administered to a skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

Administration of a ceDNA vector for antibody or fusion protein production as disclosed herein to diaphragm muscle can be by any suitable method including intravenous administration, intra-arterial administration, and/or intra-peritoneal administration. In some embodiments, delivery of an expressed transgene from the ceDNA vector to a target tissue can also be achieved by delivering a synthetic depot comprising the ceDNA vector, where a depot comprising the ceDNA vector is implanted into skeletal, smooth, cardiac and/or diaphragm muscle tissue or the muscle tissue can be contacted with a film or other matrix comprising the ceDNA vector as described herein. Such implantable matrices or substrates are described in U.S. Pat. No. 7,201,898.

Administration of a ceDNA vector for antibody or fusion protein production as disclosed herein to cardiac muscle includes administration to the left atrium, right atrium, left ventricle, right ventricle and/or septum. The ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration of a ceDNA vector for antibody or fusion protein production as disclosed herein to smooth muscle can be by any suitable method including intravenous administration, intra-arterial administration, and/or intra-peritoneal administration. In one embodiment, administration can be to endothelial cells present in, near, and/or on smooth muscle. Non-limiting examples of smooth muscles include the iris of the eye, bronchioles of the lung, laryngeal muscles (vocal cords), muscular layers of the stomach, esophagus, small and large intestine of the gastrointestinal tract, ureter, detrusor muscle of the urinary bladder, uterine myometrium, penis, or prostate gland.

In some embodiments, of a ceDNA vector for antibody or fusion protein production as disclosed herein is administered to skeletal muscle, diaphragm muscle and/or cardiac muscle (e.g., to treat, ameliorate and/or prevent muscular dystrophy or heart disease (e.g., PAD or congestive heart failure). In representative embodiments, a ceDNA vector according to the present invention is used to treat and/or prevent disorders of skeletal, cardiac and/or diaphragm muscle.

Specifically, it is contemplated that a composition comprising a ceDNA vector for antibody or fusion protein production as disclosed herein can be delivered to one or more muscles of the eye (e.g., Lateral rectus, Medial rectus, Superior rectus, Inferior rectus, Superior oblique, Inferior oblique), facial muscles (e.g., Occipitofrontalis muscle, Temporoparietalis muscle, Procerus muscle, Nasalis muscle, Depressor septi nasi muscle, Orbicularis oculi muscle, Corrugator supercilii muscle, Depressor supercilii muscle, Auricular muscles, Orbicularis oris muscle, Depressor anguli oris muscle, Risorius, Zygomaticus major muscle, Zygomaticus minor muscle, Levator labii superioris, Levator labii superioris alaeque nasi muscle, Depressor labii inferioris muscle, Levator anguli oris, Buccinator muscle, Mentalis) or tongue muscles (e.g., genioglossus, hyoglossus, chondroglossus, styloglossus, palatoglossus, superior longitudinal muscle, inferior longitudinal muscle, the vertical muscle, and the transverse muscle).

(i) Intramuscular injection: In some embodiments, a composition comprising a ceDNA vector for antibody or fusion protein production as disclosed herein can be injected into one or more sites of a given muscle, for example, skeletal muscle (e.g., deltoid, vastus lateralis, ventrogluteal muscle of dorsogluteal muscle, or anterolateral thigh for infants) in a subject using a needle. The composition comprising ceDNA can be introduced to other subtypes of muscle cells. Non-limiting examples of muscle cell subtypes include skeletal muscle cells, cardiac muscle cells, smooth muscle cells and/or diaphragm muscle cells.

Methods for intramuscular injection are known to those of skill in the art and as such are not described in detail herein. However, when performing an intramuscular injection, an appropriate needle size should be determined based on the age and size of the patient, the viscosity of the composition, as well as the site of injection. Table 12 provides guidelines for exemplary sites of injection and corresponding needle size:

TABLE 12
Guidelines for intramuscular injection in human patients

| | | | Maximum |
|---|---|---|---|
| | | | volume of |
| Injection Site | Needle Gauge | Needle Size | composition |
| Ventrogluteal site | Aqueous | Thin adult: 15 to 25 mm | 3 mL |
| (gluteus medius | solutions: 20-25 | Average adult: 25 mm | |
| and gluteus | gauge | Larger adult (over 150 lbs): 25 to | |
| minimus) | Viscous or oil- | 38 mm. | |
| | based solution: | Children and infants: will require | |
| | 18-21 gauge | a smaller needle | |
| Vastus lateralis | Aqueous | Adult: 25 mm to 38 mm | 3 mL |
| | solutions: 20-25 | | |
| | gauge | | |
| | Viscous or oil- | | |
| | based solution: | | |
| | 18-21 gauge | | |
| | Children/infants: | | |
| | 22 to 25 gauge | | |
| Deltoid | 22 to 25 gauge | Males: | 1 mL |
| | | 130-260 lbs: 25 mm | |
| | | Females: | |
| | | <130 lbs: 16 mm | |
| | | 130-200 lbs: 25 mm | |
| | | >200 lbs: 38 mm | |

In certain embodiments, a ceDNA vector for antibody or fusion protein production as disclosed herein is formulated in a small volume, for example, an exemplary volume as outlined in Table 12 for a given subject. In some embodiments, the subject can be administered a general or local anesthetic prior to the injection, if desired. This is particularly desirable if multiple injections are required or if a deeper muscle is injected, rather than the common injection sites noted above.

In some embodiments, intramuscular injection can be combined with electroporation, delivery pressure or the use of transfection reagents to enhance cellular uptake of the ceDNA vector.

(H) Transfection Reagents: In some embodiments, a ceDNA vector for antibody or fusion protein production as disclosed herein is formulated in compositions comprising one or more transfection reagents to facilitate uptake of the vectors into myotubes or muscle tissue. Thus, in one embodiment, the nucleic acids described herein are administered to a muscle cell, myotube or muscle tissue by transfection using methods described elsewhere herein.

(iii) Electroporation: In certain embodiments, a ceDNA vector for antibody or fusion protein production as disclosed herein is administered in the absence of a carrier to facilitate entry of ceDNA into the cells, or in a physiologically inert pharmaceutically acceptable carrier (i.e., any carrier that does not improve or enhance uptake of the capsid free, non-viral vectors into the myotubes). In such embodiments, the uptake of the capsid free, non-viral vector can be facilitated by electroporation of the cell or tissue.

Cell membranes naturally resist the passage of extracellular into the cell cytoplasm. One method for temporarily reducing this resistance is “electroporation”, where electrical fields are used to create pores in cells without causing permanent damage to the cells. These pores are large enough to allow DNA vectors, pharmaceutical drugs, DNA, and other polar compounds to gain access to the interior of the cell. With time, the pores in the cell membrane close and the cell once again becomes impermeable.

Electroporation can be used in both in vitro and in vivo applications to introduce e.g., exogenous DNA into living cells. In vitro applications typically mix a sample of live cells with the composition comprising e.g., DNA. The cells are then placed between electrodes such as parallel plates and an electrical field is applied to the cell/composition mixture.

There are a number of methods for in vivo electroporation; electrodes can be provided in various configurations such as, for example, a caliper that grips the epidermis overlying a region of cells to be treated. Alternatively, needle-shaped electrodes may be inserted into the tissue, to access more deeply located cells. In either case, after the composition comprising e.g., nucleic acids are injected into the treatment region, the electrodes apply an electrical field to the region. In some electroporation applications, this electric field comprises a single square wave pulse on the order of 100 to 500 V/cm. of about 10 to 60 ms duration. Such a pulse may be generated, for example, in known applications of the Electro Square Porator T820, made by the BTX Division of Genetronics, Inc.

Typically, successful uptake of e.g., nucleic acids occurs only if the muscle is electrically stimulated immediately, or shortly after administration of the composition, for example, by injection into the muscle.

In certain embodiments, electroporation is achieved using pulses of electric fields or using low voltage/long pulse treatment regimens (e.g., using a square wave pulse electroporation system). Exemplary pulse generators capable of generating a pulsed electric field include, for example, the ECM600, which can generate an exponential wave form, and the ElectroSquarePorator (T820), which can generate a square wave form, both of which are available from BTX, a division of Genetronics, Inc. (San Diego, Calif.). Square wave electroporation systems deliver controlled electric pulses that rise quickly to a set voltage, stay at that level for a set length of time (pulse length), and then quickly drop to zero.

In some embodiments, a local anesthetic is administered, for example, by injection at the site of treatment to reduce pain that may be associated with electroporation of the tissue in the presence of a composition comprising a capsid free, non-viral vector as described herein. In addition, one of skill in the art will appreciate that a dose of the composition should be chosen that minimizes and/or prevents excessive tissue damage resulting in fibrosis, necrosis or inflammation of the muscle.

(iv) Delivery Pressure: In some embodiments, delivery of a ceDNA vector for antibody or fusion protein production as disclosed herein to muscle tissue is facilitated by delivery pressure, which uses a combination of large volumes and rapid injection into an artery supplying a limb (e.g., iliac artery). This mode of administration can be achieved through a variety of methods that involve infusing limb vasculature with a composition comprising a ceDNA vector, typically while the muscle is isolated from the systemic circulation using a tourniquet of vessel clamps. In one method, the composition is circulated through the limb vasculature to permit extravasation into the cells. In another method, the intravascular hydrodynamic pressure is increased to expand vascular beds and increase uptake of the ceDNA vector into the muscle cells or tissue. In one embodiment, the ceDNA composition is administered into an artery.

(v) Lipid Nanoparticle Compositions: In some embodiments, a ceDNA vector for antibody or fusion protein production as disclosed herein for intramuscular delivery are formulated in a composition comprising a liposome as described elsewhere herein.

(vi) Systemic Administration of a ceDNA Vector targeted to Muscle Tissue: In some embodiments, a ceDNA vector for antibody or fusion protein production as disclosed herein is formulated to be targeted to the muscle via indirect delivery administration, where the ceDNA is transported to the muscle as opposed to the liver. Accordingly, the technology described herein encompasses indirect administration of compositions comprising a ceDNA vector for antibody or fusion protein production as disclosed herein to muscle tissue, for example, by systemic administration. Such compositions can be administered topically, intravenously (by bolus or continuous infusion), intracellular injection, intratissue injection, orally, by inhalation, intraperitoneally, subcutaneously, intracavity, and can be delivered by peristaltic means, if desired, or by other means known by those skilled in the art. The agent can be administered systemically, for example, by intravenous infusion, if so desired.

In some embodiments, uptake of a ceDNA vector for antibody or fusion protein production as disclosed herein into muscle cells/tissue is increased by using a targeting agent or moiety that preferentially directs the vector to muscle tissue. Thus, in some embodiments, a capsid free, ceDNA vector can be concentrated in muscle tissue as compared to the amount of capsid free ceDNA vectors present in other cells or tissues of the body.

In some embodiments, the composition comprising a ceDNA vector for antibody or fusion protein production as disclosed herein further comprises a targeting moiety to muscle cells. In other embodiments, the expressed gene product comprises a targeting moiety specific to the tissue in which it is desired to act. The targeting moiety can include any molecule, or complex of molecules, which is/are capable of targeting, interacting with, coupling with, and/or binding to an intracellular, cell surface, or extracellular biomarker of a cell or tissue. The biomarker can include, for example, a cellular protease, a kinase, a protein, a cell surface receptor, a lipid, and/or fatty acid. Other examples of biomarkers that the targeting moieties can target, interact with, couple with, and/or bind to include molecules associated with a particular disease. For example, the biomarkers can include cell surface receptors implicated in cancer development, such as epidermal growth factor receptor and transferrin receptor. The targeting moieties can include, but are not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds) that bind to molecules expressed in the target muscle tissue.

In certain embodiments, the targeting moiety may further comprise a receptor molecule, including, for example, receptors, which naturally recognize a specific desired molecule of a target cell. Such receptor molecules include receptors that have been modified to increase their specificity of interaction with a target molecule, receptors that have been modified to interact with a desired target molecule not naturally recognized by the receptor, and fragments of such receptors (see, e.g., Skerra, 2000, J. Molecular Recognition, 13:167-187). A preferred receptor is a chemokine receptor. Exemplary chemokine receptors have been described in, for example, Lapidot et al, 2002, Exp Hematol, 30:973-81 and Onuffer et al, 2002, Trends Pharmacol Sci, 23:459-67.

In other embodiments, the additional targeting moiety may comprise a ligand molecule, including, for example, ligands which naturally recognize a specific desired receptor of a target cell, such as a Transferrin (Tf) ligand. Such ligand molecules include ligands that have been modified to increase their specificity of interaction with a target receptor, ligands that have been modified to interact with a desired receptor not naturally recognized by the ligand, and fragments of such ligands.

In still other embodiments, the targeting moiety may comprise an aptamer. Aptamers are oligonucleotides that are selected to bind specifically to a desired molecular structure of the target cell. Aptamers typically are the products of an affinity selection process similar to the affinity selection of phage display (also known as in vitro molecular evolution). The process involves performing several tandem iterations of affinity separation, e.g., using a solid support to which the diseased immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In some embodiments, the targeting moiety can comprise a photo-degradable ligand (i.e., a ‘caged’ ligand) that is released, for example, from a focused beam of light such that the capsid free, non-viral vectors or the gene product are targeted to a specific tissue.

It is also contemplated herein that the compositions be delivered to multiple sites in one or more muscles of the subject. That is, injections can be in at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 injections sites. Such sites can be spread over the area of a single muscle or can be distributed among multiple muscles.

B. Administration of the ceDNA Vector for Antibody or Fusion Protein Production to Non-Muscle Locations

In another embodiment, a ceDNA vector for antibody or fusion protein production is administered to the CNS (e.g., to the brain or to the eye). The ceDNA vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vector may also be administered to different regions of the eye such as the retina, cornea and/or optic nerve. The ceDNA vector may be delivered into the cerebrospinal fluid (e.g., by lumbar puncture). The ceDNA vector for antibody or fusion protein production may further be administered intravascularly to the CNS in situations in which the blood-brain barrier has been perturbed (e.g., brain tumor or cerebral infarct).

In some embodiments, the ceDNA vector for antibody or fusion protein production can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the ceDNA vector for antibody or fusion protein production is administered in a liquid formulation by direct injection (e.g., stereotactic injection) to the desired region or compartment in the CNS. In other embodiments, the ceDNA vector can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898). In yet additional embodiments, the ceDNA vector can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vector can be delivered to muscle tissue from which it can migrate into neurons.

### C. Ex Vivo Treatment

In some embodiments, cells are removed from a subject, a ceDNA vector for antibody or fusion protein production as disclosed herein is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector for antibody or fusion protein production as disclosed herein are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, a ceDNA vector for antibody or fusion protein production as disclosed herein can encode an antibody or fusion protein as described herein (sometimes called a transgene or heterologous nucleotide sequence) that is to be produced in a cell in vitro, ex vivo, or in vivo. For example, in contrast to the use of the ceDNA vectors described herein in a method of treatment as discussed herein, in some embodiments a ceDNA vector for antibody or fusion protein production may be introduced into cultured cells and the expressed antibody or fusion protein isolated from the cells, e.g., for the production of antibodies and fusion proteins. In some embodiments, the cultured cells comprising a ceDNA vector for antibody or fusion protein production as disclosed herein can be used for commercial production of antibodies or fusion proteins, e.g., serving as a cell source for small or large scale biomanufacturing of antibodies or fusion proteins. In alternative embodiments, a ceDNA vector for antibody or fusion protein production as disclosed herein is introduced into cells in a host non-human subject, for in vivo production of antibodies or fusion proteins, including small scale production as well as for commercial large scale antibody or fusion protein production.

The ceDNA vectors for antibody or fusion protein production as disclosed herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

### D. Dose Ranges

Provided herein are methods of treatment comprising administering to the subject an effective amount of a composition comprising a ceDNA vector encoding an antibody or fusion protein as described herein. As will be appreciated by a skilled practitioner, the term “effective amount” refers to the amount of the ceDNA composition administered that results in expression of the antibody or fusion protein in a “therapeutically effective amount” for the treatment of a disease.

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems.

A ceDNA vectors for antibody or fusion protein production as disclosed herein is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vectors for antibody or fusion protein production as disclosed herein required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1 μg to 100 g of the ceDNA vector. If exosomes or microparticles are used to deliver the ceDNA vector, then a therapeutically effective dose can be determined experimentally, but is expected to deliver from 1 μg to about 100 g of vector. Moreover, a therapeutically effective dose is an amount ceDNA vector that expresses a sufficient amount of the transgene to have an effect on the subject that results in a reduction in one or more symptoms of the disease, but does not result in significant off-target or significant adverse side effects. In one embodiment, a “therapeutically effective amount” is an amount of an expressed antibody or fusion protein that is sufficient to produce a statistically significant, measurable change in expression of a disease biomarker or reduction of a given disease symptom. Such effective amounts can be gauged in clinical trials as well as animal studies for a given ceDNA vector composition.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of a ceDNA vectors for antibody or fusion protein production as disclosed herein to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

For the treatment of a disease, the appropriate dosage of a ceDNA vector that expresses an antibody or fusion protein as disclosed herein will depend on the specific type of disease to be treated, the type of antibody, the severity and course of the disease, previous therapy, the patient's clinical history and response to the antibody, and the discretion of the attending physician. The antibody is suitably administered to the patient at one time or over a series of treatments. Various dosing schedules including, but not limited to, single or multiple administrations over various time-points, bolus administration, and pulse infusion are contemplated herein.

Depending on the type and severity of the disease, a ceDNA vector is administered in an amount that the encoded antibody or fusion protein is expressed at about 0.3 mg/kg to 100 mg/kg (e.g. 15 mg/kg-100 mg/kg, or any dosage within that range), by one or more separate administrations, or by continuous infusion. One typical daily dosage of the ceDNA vector is sufficient to result in the expression of the encoded antibody or fusion protein at a range from about 15 mg/kg to 100 mg/kg or more, depending on the factors mentioned above. One exemplary dose of the ceDNA vector is an amount sufficient to result in the expression of the encoded antibody or fusion protein as disclosed herein in a range from from about 10 mg/kg to about 50 mg/kg. Thus, one or more doses of a ceDNA vector in an amount sufficient to result in the expression of the encoded antibody or fusion protein at about 0.5 mg/kg, 1 mg/kg, 1.5 mg/kg, 2.0 mg/kg, 3 mg/kg, 4.0 mg/kg, 5 mg/kg, 10 mg/kg, 15 mg/kg, 20 mg/kg, 25 mg/kg, 30 mg/kg, 35 mg/kg, 40 mg/kg, 50 mg/kg, 60 mg/kg, 70 mg/kg, 80 mg/kg, 90 mg/kg, or 100 mg/kg (or any combination thereof) may be administered to the patient. In some embodiments, the ceDNA vector is an amount sufficient to result in the expression of the encoded antibody or fusion protein for a total dose in the range of 50 mg to 2500 mg. An exemplary dose of a ceDNA vector is an amount sufficient to result in the total expression of the encoded antibody or fusion protein at about 50 mg, about 100 mg, 200 ng, 300 mg, 400 mg, about 500 mg, about 600 mg, about 700 mg, about 720 mg, about 1000 mg, about 1050 mg, about 1100 mg, about 1200 mg, about 1300 mg, about 1400 mg, about 1500 mg, about 1600 mg, about 1700 mg, about 1800 mg, about 1900 mg, about 2000 mg, about 2050 mg, about 2100 mg, about 2200 mg, about 2300 mg, about 2400 mg, or about 2500 mg (or any combination thereof). As the expression of the antibody or fusion protein from ceDNA vector can be carefully controlled by regulatory switches herein, or alternatively multiple dose of the ceDNA vector administered to the subject, the expression of the antibody or fusion protein from the ceDNA vector can be controlled in such a way that the doses of the expressed antibody or fusion protein may be administered intermittently. e.g. every week, every two weeks, every three weeks, every four weeks, every month, every two months, every three months, or every six months from the ceDNA vector. The progress of this therapy can be monitored by conventional techniques and assays.

In certain embodiments, a ceDNA vector is administered an amount sufficient to result in the expression of the encoded antibody or fusion protein at a dose of 15 mg/kg, 30 mg/kg, 40 mg/kg, 45 mg/kg, 50 mg/kg, 60 mg/kg or a flat dose, e.g., 300 mg, 500 mg, 700 mg, 800 mg, or higher. In some embodiments, the expression of the antibody or fusion protein from the ceDNA vector is controlled such that the antibody or fusion protein is expressed every day, every other day, every week, every 2 weeks or every 4 weeks for a period of time. In some embodiments, the expression of the antibody or fusion protein from the ceDNA vector is controlled such that the antibody or fusion protein is expressed every 2 weeks or every 4 weeks for a period of time. In certain embodiments, the period of time is 6 months, one year, eighteen months, two years, five years, ten years, 15 years, 20 years, or the lifetime of the patient.

Treatment can involve administration of a single dose or multiple doses. In some embodiments, more than one dose can be administered to a subject; in fact, multiple doses can be administered as needed, because the ceDNA vector elicits does not elicit an anti-capsid host immune response due to the absence of a viral capsid. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a ceDNA vector as described by the disclosure (i.e., the absence of capsid components) allows the ceDNA vector for antibody or fusion protein production to be administered to a host on multiple occasions. In some embodiments, the number of occasions in which a heterologous nucleic acid is delivered to a subject is in a range of 2 to 10 times (e.g., 2, 3, 4, 5, 6, 7, 8, 9, or 10 times). In some embodiments, a ceDNA vector is delivered to a subject more than 10 times.

In some embodiments, a dose of a ceDNA vector for antibody or fusion protein production as disclosed herein is administered to a subject no more than once per calendar day (e.g., a 24-hour period). In some embodiments, a dose of a ceDNA vector is administered to a subject no more than once per 2, 3, 4, 5, 6, or 7 calendar days. In some embodiments, a dose of a ceDNA vector for antibody or fusion protein production as disclosed herein is administered to a subject no more than once per calendar week (e.g., 7 calendar days). In some embodiments, a dose of a ceDNA vector is administered to a subject no more than bi-weekly (e.g., once in a two calendar week period). In some embodiments, a dose of a ceDNA vector is administered to a subject no more than once per calendar month (e.g., once in 30 calendar days). In some embodiments, a dose of a ceDNA vector is administered to a subject no more than once per six calendar months. In some embodiments, a dose of a ceDNA vector is administered to a subject no more than once per calendar year (e.g., 365 days or 366 days in a leap year).

In particular embodiments, more than one administration (e.g., two, three, four or more administrations) of a ceDNA vector for antibody or fusion protein production as disclosed herein may be employed to achieve the desired level of gene expression over a period of various intervals, e.g., daily, weekly, monthly, yearly, etc.

In some embodiments, a therapeutic antibody encoded by a ceDNA vector as disclosed herein can be regulated by a regulatory switch, inducible or repressible promotor so that it is expressed in a subject for at least 1 hour, at least 2 hours, at least 5 hours, at least 10 hours, at least 12 hours, at least 18 hours, at least 24 hours, at least 36 hours, at least 48 hours, at least 72 hours, at least 1 week, at least 2 weeks, at least 1 month, at least 2 months, at least 6 months, at least 12 months/one year, at least 2 years, at least 5 years, at least 10 years, at least 15 years, at least 20 years, at least 30 years, at least 40 years, at least 50 years or more. In one embodiment, the expression can be achieved by repeated administration of the ceDNA vectors described herein at predetermined or desired intervals. Alternatively, a ceDNA vector for antibody or fusion protein production as disclosed herein can further comprise components of a gene editing system (e.g., CRISPR/Cas, TALENs, zinc finger endonucleases etc) to permit insertion of the one or more nucleic acid sequences encoding the antibody for substantially permanent treatment or “curing” the disease. Such ceDNA vectors comprising gene editing components are disclosed in International Application PCT/US18/64242, and can include the 5′ and 3′ homology arms (e.g., SEQ ID NO: 151-154, or sequences with at least 40%, 50%, 60%, 70% or 80% homology thereto) for insertion of the nucleic acid enoding the antibody into safe harbor regions, such as, but not including albumin gene or CCR5 gene.

The duration of treatment depends upon the subject's clinical progress and responsiveness to therapy. Continuous, relatively low maintenance doses are contemplated after an initial higher therapeutic dose.

### E. Unit Dosage Forms

In some embodiments, the pharmaceutical compositions comprising a ceDNA vector for antibody or fusion protein production as disclosed herein can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### X. Methods of Treatment

The technology described herein also demonstrates methods for making, as well as methods of using the disclosed ceDNA vectors for antibody or fusion protein production in a variety of ways, including, for example, ex vivo, ex situ, in vitro and in vivo applications, methodologies, diagnostic procedures, and/or gene therapy regimens.

In one embodiment, the expressed therapeutic antibody expressed from a ceDNA vector as disclosed herein is functional for the treatment of disease. In a preferred embodiment, the therapeutic antibody does not cause an immune system reaction, unless so desired.

Provided herein is a method of treating a disease or disorder in a subject comprising introducing into a target cell in need thereof (for example, a muscle cell or tissue, or other affected cell type) of the subject a therapeutically effective amount of a ceDNA vector for antibody or fusion protein production as disclosed herein, optionally with a pharmaceutically acceptable carrier. While the ceDNA vector can be introduced in the presence of a carrier, such a carrier is not required. The ceDNA vector implemented comprises a nucleotide sequence encoding an antibody or antigen-binding fragment as described herein useful for treating the disease. In particular, a ceDNA vector for antibody or fusion protein production as disclosed herein may comprise a desired antibody or antigen-binding fragment DNA sequence operably linked to control elements capable of directing transcription of the desired antibody or antigen-binding fragment encoded by the exogenous DNA sequence when introduced into the subject. The ceDNA vector for antibody or fusion protein production as disclosed herein can be administered via any suitable route as provided above, and elsewhere herein.

Disclosed herein are ceDNA vector compositions and formulations for antibody or fusion protein production as disclosed herein that include one or more of the ceDNA vectors of the present invention together with one or more pharmaceutically-acceptable buffers, diluents, or excipients. Such compositions may be included in one or more diagnostic or therapeutic kits, for diagnosing, preventing, treating or ameliorating one or more symptoms of a disease, injury, disorder, trauma or dysfunction. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a ceDNA vector for antibody or fusion protein production as disclosed herein, the method comprising providing to a cell, tissue or organ of a subject in need thereof, an amount of the ceDNA vector as disclosed herein; and for a time effective to enable expression of the antibody or antigen-binding fragment from the ceDNA vector thereby providing the subject with a diagnostically- or a therapeutically-effective amount of the antibody or antigen-binding fragment expressed by the ceDNA vector. In a further aspect, the subject is human.

Another aspect of the technology described herein provides a method for diagnosing, preventing, treating, or ameliorating at least one or more symptoms of a disease, a disorder, a dysfunction, an injury, an abnormal condition, or trauma in a subject. In an overall and general sense, the method includes at least the step of administering to a subject in need thereof one or more of the disclosed ceDNA vector for antibody or fusion protein production, in an amount and for a time sufficient to diagnose, prevent, treat or ameliorate the one or more symptoms of the disease, disorder, dysfunction, injury, abnormal condition, or trauma in the subject. In such an embodiment, the subject can be evaluated for efficacy of the antibody/antigen-binding fragment, or alternatively, detection of the antigen or antigen-binding fragment to a particular protein or tissue location (including cellular and subcellular location) in the subject. As such, the ceDNA vector for antibody or fusion protein production as disclosed herein can be used as an in vivo diagnostic tool, e.g., for the detection of cancer or other indications. In a further aspect, the subject is human.

Another aspect is use of a ceDNA vector for antibody or fusion protein production as disclosed herein as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, a ceDNA vector for antibody or fusion protein production as disclosed herein can be used to deliver antibodies or fusion proteins that neutralize a protein in a pathway that results in increasing the expression of a normal gene for replacement therapy, as well, in some embodiments, to create animal models for the diseases using ceDNA vectors expressing neutralizing antibodies or fusion proteins. For unbalanced disease states, a ceDNA vector for antibody or fusion protein production as disclosed herein can be used to create a disease state in a model system, which could then be used in efforts to counteract the disease state. Thus the ceDNA vector for antibody or fusion protein production as disclosed herein permit the treatment of genetic diseases. As used herein, a disease state is treated by partially or wholly remedying the deficiency or imbalance that causes the disease or makes it more severe.

In one embodiment, it is contemplated herein that intramuscular delivery and expression of a transgene for the antibody in a muscle can be used to treat muscle-specific diseases or, alternatively, to act as a depot for protein production for a therapeutic transgene product to act at a distant site. The ceDNA vectors described herein can be used to express an antibody or fusion protein in a muscle. In some embodiments, the gene product increases the expression and/or activity of a target gene. In other embodiments, the gene product decreases the expression and/or activity of a target gene.

### A. Host Cells:

In some embodiments, a ceDNA vector for antibody or fusion protein production as disclosed herein delivers the antibody or antigen-binding fragment transgene into a subject host cell. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including a ceDNA vector for antibody or fusion protein production as disclosed herein. Thus, one can use multiple host cells depending on the purpose as is obvious to the skilled artisan. A construct or a ceDNA vector for antibody or fusion protein production as disclosed herein including donor sequence is introduced into a host cell so that the donor sequence is maintained as a chromosomal integrant as described earlier. The term host cell encompasses any progeny of a parent cell that is not identical to the parent cell due to mutations that occur during replication. The choice of a host cell will to a large extent depend upon the donor sequence and its source.

The host cell may also be a eukaryote, such as a mammalian, insect, plant, or fungal cell. In one embodiment, the host cell is a human cell (e.g., a primary cell, a stem cell, or an immortalized cell line). In some embodiments, the host cell can be administered a ceDNA vector for antibody or fusion protein production as disclosed herein ex vivo and then delivered to the subject after the gene therapy event. A host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. In some embodiments, gene modified host cells, e.g., bone marrow stem cells, e.g., CD34⁺ cells, or induced pluripotent stem cells can be transplanted back into a patient for expression of a therapeutic protein.

### C. Additional Diseases for Gene Therapy:

In general, a ceDNA vector for antibody or fusion protein production as disclosed herein can be used to deliver any antibody or antigen-binding fragment in accordance with the description above to treat, prevent, or ameliorate the symptoms associated with any disorder related to an aborant protein expression or gene expression in a subject. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the ceDNA vectors as disclosed herein can be advantageously used in the treatment of individuals with metabolic disorders (e.g., ornithine transcarbamylase deficiency).

In some embodiments, a ceDNA vector for antibody or fusion protein production as disclosed herein can be used to treat, ameliorate, and/or prevent a disease or disorder caused by mutation in a gene or gene product. Exemplary diseases or disorders that can be treated with a ceDNA vectors include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

In some embodiments, a ceDNA vector for antibody or fusion protein production as disclosed herein can be used to deliver an antibody or fusion protein to skeletal, cardiac or diaphragm muscle, for production of an antibody or fusion protein for secretion and circulation in the blood or for systemic delivery to other tissues to treat, ameliorate, and/or prevent a disorder (e.g., a metabolic disorder, such as diabetes (e.g., insulin), hemophilia (e.g., VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid.alpha.-glucosidase] or Fabry disease [.alpha.-galactosidase A]) or a glycogen storage disorder (such as Pompe disease [lysosomal acid a glucosidase]). Other suitable proteins for treating, ameliorating, and/or preventing metabolic disorders are described above.

In other embodiments, a ceDNA vector for antibody or fusion protein production as disclosed herein can be used to deliver an antibody or antigen-binding fragment in a method of treating, ameliorating, and/or preventing a metabolic disorder in a subject in need thereof. Illustrative metabolic disorders and an antibody or antigen-binding fragment are described herein. Optionally, the polypeptide is secreted (e.g., a polypeptide that is a secreted polypeptide in its native state or that has been engineered to be secreted, for example, by operable association with a secretory signal sequence as is known in the art).

The a ceDNA vector for antibody or fusion protein production as disclosed herein can be administered to the lungs of a subject by any suitable means, optionally by administering an aerosol suspension of respirable particles comprising the ceDNA vectors, which the subject inhales. The respirable particles can be liquid or solid. Aerosols of liquid particles comprising the ceDNA vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the ceDNA vectors may likewise be produced with any solid particulate medicament aerosol generator, by techniques known in the pharmaceutical art.

In some embodiments, a ceDNA vector for antibody or fusion protein production as disclosed herein can be administered to tissues of the CNS (e.g., brain, eye). In particular embodiments, the ceDNA vectors as disclosed herein may be administered to treat, ameliorate, or prevent diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Ocular disorders that may be treated, ameliorated, or prevented with a ceDNA vector for antibody or fusion protein production as disclosed herein include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, the ceDNA vector as disclosed herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic antibodies or fusion proteins either intraocularly (e.g., in the vitreous) or periocularly (e.g., in the sub-Tenon's region). Additional ocular diseases that may be treated, ameliorated, or prevented with the ceDNA vectors of the invention include geographic atrophy, vascular or “wet” macular degeneration, Stargardt disease, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by a ceDNA vector for antibody or fusion protein production as disclosed herein. One or more anti-inflammatory antibodies or fusion proteins can be expressed by intraocular (e.g., vitreous or anterior chamber) administration of the ceDNA vector as disclosed herein.

In some embodiments, a ceDNA vector for antibody or fusion protein production as disclosed herein can encode an antibody or antigen-binding fragment that is associated with transgene encoding a reporter polypeptide (e.g., an enzyme such as Green Fluorescent Protein, or alkaline phosphatase). In some embodiments, a transgene that encodes a reporter protein useful for experimental or diagnostic purposes, is selected from any of: β-lactamase, β-galactosidase (LacZ), alkaline phosphatase, thymidine kinase, green fluorescent protein (GFP), chloramphenicol acetyltransferase (CAT), luciferase, and others well known in the art. In some aspects, ceDNA vectors expressing an antibody or antigen-binding fragment linked to a reporter polypeptide may be used for diagnostic purposes, as well as to determine efficacy or as markers of the ceDNA vector's activity in the subject to which they are administered.

In some embodiments, a ceDNA vector for antibody or fusion protein production as disclosed herein can express an antibody or antigen-binding fragment that specifically binds to an immunogenic polypeptide or immunogen in a subject. The antibody or antigen-binding fragment can specifically bind to any immunogen of interest known in the art including, but not limited to, immunogens from human immunodeficiency virus, influenza virus, gag proteins, tumor antigens, cancer antigens, bacterial antigens, viral antigens, and the like.

D. Testing for Successful Gene Expression Using a ceDNA Vector

Assays well known in the art can be used to test the efficiency of gene delivery of an antibody or antigen-binding fragment by a ceDNA vector can be performed in both in vitro and in vivo models. Levels of the expression of the antibody or antigen-binding fragment by ceDNA can be assessed by one skilled in the art by measuring mRNA and protein levels of the antibody or antigen-binding fragment (e.g., reverse transcription PCR, western blot analysis, and enzyme-linked immunosorbent assay (ELISA)). In one embodiment, ceDNA comprises a reporter protein that can be used to assess the expression of an antibody or antigen-binding fragment, for example by examining the expression of the reporter protein by fluorescence microscopy or a luminescence plate reader. For in vivo applications, protein function assays can be used to test the functionality of a given antibody or antigen-binding fragment to determine if gene expression has successfully occurred. One skilled will be able to determine the best test for measuring functionality of an antibody or antigen-binding fragment expressed by the ceDNA vector in vitro or in vivo.

It is contemplated herein that the effects of gene expression of an antibody or antigen-binding fragment from the ceDNA vector in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, an antibody or antigen-binding fragment in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

E. Determining Efficacy by Assessing Antibody Expression from the ceDNA Vector

Essentially any method known in the art for determining protein expression can be used to analyze expression of a desired antibody from a ceDNA vector. Non-limiting examples of such methods/assays include enzyme-linked immunoassay (ELISA), affinity ELISA, ELISPOT, serial dilution, flow cytometry, surface plasmon resonance analysis, kinetic exclusion assay, mass spectrometry, Western blot, immunoprecipitation, and PCR.

For assessing antibody expression in vivo, a biological sample can be obtained from a subject for analysis. Exemplary biological samples include a biofluid sample, a body fluid sample, blood (including whole blood), serum, plasma, urine, saliva, a biopsy and/or tissue sample etc. A biological sample or tissue sample can also refer to a sample of tissue or fluid isolated from an individual including, but not limited to, tumor biopsy, stool, spinal fluid, pleural fluid, nipple aspirates, lymph fluid, the external sections of the skin, respiratory, intestinal, and genitourinary tracts, tears, saliva, breast milk, cells (including, but not limited to, blood cells), tumors, organs, and also samples of in vitro cell culture constituent. The term also includes a mixture of the above-mentioned samples. The term “sample” also includes untreated or pretreated (or pre-processed) biological samples. In some embodiments, the sample used for the assays and methods described herein comprises a serum sample collected from a subject to be tested.

### F. Determining Efficacy of the Expressed Antibody by Clinical Parameters

The efficacy of a given antibody or antigen-binding fragment expressed by a ceDNA vector for a given disease (i.e., functional expression), such as rheumatoid arthritis or cancer (including, but not limited to, breast cancer, melanoma etc.) can be determined by the skilled clinician. However, a treatment is considered “effective treatment,” as the term is used herein, if any one or all of the signs or symptoms of the cancer is/are altered in a beneficial manner, or other clinically accepted symptoms or markers of disease are improved, or ameliorated, e.g., by at least 10% following treatment with a ceDNA vector encoding a therapeutic antibody as described herein. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease (e.g., arthritis, cancer); or (2) relieving the disease, e.g., causing regression of symptoms; and (3) preventing or reducing the likelihood of the development of the disease, or preventing secondary diseases/disorders associated with the disease (e.g., hand deformity from rheumatoid arthritis, or cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators that are particular to a given disease. For example, physical indicators for cancer include, but are not limited to, pain, tumor size, tumor growth rate, blood cell count, etc.

### XI. Various Applications of ceDNA Vectors Expressing Antibodies or Fusion Proteins

As disclosed herein, the compositions and ceDNA vectors for antibody or fusion protein production as described herein can be used to express an antibody or fusion protein for a range of purposes. In one embodiment, the ceDNA vector expressing an antibody or fusion protein can be used to create a somatic transgenic animal model harboring the transgene, e.g., to study the function of the antibody's or fusion protein's target. In some embodiments, a ceDNA vector expressing an antibody or fusion protein is useful for the treatment, prevention, or amelioration of disease states or disorders in a mammalian subject.

In some embodiments the antibody or fusion protein can be expressed from the ceDNA vector in a subject in a sufficient amount to treat a disease associated with increased expression, increased activity of the gene product, or inappropriate upregulation of a gene. In such an embodiment, the expressed antibody or fusion protein can be a blocking or neutralizing antibody or fusion protein that functions to inhibit or suppress or otherwise decrease the activity of the protein or gene product that is the target, or to which the antibody or fusion protein specifically binds to.

In some embodiments the antibody or fusion protein can be expressed from the ceDNA vector in a subject in a sufficient amount to treat a disease associated with a reduced expression, lack of expression or dysfunction of a protein. For example, the expressed antibody or fusion protein is an activating antibody or fusion protein, and can increase the activity or function of a protein with decreased expression and/or activity in the subject, for example, by agonizing the protein or inhibiting a repressor of the protein.

It will be appreciated by one of ordinary skill in the art that the transgene may not be an open reading frame of a gene to be transcribed itself; instead it may be a promoter region or repressor region of a target gene, and the ceDNA vector may modify such region with the outcome of so modulating the expression of a gene of interest.

The compositions and ceDNA vectors for antibody or fusion protein production as disclosed herein can be used to deliver an antibody or antigen-binding fragment for various purposes as described above.

In some embodiments, the transgene encodes one or more antibodies or fusion proteins, which are useful for the treatment, amelioration, or prevention of disease states in a mammalian subject. The antibody or fusion protein expressed by the ceDNA vector is administered to a patient in a sufficient amount to treat a disease associated with an abnormal gene sequence, which can result in any one or more of the following: increased protein expression, over activity of the protein, reduced expression, lack of expression or dysfunction of the target gene or protein.

In some embodiments, the ceDNA vectors for antibody or fusion protein production as disclosed herein are envisioned for use in diagnostic and screening methods, whereby an antibody or antigen-binding fragment is transiently or stably expressed in a cell culture system, or alternatively, a transgenic animal model.

Another aspect of the technology described herein provides a method of transducing a population of mammalian cells with a ceDNA vector for antibody or fusion protein production as disclosed herein. In an overall and general sense, the method includes at least the step of introducing into one or more cells of the population, a composition that comprises an effective amount of one or more of the ceDNA vectors for antibody or fusion protein production as disclosed herein.

Additionally, the present invention provides compositions, as well as therapeutic and/or diagnostic kits that include one or more of the disclosed ceDNA vectors for antibody or fusion protein production as disclosed herein or ceDNA compositions, formulated with one or more additional ingredients, or prepared with one or more instructions for their use.

A cell to be administered a ceDNA vector for antibody or fusion protein production as disclosed herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

A. ceDNA Vectors for Commercial Antibody or Fusion Protein Production

In some embodiments, the ceDNA vectors as disclosed herein can be used in the production of antibodies or fusion proteins in a commercial setting, for example, using a bioreactor or for production in a desired host.

For example, cells comprising a ceDNA vector for antibody or fusion protein production as disclosed herein can be used for commercial production of antibodies or fusion proteins, e.g., serving as a cell source for small or large scale biomanufacturing of antibodies or fusion proteins. In alternative embodiments, a ceDNA vector for antibody or fusion protein production as disclosed herein is introduced into cells in a host non-human subject, for in vivo production of antibodies or fusion proteins, including small scale production as well as for commercial large scale antibody or fusion protein production. For example, in some embodiments, the ceDNA vectors described herein can be used to produce antibodies or fusion proteins in vivo, for example, in rats, mice, horses, goats, etc. by use of ascites tumors.

In some embodiments, the ceDNA vectors encoding an antibody or fusion protein can be used to generate a chimeric antigen receptor (CAR), for example, which can then be used in generating CAR T cells. CARs are fusion proteins of a selected single-chain fragment variable from a specific monoclonal antibody and one or more T-cell receptor intracellular signaling domains. This T-cell genetic modification may occur using ceDNA vectors as described herein. Thus, it is specifically contemplated herein that a ceDNA vector expressing a chimeric antigen receptor can be administered to e.g., an ex vivo T cell to engineer a CAR T cell for the treatment of cancers, such as, but not limited to leukemia, breast cancer, lung cancer, ovarian cancer and the like.

### B. Antibody or Fusion Protein Production and Purification

The ceDNA vectors disclosed herein are to be used to produce antibodies or fusion proteins either in vitro or in vivo. The antibodies or fusion proteins produced in this manner can be isolated, tested for a desired function, and purified for further use in research or as a therapeutic treatment. Each system of antibody or fusion protein production has its own advantages/disadvantages. While antibodies or fusion proteins produced in vitro can be easily purified and can produce antibodies or fusion proteins in a short time, antibodies or fusion proteins produced in vivo can have post-translational modifications, such as glycosylation.

Conventional techniques for generating antibodies, such as immunization and library display (e.g., phage display), can be adapted by using ceDNA in place of a traditional vector (e.g., plasmid, virus etc) to encode an antibody or antibody component. In addition, ceDNA vectors as described herein can be replace traditional vectors in bioreactors, bioreactor generation or in the production of antibodies in a desired host, cell, tissue or organ. Such techniques are known to those of skill in the art and are not described in detail herein.

The ceDNA vectors described herein can be used to express a desired antibody in a hybridoma cell line. Methods for generation of hybridoma cell lines are known in the art. For large scale antibody production, the hybridoma cells can be grown in either a static or agitated cell suspension culture, a roller-bottle culture, or in a bioreactor (e.g., hollow fiber bioreactor). Membrane-based culture systems can also be used to produce antibodies in vitro, where the cell culture is separated from the nutrients by way of a special gassing membrane that enhances oxygen and gas transfer. Alternatively, a matrix-based culture system where the hybridoma cells are immobilized on a matrix and continuously supplied with fresh culture medium.

Antibodies produced using ceDNA vectors can be purified using any method known to those of skill in the art, for example, ion exchange chromatography, affinity chromatography, precipitation, or electrophoresis.

An antibody produced by the methods and compositions described herein can be tested for binding to the desired target protein.

### XIII. Various Other Embodiments

In some embodiments, the present application may be defined in any of the following paragraphs:

1. A DNA vector comprising at least one heterologous nucleic acid sequence encoding at least one transgene thereof operably linked to a promoter positioned between two AAV inverted terminal repeat sequences (ITRs), where the ITRs can optionally be the same or different ITRs, and where they are different ITRs, one of the ITRS comprising a functional AAV terminal resolution site and a Rep binding site, and one of the ITRs comprising a deletion, insertion, or substitution relative to the other ITR; wherein the transgene is an antibody or fragment thereof or a fusion protein; and wherein the DNA when digested with a restriction enzyme having a single recognition site on the DNA vector has the presence of characteristic bands of linear and continuous DNA as compared to linear and non-continuous DNA controls when analyzed on a non-denaturing gel.

2. The vector of paragraph 1, wherein the antibody is a full-length antibody or a fragment thereof.

3. The vector of paragraph 2, wherein the antibody is a monoclonal antibody, a single chain antibody, a Fab′ fragment or a single domain antibody (dAb).

4. The vector of any one of paragraphs 1-3, wherein the DNA vector contains a promoter operably linked to a first transgene encoding a secretory sequence and a heavy chain protein and a second promoter operably linked to a second transgene encoding a light chain protein.

5. The vector of any one of paragraphs 14, wherein the transgene encodes a fusion protein, wherein the fusion protein is a single chain variable fragment (scFv).

6. The vector of any one of paragraphs 1-5, wherein the antibody is an antibody selected from Table 1-5.

7. The vector of any one of paragraphs 1-6, wherein the ITR comprises a functional terminal resolution site and a Rep binding site is a wild-type AAV ITR, or wherein the two ITRs are symmetrical or substantially symmetrical, or wherein the two ITRs are asymmetrical, or wherein the two ITRs are selected from any listed in Tables 7, 9A, 9B and 10.

8. The vector of paragraph 1, wherein the antibody is aducanumab.

9. The vector of any one of the preceding paragraphs, wherein the antibody is a human or humanized antibody.

10. The vector of any one of the preceding paragraphs, wherein the antibody is an IgG, IgA, IgD, IgM, or IgE antibody.

11. A method for expressing an antibody in a cell or population thereof comprising administering to the cell or population thereof an effective amount of the DNA vector of paragraphs 1-10 and culturing the cell or population thereof under conditions to express the antibody in the cell.

12. The method of paragraph 11, wherein the DNA vector or ceDNA vector is administered in combination with a pharmaceutically acceptable carrier.

13. The method of paragraph 11 or 12, wherein the antibody or fragment thereof is secreted from the cell.

14. The method of any one of paragraphs 11-13, wherein the antibody or fragment thereof is retained intracellularly as an intrabody.

15. The method of any one of paragraphs 11-14, wherein the cell is a mammalian cell.

16. The method of paragraph 15, wherein the cell is a human cell.

17. The method of paragraph 15, wherein the antibody is isolated from the cell and purified.

18. A method for delivering a therapeutic antibody to a subject, the method comprising: administering to a subject, a composition comprising a DNA vector of paragraphs 1-10.

19. A method for treating disease in a subject, the method comprising: administering to a subject, a composition comprising a ceDNA vector of paragraphs 1-10, thereby expressing the therapeutic antibody in the subject and treating the disease.

20. The method of paragraph 18 or 19, wherein the ceDNA vector is administered in combination with a pharmaceutically acceptable carrier.

21. The method of any one of paragraphs 18-20, wherein the therapeutic antibody is secreted from a cell in which it was expressed.

22. The method of any one of paragraphs 18-22, wherein the therapeutic antibody is retained in a cell in which it was expressed.

23. The method of any one of paragraphs 11-17, wherein the cell or population thereof is cultured in a bioreactor.

24. A composition comprising a vector according to any one of paragraphs 1-10 for use in the treatment of a disease in a subject.

25. Use of a composition comprising a vector according to any one of paragraphs 1-10 in the treatment of a disease in a subject.

26. Use of a composition comprising a vector according to any one of paragraphs 1-10 in the preparation of a medicament for the treatment of a disease in a subject.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing ceDNA Vectors Using an Insect Cell-Based Method

Production of the ceDNA vectors using a polynucleotide construct template is described in Example 1 of PCT/US18/49996, which is incorporated herein in its entirety by reference. For example, a polynucleotide construct template used for generating the ceDNA vectors of the present invention can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector.

An exemplary method to produce ceDNA vectors is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC (SEQ ID NO: 123) and R4 (Pac) TTAATTAA (SEQ ID NO: 124) enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (080dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

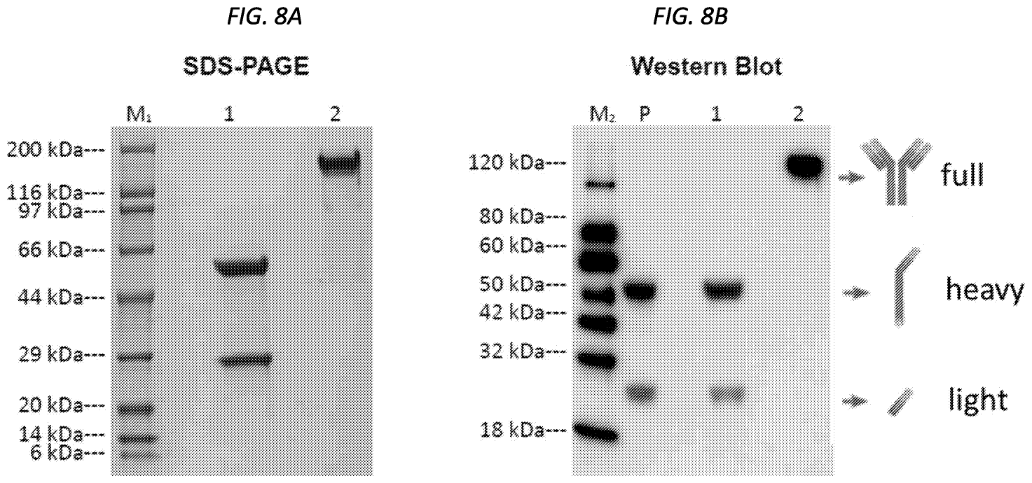

A “Rep-plasmid” as disclosed in FIG. 8A of PCT/US18/49996, which is incorporated herein in its entirety by reference, was produced in a pFASTBAC™-Dual expression vector (ThermoFisher) comprising both the Rep78 (SEQ ID NO: 131 or 133) and Rep52 (SEQ ID NO: 132) or Rep68 (SEQ ID NO: 130) and Rep40 (SEQ ID NO: 129). The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4D and 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN® G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g. Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

For comparative purposes, Example 1 describes the production of ceDNA vectors using an insect cell based method and a polynucleotide construct template, and is also described in Example 1 of PCT/US18/49996, which is incorporated herein in its entirety by reference. For example, a polynucleotide construct template used for generating the ceDNA vectors of the present invention according to Example 1 can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector.

An exemplary method to produce ceDNA vectors in a method using insect cell is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC (SEQ ID NO: 123) and R4 (Pac) TTAATTAA (SEQ ID NO: 124) enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (080dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

A “Rep-plasmid” was produced in a pFASTBAC′-Dual expression vector (ThermoFisher) comprising both the Rep78 (SEQ ID NO: 131 or 133) or Rep68 (SEQ ID NO: 130) and Rep52 (SEQ ID NO: 132) or Rep40 (SEQ ID NO: 129). The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm. The purified ceDNA vectors can be assessed for proper closed-ended configuration using the electrophoretic methodology described in Example 5.

### Example 2: Synthetic ceDNA Production Via Excision from a Double-Stranded DNA Molecule

Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector comprises a regulatory switch as described herein.

For illustrative purposes, Example 2 describes producing ceDNA vectors as exemplary closed-ended DNA vectors generated using this method. However, while ceDNA vectors are exemplified in this Example to illustrate in vitro synthetic production methods to generate a closed-ended DNA vector by excision of a double-stranded polynucleotide comprising the ITRs and expression cassette (e.g., heterologous nucleic acid sequence) followed by ligation of the free 3′ and 5′ ends as described herein, one of ordinary skill in the art is aware that one can, as illustrated above, modify the double stranded DNA polynucleotide molecule such that any desired closed-ended DNA vector is generated, including but not limited to, doggybone DNA, dumbbell DNA and the like. Exemplary ceDNA vectors for production of antibodies or fusion proteins that can be produced by the synthetic production method described in Example 2 are discussed in the sections entitled “III ceDNA vectors in general”. Exemplary antibodies and fusion proteins expressed by the ceDNA vectors are described in the section entitled “IIC Exemplary antibodies and fusion proteins expressed by the ceDNA vectors”.

The method involves (i) excising a sequence encoding the expression cassette from a double-stranded DNA construct and (ii) forming hairpin structures at one or more of the ITRs and (iii) joining the free 5′ and 3′ ends by ligation, e.g., by T4 DNA ligase.

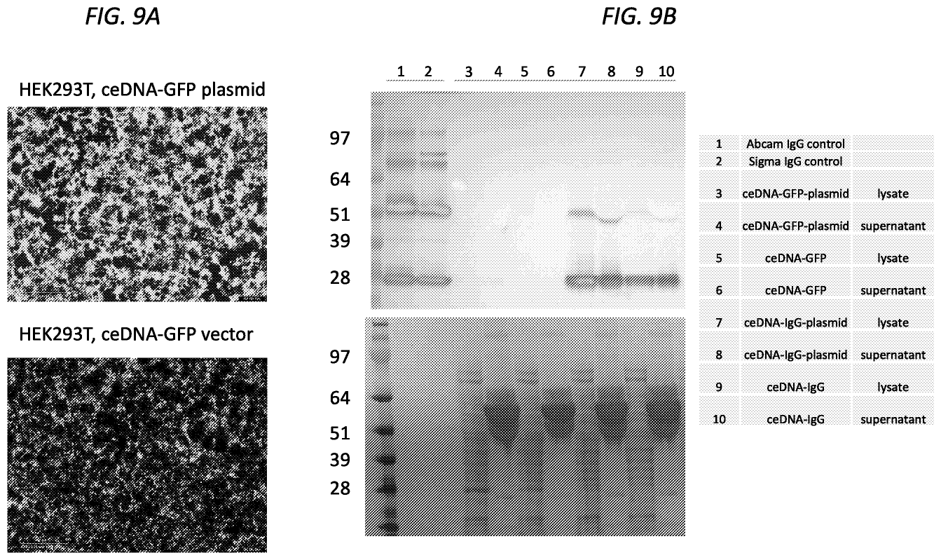

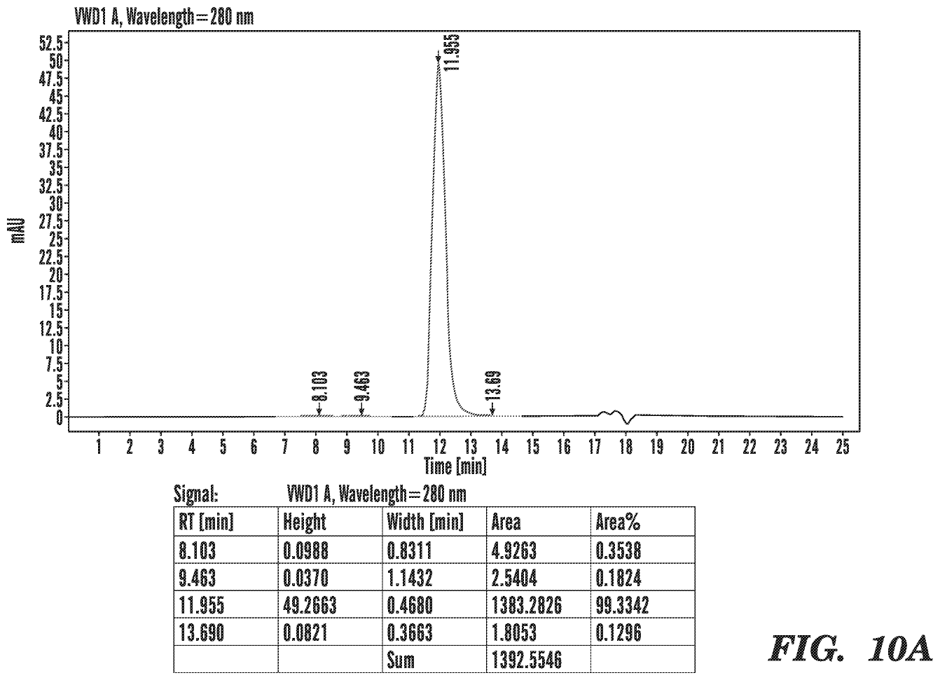

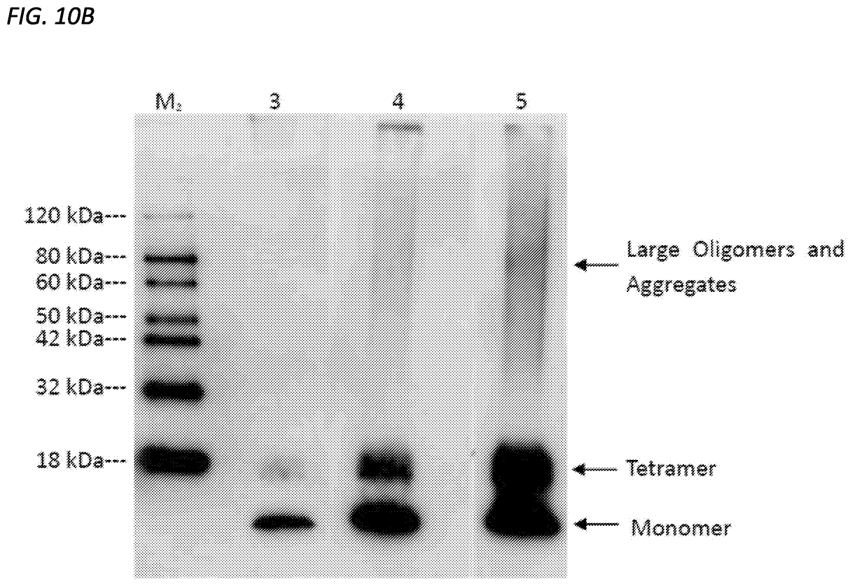

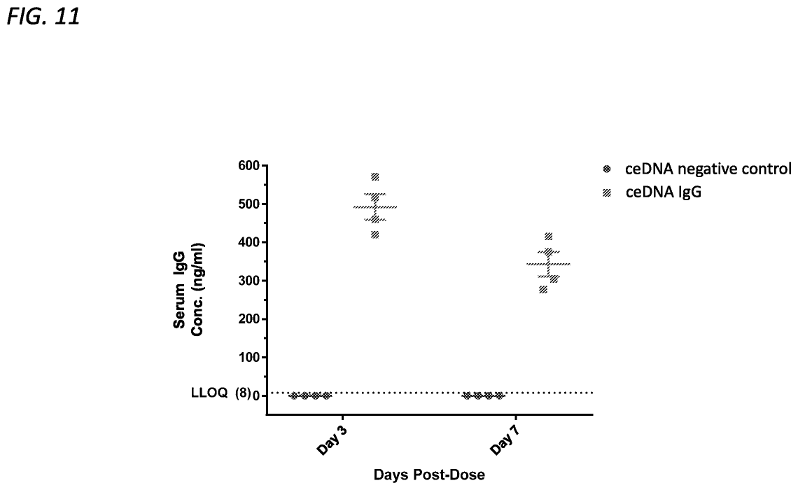

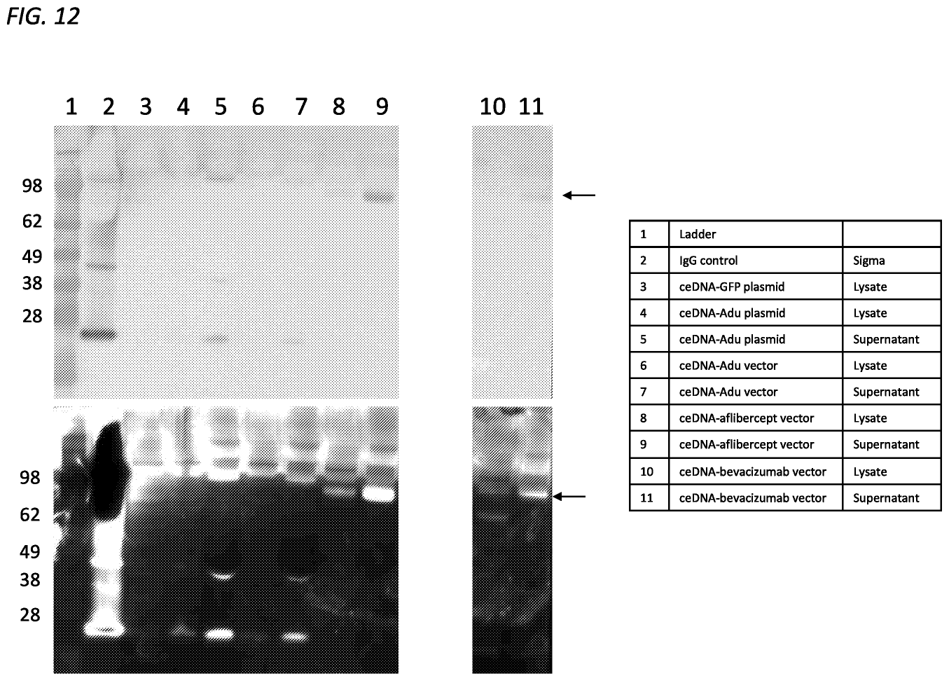

The double-stranded DNA construct comprises, in 5′ to 3′ order: a first restriction endonuclease site; an upstream ITR; an expression cassette; a downstream ITR; and a second restriction endonuclease site. The double-stranded DNA construct is then contacted with one or more restriction endonucleases to generate double-stranded breaks at both of the restriction endonuclease sites. One endonuclease can target both sites, or each site can be targeted by a different endonuclease as long as the restriction sites are not present in the ceDNA vector template. This excises the sequence between the restriction endonuclease sites from the rest of the double-stranded DNA construct (see FIG. 9 of PCT/US19/14122). Upon ligation a closed-ended DNA vector is formed.

One or both of the ITRs used in the method may be wild-type ITRs. Modified ITRs may also be used, where the modification can include deletion, insertion, or substitution of one or more nucleotides from the wild-type ITR in the sequences forming B and B′ arm and/or C and C′ arm (see, e.g., FIGS. 6-8 and 10 FIG. 11B of PCT/US19/14122), and may have two or more hairpin loops (see, e.g., FIGS. 6-8 FIG. 11B of PCT/US19/14122) or a single hairpin loop (see, e.g., FIG. 10A-10B FIG. 11B of PCT/US19/14122). The hairpin loop modified ITR can be generated by genetic modification of an existing oligo or by de novo biological and/or chemical synthesis.

In a non-limiting example, ITR-6 Left and Right (SEQ ID NOS: 111 and 112), include 40 nucleotide deletions in the B-B′ and C-C′ arms from the wild-type ITR of AAV2. Nucleotides remaining in the modified ITR are predicted to form a single hairpin structure. Gibbs free energy of unfolding the structure is about −54.4 kcal/mol. Other modifications to the ITR may also be made, including optional deletion of a functional Rep binding site or a Trs site.

### Example 3: ceDNA Production Via Oligonucleotide Construction

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

As disclosed herein, the ITR oligonucleotides can comprise WT-ITRs (e.g., see FIG. 3A, FIG. 3C), or modified ITRs (e.g., see, FIG. 3B and FIG. 3D). (See also, e.g., FIGS. 6A, 6B, 7A and 7B of PCT/US19/14122, which is incorporated herein in its entirety). Exemplary ITR oligonucleotides include, but are not limited to SEQ ID NOS: 134-145 (e.g., see Table 7 in of PCT/US19/14122). Modified ITRs can include deletion, insertion, or substitution of one or more nucleotides from the wild-type ITR in the sequences forming B and B′ arm and/or C and C′ arm. ITR oligonucleotides, comprising WT-ITRs or mod-ITRs as described herein, to be used in the cell-free synthesis, can be generated by genetic modification or biological and/or chemical synthesis. As discussed herein, the ITR oligonucleotides in Examples 2 and 3 can comprise WT-ITRs, or modified ITRs (mod-ITRs) in symmetrical or asymmetrical configurations, as discussed herein.

### Example 4: ceDNA Production Via a Single-Stranded DNA Molecule

Another exemplary method of producing a ceDNA vector using a synthetic method is provided in Example 4 of PCT/US19/14122, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

An exemplary single-stranded DNA molecule for production of a ceDNA vector comprises, from 5′ to 3′:
a sense first ITR;
a sense expression cassette sequence;
a sense second ITR;
an antisense second ITR;
an antisense expression cassette sequence; and
an antisense first ITR.

A single-stranded DNA molecule for use in the exemplary method of Example 4 can be formed by any DNA synthesis methodology described herein, e.g., in vitro DNA synthesis, or provided by cleaving a DNA construct (e.g., a plasmid) with nucleases and melting the resulting dsDNA fragments to provide ssDNA fragments.

Annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated by one of ordinary skill in the art.

The free 5′ and 3′ ends of the annealed molecule can be ligated to each other, or ligated to a hairpin molecule to form the ceDNA vector. Suitable exemplary ligation methodologies and hairpin molecules are described in Examples 2 and 3.

### Example 5: Purifying and/or Confirming Production of ceDNA

Any of the DNA vector products produced by the methods described herein, e.g., including the insect cell based production methods described in Example 1, or synthetic production methods described in Examples 2-4 can be purified, e.g., to remove impurities, unused components, or byproducts using methods commonly known by a skilled artisan; and/or can be analyzed to confirm that DNA vector produced, (in this instance, a ceDNA vector) is the desired molecule. An exemplary method for purification of the DNA vector, e.g., ceDNA is using Qiagen Midi Plus purification protocol (Qiagen) and/or by gel purification,

The following is an exemplary method for confirming the identity of ceDNA vectors.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the purified DNA with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4C and 4D, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a ceDNA vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4E).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBETAE with 1×SYBR Gold. Bands can then be visualized with e.g. Thermo Fisher, SYBR® Gold Nucleic Acid Gel Stain (10,000× Concentrate in DMSO) and epifluorescent light (blue) or UV (312 nm). The foregoing gel-based method can be adapted to purification purposes by isolating the ceDNA vector from the gel band and permitting it to renature.

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 6: Antibody or Fusion Protein Production from a ceDNA Plasmid

In order to demonstrate the ability of a ceDNA vector to express an antibody, ceDNA plasmids encoding the monoclonal antibody aducanumab were generated.

Aducanumab is a human monoclonal antibody being studied for the treatment of Alzheimer's disease by targeting aggregated forms of beta-amyloid protein. FIG. 6A shows an exemplary plasmid that was generated and determined to express full length aducanumab. FIG. 6B is a schematic of a ceDNA vector that can be obtained using the ceDNA plasmid of FIG. 6A. The expression cassette of FIG. 6B shows a dual promoter system where a different promoter is used for each of the heavy chain and light chains of the aducanumab antibody. The ceDNA plasmid that was used to produce aducanumab antibody includes a unique combination of promoters for expression of the heavy and light chain that results in the proper ratios of heavy and light chains for the formation of functional antibody. One of ordinary skill in the art will appreciate how to select promoters for each of the light and heavy chains of a desired antibody to effect production of the desired ratios of expressed heavy and/or light chains to foster efficient formation of the intact antibody.

ceDNA plasmid referred to as “pFBdual-ceDNA-Aducanumab plasmid”, or “ceDNA-IgG-plasmid” as shown in FIG. 6A, was prepared as described in Example 1 and used for transient transfection of 293 cells. 293-6E cells were grown in serum-free FreeStyle™ 293 Expression Medium (Life Technologies, Carlsbad, Calif., USA). The cells were maintained in Erlenmeyer Flasks (Corning Inc., Acton, Mass.) at 37° C. with 5% CO₂ on an orbital shaker (VWR Scientific, Chester, Pa.). One day prior to transfection, the cells were seeded at an appropriate density in Corning Erlenmeyer Flasks. On the day of transfection, DNA and transfection reagent were mixed at an optimal ratio and then added into the flask with cells ready for transfection. The recombinant plasmid encoding target protein was transiently transfected into suspension 293-6E cell cultures. Cell density and viability on day 2, day 4 and day 5 post-transfection are listed in Table 13. The cell culture supernatant samples collected on day 2, day 4 and day 5 were used for the protein expression evaluation. The cell culture supernatant harvested on day 6 post transfection was used for purification.

TABLE 13
Cell density and viability of ceTTX-IgG1-Adu in 293-6E cells

| Sample | Density (×10⁶ cells/mL) | Viability (%) |
|---|---|---|
| U8948CK300-1 Day 2 | 1.80 | 95.36 |
| U8948CK300-1 Day 4 | 2.36 | 83.98 |
| U8948CK300-1 Day 5 | 2.20 | 72.28 |

Expression and purification of antibody: To estimate the protein expression level, cell culture supernatants were collected on day 2, 4 and 5 post-transfection and were analyzed by SDS-PAGE and Western blot (data not shown). For protein purification, the cell culture broth was centrifuged and the supernatant filtered. Filtered supernatant was loaded onto Monofinity A Resin Prepacked Column 1 ml (GenScript) at 1.0 ml/min, followed by washing and elution with appropriate buffers. The eluted fractions were pooled and buffer exchanged to PBS pH7.2. The purified protein was analyzed by SDS-PAGE (FIG. 8A), Western blot (FIG. 8B) and SEC-HPLC (data not shown) by using standard protocols for molecular weight, yield and purity measurements.

In summary, aducanumab was expressed from the ceDNA vector produced from the pFBdual-ceDNA-Aducanumab plasmid in 293-6E cells grown in suspension culture. The antibody was expressed at a level that could be discerned using SDS-PAGE analysis (FIG. 8A). After one step purification, the antibody was detected with estimated molecular weights of ˜55 kDa and ˜25 kDa under reducing conditions and ˜150 kDa under non-reducing conditions (FIG. 8B). These data indicate that the heavy and light chains of the antibody self-assembled into a full antibody in this system.

### Example 7: Large-Scale Production of Recombinant Antibody In Vitro

A ceDNA plasmid comprising sequences encoding the aducanumab heavy chain and light chain can be produced as previously described in Example 1 and 5 and transfected into a large-scale suspension culture of e.g. FreeStyle™ 293-F cells (R790-07, Life Technologies) that have been adapted to grow in serum-free conditions. Suspension and serum-free adapted FreeStyle™ 293-F cells (Life Technologies) are cultured in FreeStyle™ 293 Expression Medium (Life Technologies) in 125 mL sterile Erlenmeyer flasks with vented caps (Sigma) at densities between 1×105-5×105 viable cells/mL, rotating at 135 rpm on an orbital shaker platform. Each 125 mL flask containing 30 mL of cells at 1×10⁶ viable cells/mL is transfected with ceDNA Fugene6 transfection reagent (3:1 Fugene6:ceDNA). according to the manufacturer's instructions. 24 h post-transfection, selection (50 mg/mL) is added to cells and the cells are maintained under selection for 2 weeks at densities between 2×105-5×105 viable cells/mL, followed by expansion into a 1 L shaker flask (Sigma), 1 L spinner bottle (Sigma) or 5 L WAVE bioreactor (GE Healthcare). Samples are collected every 48 h and IgG expression levels are determined by anti-human IgG ELISA. At peak expression cultured supernatants are harvested, centrifuged at 1000×g for 15 minutes, passed over 0.45 mm filters (Sartorius) and stored at 4° C. with 0.1% sodium azide (Sigma) until use. Antibodies can be purified by affinity chromatography with a 5 mL HiTrap Protein-G HP column (GE Healthcare) using an ÄKTA Prime system (GE Healthcare) and 0.2 μm filtered buffers. For Example, the column is equilibrated with 10 Column Volumes (CV) of phosphate buffered saline (PBS) washing buffer (pH 7.0) and the supernatant loaded at a flow rate of 2 mL/min, followed by 10 CV of washing buffer. Antibody is eluted with 0.2 M Glycine buffer (pH 2.3) and 2.5 mL fractions were collected into tubes containing 0.5 mL 1 M Tris-HCl pH 8.6 for neutralization.

### Example 8: Making of ceDNA Vector Expressing Full Length Aducanumab and ceDNA Expression of Antibody In Vitro

ceDNA plasmid or ceDNA vector that expresses aducanumab or GFP was prepared as described in Examples 1 and 5, using ceDNA Adu-Full-IgG1 plasmid (pFBdual-ceDNA-Aducanumab plasmid or ceDNA-IgG-plasmid) (FIG. 6A) as prepared in Example 1. Yields of produced and purified ceDNA vector (referred to as “ceDNA IgG”) or ceDNA plasmid were determined based on UV absorbance at 260 nm. ceDNA vector or ceDNA plasmid was transfected into HEK293T cells using the Lipofectamine™ 3000 transfection reagent (Invitrogen) according to the manufacturer's instructions. After 72 hours, cells were lysed in RIPA buffer, whole cell supernatant was collected and concentrated using filters (Amicon). The resulting antibody production was examined by western blot. Cell supernatants and lysate samples were normalized in concentration so that equal amounts of protein were loaded. Prepared samples were boiled at 70° C. in a heat block for 10 minutes, and then placed on ice. Samples were run on an SDS-PAGE gel at 200V for 32 min and then transferred to nitrocellulose membrane using standard techniques. Commercially available human IgG was used as a positive control (Abcam, Sigma). The membrane was blocked with blocking buffer (Odyssey) at room temperature for 30 min. Protein bands were visualized by staining the membrane in Ponceau S stain for 5 min, followed by washing with distilled water and destaining with TBST. The membrane was then probed overnight at 4° C. with gentle agitation with primary anti-human IgG antibody (Genscript) conjugated to horseradish peroxidase, diluted 1:5000 in blocking buffer. The blot was then washed three times for 5 min each time with TBST and developed with the ECL kit (SuperSignal West Femto Substrate) and imaged using a gel imaging system (Syngene Box Mini).

The transfection efficiency of the Lipofectamine™ 3000 transfection procedure was assessed by examining the fluorescence of the 293T cells transfected with the ceDNA-GFP plasmid (FIG. 9A, top panel) or ceDNA-GFP vector (FIG. 9A, bottom panel). Both samples had significant fluorescence as shown in FIG. 9A, indicating that transfection was successful in both cases. Despite significant protein content in each sample (FIG. 9B, lower panel), the presence of expressed aducanumab was detected only in the samples from cells transfected with either the ceDNA-IgG plasmid or ceDNA-IgG constructs (FIG. 9B, upper panel, with both heavy and light chains observed). This indicated that ceDNA vector expressed aducanumab in 293T cells.

### Example 9: Confirmation of Identity of the Expressed Antibody

Plasmids comprising the nucleic acid of interest encoding aducanumab were prepared for expression in human embryonic kidney cells (HEK293-6E) cells. HEK293-6E cells were grown in serum free medium (FreeStyle™ 293 Expression Medium, Thermo Fisher Scientific). The cells were maintained in Erlenmeyer flasks at 37° C. with 5% CO₂ on an orbital shaker. One day prior to transfection, the cells were seeded at an appropriate density in Erlenmeyer flasks. On the day of transfection, DNA and transfection reagent were mixed at an optimal ratio and then added into the flask with cells ready for transfection. The recombinant plasmid encoding target protein was transiently transfected into suspension HEK293-6E cell cultures. The cell culture supernatants collected on day 6 were used for purification.

The cell culture broth was centrifuged, filtered, and loaded onto an affinity purification column (Monofinity A Resin™ Prepacked Column, GenScript) at an appropriate flow rate. After washing and elution, the eluted fractions were pooled and buffer exchanged to the final formulation buffer. Purified protein was analyzed by (a) SDS-polyacrylamide gel electrophoresis under reducing and non-reducing conditions, (b) western blotting with goat-anti-human IgG-HRP (GenScript) and goat anti-human kappa-HRP as primary antibodies, with chemiluminescent detection, and (c) size exclusion chromatography using TSKgel G3000SWxl (Tosoh Bioscience) high performance liquid chromatography to determine the molecular weight and purity. Protein concentration was determined by absorption at A260/280. The results are shown in FIG. 10A. HPLC analysis revealed a single peak (FIG. 10A). When samples of this protein were run on an SDS-PAGE gel (FIG. 8A), a single band was evident in non-reducing conditions (Lane 2) while two bands were seen when the sample was subjected to reducing conditions (Lane 1). This is consistent with the protein being an antibody, where the light chain and heavy chain subunits remain disulfide-bound under nonreducing conditions and migrate as a single band, but separate under reducing conditions into their separate subunits, migrating as two bands. The western blot showed a similar banding pattern (FIG. 8B), and further confirmed the presence of antibody since the detection was by primary antibodies specific for human IgG and human kappa light chain.

The ability of the purified aducanumab to recognize its ligand beta-amyloid (142) (“Abeta”) was assessed by ELISA. Amyloid was aggregated in vitro and adhered to a plate followed by exposure to the aducanumab or control antibodies at various concentrations and times and then colorimetric exposure. Synthetic Aβ1-42 peptide (AnaSpec, Fremont, Calif., USA) was reconstituted in hexafluoro-isopropanol at a concentration of 1 mg/ml, air dried and vacuum concentrated to form a film. The film was dissolved in DMSO and AB42 oligomers and AB42 fibrils were prepared by diluting DMSO reconstituted monomeric into PBS at a concentration of 100 μg/mL and incubating at 37° C. for at least 3 days and 1 week, respectively. ELISA plates were coated with aggregated amyloid.

Briefly, the Abeta was prepared and the Western blot run and stained according to the methods described by Stine et al., Methods Mol. Biol. (2011) 670: 13-32. Equivalent amounts of Abeta were run on the gel in each of lanes 3, 4, and 5. After transfer to nitrocellulose, each lane was separated and probed with different primary antibodies: the purified aducanumab described herein in Lane 3, purified anti-beta-amyloid (17-24) (BioLegend, clone 4G8) in Lane 4, and purified anti-beta-amyloid (1-16) (BioLegend, clone 6E10) in Lane 5. After washing, each was probed with goat anti-human IgG-HRP (Lane 3) (GenScript) or goat anti-mouse IgG-HRP (Lanes 4 and 5) (GenScript) as secondary antibody and developed with the HRP substrate. The results are shown in FIG. 10B. The plasmid-expressed aducanumab bound to the nitrocellulose-immobilized Abeta monomer, as did the other two anti-Abeta antibodies, indicating that the purified aducanumab was able to recognize its ligand as expected.

### Example 10: In Vivo Expression of Antibody in Wild-Type Mice

ceDNA vector with a wild-type left ITR and a truncation mutant right ITR and having a transgene region encoding the aducanumab heavy chain and light chain, each under the control of its own EF1 promoter, was prepared and purified as described above in Examples 1 and 5 (“ceDNA IgG vector”). The ceDNA IgG vector or a ceDNA control vector comprising a luciferase transgene under control of the liver-specific hAAT promoter were administered to male C57bl/6J mice of approximately 6 weeks of age. The unencapsulated ceDNA vectors were dosed at 0.005 mg per animal (4 animals per group) by hydrodynamic intravenous injection via lateral tail vein in a volume of 2.2 mL. Blood samples were collected from each treated animal on days 3, 7, 14, 21, and at terminal day 28. The presence of expressed aducanumab in the serum samples was measured by ELISA using a polyclonal anti-human immunoglobulin antibody that recognizes human antibodies of any specificity in the commercially available Discovery Human/NHP IgG kit following the manufacturer's instructions (Meso Scale Discovery).

As shown in FIG. 11, human antibodies were readily detected in day 3 and 7 serum samples from mice treated with ceDNA IgG vector, but were not observed in mice treated with ceDNA expressing luciferase and not a human antibody. The maximum level of serum expression observed in these two time points for this particular vector was approximately 500 ng/mL.

### Example 11: In Vivo Expression of Antibody in an Alzheimer Disease Mouse Model

ceDNA vector with sequences encoding the aducanumab heavy chain and light chain can be produced as previously described in Examples 1 and 5. ceDNA vector will be formulated with lipid nanoparticles and administered to Tg2576 mice (Kawarabayashi et al, J. Neurosci 21(2):372-381 (2001)) and normal mice. The LNP-ceDNA vectors are administered to respective mice at doses between 0.3 and 5 mg/kg in 1.2 mL volume. Each dose is to be administered via i.v. hydrodynamic administration, or will be administered for example by intraperitoneal injection. Administration to normal mice serves as a control and also can be used to detect the presence and quantity of aducanumab mAb. Both in vitro and ex vivo binding assays (ELISA with aggregated amyloid and brain slice IHC respectively will be performed, as described herein and in Example 6.

Following an acute dosing, e.g. a., single dose of LNP-TTX-Adu ceDNA plasma and brain concentrations will be determined by ELISA at various time points, e.g., at 10, 20, 30, 40, 50, 1000 and 200 days or more, etc. Specifically, frozen brains are homogenized in 10 volumes (10 mL/g of wet tissue) of a solution containing 50 mM NaCl, 0.2% diethylamine (DEA), with protease inhibitors, and sonicated for approximately 15-20 s on ice. The samples are centrifuged at 100,000 g for 30 min at 4° C. and the supernatant retained as the DEA extracted soluble Aβ fraction. The remaining pellets are resuspended in 10 volumes of 5M guanidine-hydrochloride (Gu-HCl), sonicated, and centrifuged as above. The resultant supernatant is retained as the guanidine-extracted insoluble Aβ fraction, and the remaining pellet is discarded. For plasma and brain Antibody concentrations, 96 well microplates (Nunc Maxisorp, Corning Costar) are coated with Aβ(1-42) peptide (Aβ42) at a concentration of 5 ug/mL in cold coating buffer overnight at 4° C. Plasma or DEA extracted (i.e., detergent-free) brain homogenates samples is diluted to final working concentrations and incubated for 2 hours at room temperature. Binding is determined using a reporter antibody, e.g. horseradish peroxidase (HRP)-conjugated goat anti-human polyclonal antibody (Jackson ImmunoResearch) followed by measurement of HRP activity using the substrate TMB. Concentrations are determined by comparison to a standard curve generated using purified antibodies. Alternatively, Ab in Plasma is confirmed using a WESTERN blot as described in

In vivo binding of antibody to Aβ deposits after a single dose in Tg2576 mice can also be determined using a biotinylated anti-human secondary antibody, and can be compared to the staining with the pan-Aβ antibody 5F3 performed ex vivo on a consecutive section as a positive control. Tissues are formalin-fixed paraffin embedded, sectioned at 5μπι, deparaffinized and an EDTA-borate based heat-induced epitope retrieval (Ventana CC1) can be used. Subsequently, a goat anti-human secondary antibody can be applied to tissues at a 1:500 dilution and tissues stained with hematoxylin. It is expected that systemically administered LNP-TTX-Adu ceDNA will be expressed in plasma and brain and bind to parenchymal amyloid plaques with high affinity.

### Example 12: Assay for Reduction of Amyloid Burden Following Dosing with ceDNA

Efficacy of ceDNA expressed aducanumab in reducing amyloid burden will be evaluated following chronic dosing in Tg2576 transgenic mice. For example, doses in the range of 0.3, 1, 3, 10, to 30 mg/kg etc., or PBS, will be administered at a given time frame, e.g. weekly or monthly. Plasma and brain drug levels will be measured by ELISA as described above in Example 8. Plasma samples are collected 24 h-72 hrs after the final dose, and antibody levels will be measured to determine dose response.

Total brain amyloid load in the cortex and hippocampus will be revealed by immunohistochemistry. Specifically, brains are dissected and fixed by immersion in 10% neutral buffered formalin for 48 to 72 h. Fixed brains are then processed and embedded in a horizontal orientation. Each block is sectioned until the hippocampus was identified at which point 300 consecutive 5μιηsections (3 sections per slide) are obtained. For both 6E10 and Thioflavin-S staining, every 14^{th} slide was stained (approximately 1 section every 225 μm). Immunohistochemistry to define brain amyloid will use e.g. mouse anti-Aβ 1-16 monoclonal antibody (Clone 6E10, Covance, Princeton, N.J.) as the primary antibody at a 1:750 dilution, and the Ultramap anti-mouse Alkaline Phosphatase Kit (Ventana Medical Systems, Tucson, Ariz.) and quantified using the VISIOPHARM™ software as described herein. Slides are pretreated with 88% formic acid solution, prior to being placed on a Ventana Discovery XT immunostainer. Slides are counterstained with Ventana Hematoxylin (Ventana Medical Systems, Tucson, Ariz.), coverslipped and air dried overnight.

After 6E10 immunostaining, slides are scanned with an Aperio XT (Aperio Technologies, Inc., Vista, Calif.) whole slide imaging system at 20× magnification following manufacturer's instructions. Digital images can then be reviewed and manually annotated as separate masks and then analyzed using an algorithm written with VISIOPHARM′\hat{ } software. The algorithm determines the area of the annotated hippocampus or cortex and the areas of parenchymal and vascular amyloid in these anatomic regions at 10× virtual magnification. Training of the software is, for example, performed on a set of 50 slides. Slides are also stained with Thioflavin-S (Thio-S) as described in (Bussiere et ah, Am J Pathol 165:987-995 (2004)), and coverslipped with VECTASHIELD® Mounting Media with DAPI (Vector Laboratories Burlingame, Calif.). After Thioflavin-S staining, slides are e reviewed and scanned with an imaging system, e.g. an Aperio FL (Aperio Technologies, Inc., Vista, Calif.) fluorescent whole slide imaging system at 20× magnification following manufacturer's instructions. As with 6E10, the hippocampus or cortex from are manually annotated as separate masks and then analyzed by using an algorithm written with VISIOPHARM® software and adapted for fluorescence. The algorithm determines the area of the hippocampus and the cortex and the areas of parenchymal and vascular amyloid in these anatomic regions at 10× virtual magnification. Training of the software can be performed on a set of 10 slides. It is expected that the total area occupied by stained deposits will be significantly reduced as compared to the PBS control.

### Example 13: ceDNA-Based Expression of Antibody and Fc-Fusion Protein In Vitro

To assess the ability of ceDNA to express other antibodies and immunoglobulin-like molecules aside from aducanumab, the experiments in Example 8 were repeated with two additional ceDNA constructs—one encoding the light and heavy chains of bevacizumab (antibody specifically binding to vascular endothelial growth factor (“VEGF”)) in the gene cassette and one encoding the Fc fusion protein aflibercept (a human IgG1 Fc fused to the VEGF-binding portions of the extracellular domains of human VEGF receptors 1 and 2). The results of the western blot on the non-reduced samples are shown in FIG. 12 (two images of the same blot with different exposure times of 6 seconds and 12 seconds are shown). Aducanumab was again expressed in ceDNA-Adu-plasmid and from ceDNA-Adu vector-transfected cells as had previously been found in Example 8 (lanes 5 and 7). Bevacizumab was similarly expressed in the ceDNA-bevacizumab vector-transfected cells (lane 11). Aflibercept was also expressed in the ceDNA-aflibercept vector-transfected cells (lane 9). This demonstrates that expression in cells of various IgG and immunoglobulin-like molecules from ceDNA constructs is obtainable.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A capsid-free close-ended DNA (ceDNA) vector comprising:
at least one heterologous nucleotide sequence between flanking inverted terminal repeats (ITRs), wherein the at least one heterologous nucleotide sequence encodes at least one antibody and/or fusion protein.

2. The ceDNA vector of claim 1, wherein the at least one heterologous nucleotide sequence encodes an antibody and the antibody is a full-length antibody, a Fab, a Fab′, a single-domain antibody, or a single-chain antibody (scFv).

3. (canceled)

4. The ceDNA vector of claim 2, wherein the at least one heterologous nucleotide sequence encodes a single-domain antibody or a single-chain antibody.

5. The ceDNA vector of claim 4, wherein the at least one heterologous nucleotide sequence further encodes a secretory leader sequence upstream of the single-domain antibody or single-chain antibody.

6. The ceDNA vector of claim 1, wherein:
a first heterologous nucleotide sequence encodes a heavy chain variable region and a second heterologous nucleotide sequence encodes a light chain variable region; or
a first heterologous nucleotide sequence encodes a heavy chain variable region and a heavy chain constant region or portion thereof, and a second heterologous nucleotide sequence encodes a light chain variable region and a light chain constant region or portion thereof.

7.-8. (canceled)

9. The ceDNA vector of claim 1, wherein:
the antibody is a human or humanized antibody; and/or
the antibody is an IgG, IgA, IgD, IgM, or IgE antibody.

10.-12. (canceled)

13. The ceDNA vector of claim 1, wherein the antibody binds to at least one target selected from the targets listed in Tables 2, 3A, 3B, 4, and 5.

14. The ceDNA vector of claim 1, wherein at least one heterologous nucleotide sequence encodes a fusion protein.

15.-17. (canceled)

18. The ceDNA vector of claim 1, wherein the antibody or fusion protein is selected from the antibodies and fusion proteins of Tables 1, 2, 3A, 3B, 4, or 5;
the ceDNA vector comprises one or more poly-A sites;
the ceDNA vector comprises at least one promoter operably linked to at least one heterologous nucleotide sequence; and/or
the flanking ITRs comprise a functional terminal resolution site and a Rep binding site.

19.-23. (canceled)

24. The ceDNA vector of claim 1, wherein the flanking ITRs are symmetric or asymmetric;
one or both of the ITRs are from a virus selected from a parvovirus, a dependovirus, and an adeno-associated virus (AAV);
one or both of the ITRs are wild type;
wherein the flanking ITRs are from a pair of viral serotypes shown in Table 6;
wherein one or both of the ITRs are synthetic;
wherein one or both of the ITRs are not a wild type ITR; and/or
wherein one or both of the flanking ITRs are derived from an AAV serotype AAV2.

25.-34. (canceled)

35. The ceDNA vector of claim 1, wherein one or both of the ITRs is modified by a deletion, insertion, and/or substitution in at least one of the ITR regions selected from A, A′, B, B′, C, C′, D, and D′.

36.-44. (canceled)

45. The ceDNA vector of claim 1, wherein at least one heterologous nucleotide sequence is under the control of at least one regulatory switch.

46. (canceled)

47. A method of expressing an antibody or fusion protein in a cell comprising contacting the cell with the ceDNA vector of claim 1.

48.-52. (canceled)

53. A method of treating a subject with a therapeutic antibody or therapeutic fusion protein, comprising administering to the subject a ceDNA vector of claim 1, wherein the at least one heterologous nucleotide sequence encodes the therapeutic antibody or therapeutic fusion protein.

54.-58. (canceled)

59. A pharmaceutical composition comprising the ceDNA vector of claim 1.

60. A cell containing a ceDNA vector of claim 1.

61. A composition comprising a ceDNA vector of claim 1 and a lipid.

62. The composition of claim 61, wherein the lipid is a lipid nanoparticle (LNP).

63. A kit comprising the ceDNA vector of claim 1.

64. A method of producing an antibody or fusion protein comprising culturing the cell of claim 60 under conditions suitable for producing the antibody or fusion protein.

65. (canceled)


SEQUENCE LISTING (190 sequences)

SEQ ID NO: 1 - DNA, 141 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 141 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 130 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 4 - DNA, 130 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 5 - DNA, 143 bp, Artificial Sequence
  Description of Artificial Sequence Synthetic polynucleotide
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 6 - DNA, 144 bp, Artificial Sequence
  Description of Artificial Sequence Synthetic polynucleotide
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 7 - DNA, 127 bp, Artificial Sequence
  Description of Artificial Sequence Synthetic polynucleotide
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 8 - DNA, 166 bp, Artificial Sequence
  Description of Artificial Sequence Synthetic polynucleotide
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 9 - DNA, 144 bp, Artificial Sequence
  Description of Artificial Sequence Synthetic polynucleotide
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 10 - DNA, 143 bp, Artificial Sequence
  Description of Artificial Sequence Synthetic polynucleotide
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 11 - DNA, 144 bp, Artificial Sequence
  Description of Artificial Sequence Synthetic polynucleotide
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 12 - DNA, 127 bp, Artificial Sequence
  Description of Artificial Sequence Synthetic polynucleotide
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 13 - DNA, 166 bp, Artificial Sequence
  Description of Artificial Sequence Synthetic polynucleotide
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 14 - DNA, 144 bp, Artificial Sequence
  Description of Artificial Sequence Synthetic polynucleotide
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 15 - DNA, 120 bp, Artificial Sequence
  Description of Artificial Sequence Synthetic polynucleotide
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 16 - DNA, 122 bp, Artificial Sequence
  Description of Artificial Sequence Synthetic polynucleotide
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 17 - DNA, 129 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 18 - DNA, 101 bp, Artificial Sequence
  Description of Artificial Sequence Synthetic polynucleotide
aggaacccctagtgatggagttggccactccctctctgcgcgctcgctcgctcactgaggctttgcctcagtgagcgagcgagcgcgcagctgcctgcagg

SEQ ID NO: 19 - DNA, 139 bp, Artificial Sequence
  Description of Artificial Sequence Synthetic polynucleotide
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 20 - DNA, 137 bp, Artificial Sequence
  Description of Artificial Sequence Synthetic polynucleotide
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 21 - DNA, 135 bp, Artificial Sequence
  Description of Artificial Sequence Synthetic polynucleotide
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 22 - DNA, 133 bp, Artificial Sequence
  Description of Artificial Sequence Synthetic polynucleotide
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 23 - DNA, 139 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 24 - DNA, 137 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 25 - DNA, 135 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 26 - DNA, 133 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 27 - DNA, 131 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 28 - DNA, 129 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 29 - DNA, 127 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 30 - DNA, 122 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 31 - DNA, 130 bp, Artificial Sequence; identical to SEQ ID NO: 3
  Description of Artificial Sequence Synthetic polynucleotide

SEQ ID NO: 32 - DNA, 120 bp, Artificial Sequence
  Description of Artificial Sequence Synthetic polynucleotide
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 33 - DNA, 122 bp, Artificial Sequence
  Description of Artificial Sequence Synthetic polynucleotide
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 34 - DNA, 122 bp, Artificial Sequence
  Description of Artificial Sequence Synthetic polynucleotide
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 35 - DNA, 129 bp, Artificial Sequence
  Description of Artificial Sequence Synthetic polynucleotide
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 36 - DNA, 101 bp, Artificial Sequence
  Description of Artificial Sequence Synthetic polynucleotide
cctgcaggcagctgcgcgctcgctcgctcactgaggcaaagcctcagtgagcgagcgagcgcgcagagagggagtggccaactccatcactaggggttcct

SEQ ID NO: 37 - DNA, 139 bp, Artificial Sequence
  Description of Artificial Sequence Synthetic polynucleotide
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 38 - DNA, 137 bp, Artificial Sequence
  Description of Artificial Sequence Synthetic polynucleotide
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 39 - DNA, 135 bp, Artificial Sequence
  Description of Artificial Sequence Synthetic polynucleotide
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 40 - DNA, 133 bp, Artificial Sequence
  Description of Artificial Sequence Synthetic polynucleotide
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 41 - DNA, 139 bp, Artificial Sequence
  Description of Artificial Sequence Synthetic polynucleotide
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 42 - DNA, 137 bp, Artificial Sequence
  Description of Artificial Sequence Synthetic polynucleotide
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 43 - DNA, 135 bp, Artificial Sequence
  Description of Artificial Sequence Synthetic polynucleotide
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 44 - DNA, 133 bp, Artificial Sequence
  Description of Artificial Sequence Synthetic polynucleotide
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 45 - DNA, 131 bp, Artificial Sequence
  Description of Artificial Sequence Synthetic polynucleotide
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 46 - DNA, 129 bp, Artificial Sequence
  Description of Artificial Sequence Synthetic polynucleotide
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 47 - DNA, 127 bp, Artificial Sequence
  Description of Artificial Sequence Synthetic polynucleotide
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 48 - DNA, 122 bp, Artificial Sequence; identical to SEQ ID NO: 30
  Description of Artificial Sequence Synthetic polynucleotide

SEQ ID NO: 49 - DNA, 12 bp, Artificial Sequence
  Description of Artificial Sequence Synthetic oligonucleotide
cgatcgttcgat

SEQ ID NO: 50 - DNA, 12 bp, Artificial Sequence
  Description of Artificial Sequence Synthetic oligonucleotide
atcgaaccatcg

SEQ ID NO: 51 - DNA, 12 bp, Artificial Sequence
  Description of Artificial Sequence Synthetic oligonucleotide
atcgaacgatcg

SEQ ID NO: 52 - DNA, 165 bp, Artificial Sequence
  Description of Artificial Sequence Synthetic polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 53 - DNA, 140 bp, Artificial Sequence
  Description of Artificial Sequence Synthetic polynucleotide
cccctagtgatggagttggccactccctctctgcgcgctcgctcgctcactgaggccgcccgggcaaagcccgggcgtcgggcgacctttggtcgcccggcctcagtgagcgagcgagcgcgcagagagatcactagggg

SEQ ID NO: 54 - DNA, 91 bp, Artificial Sequence
  Description of Artificial Sequence Synthetic oligonucleotide
gcgcgctcgctcgctcactgaggccgcccgggcaaagcccgggcgtcgggcgacctttggtcgcccggcctcagtgagcgagcgagcgcgc

SEQ ID NO: 55 - DNA, 91 bp, Artificial Sequence
  Description of Artificial Sequence Synthetic oligonucleotide
gcgcgctcgctcgctcactgaggccgggcgaccaaaggtcgcccgacgcccgggctttgcccgggcggcctcagtgagcgagcgagcgcgc

SEQ ID NO: 56 - DNA, 10808 bp, Artificial Sequence
  Description of Artificial Sequence Synthetic polynucleotide
aaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggcggccggcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcgcggccgcggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtatggactggacctggcgaattcttttccttgtagcagcggctactggtgcacattcattggttgagtcaggcggtggggtagtacagcctggccggtcccttaggctctcctgcgccgcgtccgggttcgcgtttagctcctacgggatgcactgggttagacaggctcctggaaagggactggaatgggttgcagtcatttggtttgacgggaccaaaaaatactatacagattccgttaaggggaggtttacaatcagtagagataatagtaaaaacacgttgtatcttcaaatgaatactctcagagccgaagatacagcagtatattactgcgcccgcgatcggggaataggcgctcggaggggaccctactatatggatgtgtggggtaaaggtacgaccgtgactgtgagctctgcgtccaccaaaggtccaagtgtttttcccctcgccccttcaagcaagtcaacctcaggcggtactgctgctttgggatgtctggttaaagattactttccagagcctgtgactgtgagttggaattcaggcgctcttaccagcggtgttcacacattccccgcagtcttgcaatcctctggtttgtattccctgagtagcgtagtcacggtgcctagcagtagcctcgggacacaaacgtacatttgcaatgtgaatcacaaacccagtaataccaaggttgataagcgggtcgaacctaaatcctgtgacaagacccacacatgtcctccgtgcccggctcccgagctgctgggcgggccgtccgtattcctgtttccccctaaaccgaaagacactttgatgatatcacggacacccgaggttacgtgtgtggtcgtcgatgtttctcacgaggaccccgaagtcaaattcaattggtacgtggatggtgttgaggttcacaacgccaaaactaaaccccgcgaggagcagtacaattcaacctatagggttgtgagtgtccttactgtcctccatcaagattggctgaacggcaaagaatataaatgcaaagtctcaaacaaagctctgccagccccgatagaaaagaccatctccaaagctaaggggcaacccagagaacctcaagtgtacaccctccctccatcaagggaggagatgacgaaaaatcaagtaagcttgacctgtcttgtaaaagggttctacccaagtgatatagctgtagaatgggagagtaatgggcagcccgagaataattacaaaacgacaccgcccgtgctggactcagacggtagctttttcctctacagcaaacttactgtagataaaagtaggtggcagcagggaaatgttttttcctgttcagtcatgcatgaagcattgcacaaccattacacccagaagtctctcagtttgagcccgggcaagtaatgaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggaatgcttccaagtcagttgatagggtttttgctgctgtgggtgcctgcttcaagaggtattcagatgactcaatctccttcttctctgtcagccagcgtaggtgatcgcgtcaccatcacctgtcgagcctcacagtccatttctagctatctcaattggtatcagcagaagccaggaaaagcccctaaacttctcatatacgccgcctctagtctccaaagtggggtgcccagtcggttcagtggctccggatctgggactgatttcactttgactatatctagtctccagcctgaggattttgctacatattattgtcaacaatcctactctacacccctgacgttcggtggcggaacgaaggttgaaataaagaggactgtggccgccccatcagttttcattttcccgccgtctgatgaacaactcaaaagcggcacagcatctgtagtctgccttttgaacaacttttatccacgggaggcaaaagtgcaatggaaagtggataatgctctgcaaagtggtaacagccaagaaagtgtaaccgaacaggattccaaagactcaacctattccctcagttccacactcacactgtctaaggctgattacgagaagcataaggtttacgcctgtgaagttacgcaccaaggactctctagtccagttactaaaagctttaatcggggggaatgttagtgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggcgcgccgcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggcggcctcagtgagcgagcgagcgcgcagctgcctgcaggtcgacgcatgcggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaa

SEQ ID NO: 57 - protein, 453 aa, Artificial Sequence
  Description of Artificial Sequence Synthetic polypeptide
  MOD_RES (1)..(1): Any amino acid
XVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 1310 bp, Artificial Sequence
  Description of Artificial Sequence Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaa

SEQ ID NO: 60 - DNA, 16 bp, Artificial Sequence
  Description of Artificial Sequence Synthetic oligonucleotide
gcgcgctcgctcgctc

SEQ ID NO: 61 - DNA, 6 bp, Artificial Sequence
  Description of Artificial Sequence Synthetic oligonucleotide
ggttga

SEQ ID NO: 62 - DNA, 4 bp, Artificial Sequence
  Description of Artificial Sequence Synthetic oligonucleotide
agtt

SEQ ID NO: 63 - DNA, 6 bp, Artificial Sequence
  Description of Artificial Sequence Synthetic oligonucleotide
ggttgg

SEQ ID NO: 64 - DNA, 6 bp, Artificial Sequence
  Description of Artificial Sequence Synthetic oligonucleotide
agttgg

SEQ ID NO: 65 - DNA, 6 bp, Artificial Sequence
  Description of Artificial Sequence Synthetic oligonucleotide
agttga

SEQ ID NO: 66 - DNA, 6 bp, Artificial Sequence
  Description of Artificial Sequence Synthetic oligonucleotide
rrttrr

SEQ ID NO: 67 - DNA, 581 bp, Artificial Sequence
  Description of Artificial Sequence Synthetic polynucleotide
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 68 - DNA, 225 bp, Artificial Sequence
  Description of Artificial Sequence Synthetic polynucleotide
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 69 - DNA, 8 bp, Artificial Sequence
  Description of Artificial Sequence Synthetic oligonucleotide
actgaggc

SEQ ID NO: 70 - DNA, 8 bp, Artificial Sequence
  Description of Artificial Sequence Synthetic oligonucleotide
gcctcagt

SEQ ID NO: 71 - DNA, 16 bp, Artificial Sequence
  Description of Artificial Sequence Synthetic oligonucleotide
gagcgagcgagcgcgc

SEQ ID NO: 72 - DNA, 1923 bp, Artificial Sequence
  Description of Artificial Sequence Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 73 - DNA, 1272 bp, Artificial Sequence
  Description of Artificial Sequence Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 74 - DNA, 1177 bp, Artificial Sequence
  Description of Artificial Sequence Synthetic polynucleotide
ggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 75 - DNA, 547 bp, Artificial Sequence
  Description of Artificial Sequence Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 76 - DNA, 556 bp, Artificial Sequence
  Description of Artificial Sequence Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacactcgagggccctgtctcctcagcttcaggcaccaccactgacctgggacagtgaatccggacatcgattctaaggtaaatataaaatttttaagtgtataatttgttaaactactgattctaattgtttctctcttttagattccaacctttggaactga

SEQ ID NO: 77 - DNA, 1179 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 78 - DNA, 141 bp, Artificial Sequence
  Description of Artificial Sequence Synthetic polynucleotide
aataaacgataacgccgttggtggcgtgaggcatgtaaaaggttacatcattatcttgttcgccatccggttggtataaatagacgttcatgttggtttttgtttcagttgcaagttggctgcggcgcgcgcagcaccttt

SEQ ID NO: 79 - DNA, 317 bp, Artificial Sequence
  Description of Artificial Sequence Synthetic polynucleotide
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 80 - DNA, 241 bp, Artificial Sequence
  Description of Artificial Sequence Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 81 - DNA, 215 bp, Artificial Sequence
  Description of Artificial Sequence Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccac

SEQ ID NO: 82 - DNA, 546 bp, Artificial Sequence
  Description of Artificial Sequence Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 83 - DNA, 576 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 84 - DNA, 150 bp, Artificial Sequence
  Description of Artificial Sequence Synthetic polynucleotide
ataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcggccatct

SEQ ID NO: 85 - DNA, 1313 bp, Artificial Sequence
  Description of Artificial Sequence Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaa

SEQ ID NO: 86 - DNA, 213 bp, Artificial Sequence
  Description of Artificial Sequence Synthetic polynucleotide
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 87 - protein, 7 aa, Artificial Sequence
  Description of Artificial Sequence Synthetic peptide
PKKKRKV

SEQ ID NO: 88 - protein, 19 aa, Artificial Sequence
  Description of Artificial Sequence Synthetic peptide
MDWTWRILFLVAAATGAHS

SEQ ID NO: 89 - protein, 19 aa, Artificial Sequence
  Description of Artificial Sequence Synthetic peptide
MLPSQLIGFLLLWVPASRG

SEQ ID NO: 90 - protein, 7 aa, Simian virus 40
PKKKRKV

SEQ ID NO: 91 - DNA, 21 bp, Simian virus 40
cccaagaagaagaggaaggtg

SEQ ID NO: 92 - protein, 16 aa, Unknown
  Description of Unknown Nucleoplasmin bipartite NLS sequence
KRPAATKKAGQAKKKK

SEQ ID NO: 93 - protein, 9 aa, Unknown
  Description of Unknown C-myc NLS sequence
PAAKRVKLD

SEQ ID NO: 94 - protein, 11 aa, Unknown
  Description of Unknown C-myc NLS sequence
RQRRNELKRSP

SEQ ID NO: 95 - protein, 38 aa, Homo sapiens
NQSSNFGPMKGGNFGGRSSGPYGGGGQYFAKPRNQGGY

SEQ ID NO: 96 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 97 - protein, 8 aa, Unknown
  Description of Unknown Myoma T protein sequence
VSRKRPRP

SEQ ID NO: 98 - protein, 8 aa, Unknown
  Description of Unknown Myoma T protein sequence
PPKKARED

SEQ ID NO: 99 - protein, 8 aa, Homo sapiens
PQPKKKPL

SEQ ID NO: 100 - protein, 12 aa, Mus musculus
SALIKKKKKMAP

SEQ ID NO: 101 - DNA, 70 bp, Artificial Sequence
  Description of Artificial Sequence Synthetic oligonucleotide
gcgcgctcgctcgctcactgaggccgcccgggaaacccgggcgtgcgcctcagtgagcgagcgagcgcgc

SEQ ID NO: 102 - DNA, 70 bp, Artificial Sequence
  Description of Artificial Sequence Synthetic oligonucleotide
gcgcgctcgctcgctcactgaggcgcacgcccgggtttcccgggcggcctcagtgagcgagcgagcgcgc

SEQ ID NO: 103 - DNA, 72 bp, Artificial Sequence
  Description of Artificial Sequence Synthetic oligonucleotide
gcgcgctcgctcgctcactgaggccgtcgggcgacctttggtcgcccggcctcagtgagcgagcgagcgcgc

SEQ ID NO: 104 - DNA, 72 bp, Artificial Sequence
  Description of Artificial Sequence Synthetic oligonucleotide
gcgcgctcgctcgctcactgaggccgggcgaccaaaggtcgcccgacggcctcagtgagcgagcgagcgcgc

SEQ ID NO: 105 - DNA, 72 bp, Artificial Sequence
  Description of Artificial Sequence Synthetic oligonucleotide
gcgcgctcgctcgctcactgaggccgcccgggcaaagcccgggcgtcggcctcagtgagcgagcgagcgcgc

SEQ ID NO: 106 - DNA, 72 bp, Artificial Sequence
  Description of Artificial Sequence Synthetic oligonucleotide
gcgcgctcgctcgctcactgaggccgacgcccgggctttgcccgggcggcctcagtgagcgagcgagcgcgc

SEQ ID NO: 107 - DNA, 83 bp, Artificial Sequence
  Description of Artificial Sequence Synthetic oligonucleotide
gcgcgctcgctcgctcactgaggccgcccgggcaaagcccgggcgtcgggctttgcccggcctcagtgagcgagcgagcgcgc

SEQ ID NO: 108 - DNA, 83 bp, Artificial Sequence
  Description of Artificial Sequence Synthetic oligonucleotide
gcgcgctcgctcgctcactgaggccgggcaaagcccgacgcccgggctttgcccgggcggcctcagtgagcgagcgagcgcgc

SEQ ID NO: 109 - DNA, 77 bp, Artificial Sequence
  Description of Artificial Sequence Synthetic oligonucleotide
gcgcgctcgctcgctcactgaggccgaaacgtcgggcgacctttggtcgcccggcctcagtgagcgagcgagcgcgc

SEQ ID NO: 110 - DNA, 77 bp, Artificial Sequence
  Description of Artificial Sequence Synthetic oligonucleotide
gcgcgctcgctcgctcactgaggccgggcgaccaaaggtcgcccgacgtttcggcctcagtgagcgagcgagcgcgc

SEQ ID NO: 111 - DNA, 51 bp, Artificial Sequence
  Description of Artificial Sequence Synthetic oligonucleotide
gcgcgctcgctcgctcactgaggcaaagcctcagtgagcgagcgagcgcgc

SEQ ID NO: 112 - DNA, 51 bp, Artificial Sequence
  Description of Artificial Sequence Synthetic oligonucleotide
gcgcgctcgctcgctcactgaggctttgcctcagtgagcgagcgagcgcgc

SEQ ID NO: 113 - DNA, 80 bp, Artificial Sequence
  Description of Artificial Sequence Synthetic oligonucleotide
gcgcgctcgctcgctcactgaggccgcccgggcgtcgggcgacctttggtcgcccggcctcagtgagcgagcgagcgcgc

SEQ ID NO: 114 - DNA, 80 bp, Artificial Sequence
  Description of Artificial Sequence Synthetic oligonucleotide
gcgcgctcgctcgctcactgaggccgggcgaccaaaggtcgcccgacgcccgggcggcctcagtgagcgagcgagcgcgc

SEQ ID NO: 115 - DNA, 79 bp, Artificial Sequence
  Description of Artificial Sequence Synthetic oligonucleotide
gcgcgctcgctcgctcactgaggcgcccgggcgtcgggcgacctttggtcgcccggcctcagtgagcgagcgagcgcgc

SEQ ID NO: 116 - DNA, 79 bp, Artificial Sequence
  Description of Artificial Sequence Synthetic oligonucleotide
gcgcgctcgctcgctcactgaggccgggcgaccaaaggtcgcccgacgcccgggcgcctcagtgagcgagcgagcgcgc

SEQ ID NO: 117 - protein, 5 aa, Influenza virus
DRLRR

SEQ ID NO: 118 - protein, 7 aa, Influenza virus
PKQKKRK

SEQ ID NO: 119 - protein, 10 aa, Hepatitis delta virus
RKLKKKIKKL

SEQ ID NO: 120 - protein, 10 aa, Mus musculus
REKKKFLKRR

SEQ ID NO: 121 - protein, 20 aa, Homo sapiens
KRKGDEVDGVDEVAKKKSKK

SEQ ID NO: 122 - protein, 17 aa, Homo sapiens
RKCLQAGMNLEARKTKK

SEQ ID NO: 123 - DNA, 8 bp, Artificial Sequence
  Description of Artificial Sequence Synthetic oligonucleotide
gtttaaac

SEQ ID NO: 124 - DNA, 8 bp, Artificial Sequence
  Description of Artificial Sequence Synthetic oligonucleotide
ttaattaa

SEQ ID NO: 125 - DNA, 141 bp, Artificial Sequence; identical to SEQ ID NO: 78
  Description of Artificial Sequence Synthetic polynucleotide

SEQ ID NO: 126 - DNA, 317 bp, Artificial Sequence; identical to SEQ ID NO: 79
  Description of Artificial Sequence Synthetic polynucleotide

SEQ ID NO: 127 - DNA, 72 bp, Artificial Sequence; identical to SEQ ID NO: 104
  Description of Artificial Sequence Synthetic oligonucleotide

SEQ ID NO: 128 - DNA, 60 bp, Artificial Sequence
  Description of Artificial Sequence Synthetic oligonucleotide
gagacagacacactcctgctatgggtactgctgctctgggttccaggttccactggtgac

SEQ ID NO: 129 - DNA, 1260 bp, Adeno-associated virus - 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 130 - DNA, 1932 bp, Adeno-associated virus - 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 131 - DNA, 1876 bp, Artificial Sequence
  Description of Artificial Sequence Synthetic polynucleotide
cgcagccaccatggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 132 - DNA, 1194 bp, Artificial Sequence
  Description of Artificial Sequence Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 133 - DNA, 1876 bp, Artificial Sequence; identical to SEQ ID NO: 131
  Description of Artificial Sequence Synthetic polynucleotide

SEQ ID NO: 134 - DNA, 51 bp, Artificial Sequence
  Description of Artificial Sequence Synthetic oligonucleotide
ctaggccgcccgggcaaagcccgggcgtcgggcgacctttggtcgcccggc

SEQ ID NO: 135 - DNA, 65 bp, Artificial Sequence
  Description of Artificial Sequence Synthetic oligonucleotide
ctaggactgaggccgcccgggcaaagcccgggcgtcgggcgacctttggtcgcccggcctcagtc

SEQ ID NO: 136 - DNA, 67 bp, Artificial Sequence
  Description of Artificial Sequence Synthetic oligonucleotide
ggactgaggccgcccgggcaaagcccgggcgtcgggcgacctttggtcgcccggcctcagtcctgca

SEQ ID NO: 137 - DNA, 41 bp, Artificial Sequence
  Description of Artificial Sequence Synthetic oligonucleotide
gtgcgggcgaccaaaggtcgcccgacgcccgggcgcactca

SEQ ID NO: 138 - DNA, 56 bp, Artificial Sequence
  Description of Artificial Sequence Synthetic oligonucleotide
ggactgaggccgggcgaccaaaggtcgcccgacgcccgggcggcctcagtcctgca

SEQ ID NO: 139 - DNA, 54 bp, Artificial Sequence
  Description of Artificial Sequence Synthetic oligonucleotide
ctaggactgaggccgcccgggcgtcgggcgacctttggtcgcccggcctcagtc

SEQ ID NO: 140 - DNA, 48 bp, Artificial Sequence
  Description of Artificial Sequence Synthetic oligonucleotide
ggactgaggccgggcgaccaaaggtcgcccgacggcctcagtcctgca

SEQ ID NO: 141 - DNA, 46 bp, Artificial Sequence
  Description of Artificial Sequence Synthetic oligonucleotide
ctaggactgaggccgtcgggcgacctttggtcgcccggcctcagtc

SEQ ID NO: 142 - DNA, 67 bp, Artificial Sequence
  Description of Artificial Sequence Synthetic oligonucleotide
ggactgaggcccgggcgaccaaaggtcgcccgacgcccgggctttgcccgggcgcctcagtcctgca

SEQ ID NO: 143 - DNA, 47 bp, Artificial Sequence
  Description of Artificial Sequence Synthetic oligonucleotide
atacctaggcacgcgtgttactagttattaatagtaatcaattacgg

SEQ ID NO: 144 - DNA, 29 bp, Artificial Sequence
  Description of Artificial Sequence Synthetic oligonucleotide
atacctaggggccgcacgcgtgttactag

SEQ ID NO: 145 - DNA, 42 bp, Artificial Sequence
  Description of Artificial Sequence Synthetic oligonucleotide
atacactcagtgcctgcaggcacgtggtccggagatccagac

SEQ ID NO: 146 - DNA, 3754 bp, Artificial Sequence
  Description of Artificial Sequence Synthetic polynucleotide
cctaggtgagcgagcgagcgcgcagagagggagtggccaactccatcactaggggttccttgtagttaatgattaacccgccatgctacttatcgcggccgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagccagtttaaacgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcagcaggaacccctagtgatggagttggccactccctctctgcgcgctcgctcgctccctgcaggactgaggccgggcgaccaaaggtcgcccgacgcccgggcggcctcagtcctgcagg

SEQ ID NO: 147 - DNA, 8418 bp, Artificial Sequence
  Description of Artificial Sequence Synthetic polynucleotide
ggcagctgcgcgctcgctcgctcacctaggccgcccgggcaaagcccgggcgtcgggcgacctttggtcgcccggcctaggtgagcgagcgagcgcgcagagagggagtggccaactccatcactaggggttccttgtagttaatgattaacccgccatgctacttatcgcggccgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagccagtttaaacgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcagcaggaacccctagtgatggagttggccactccctctctgcgcgctcgctcgctccctgcaggactgaggccgggcgaccaaaggtcgcccgacgcccgggcggcctcagtcctgcagggagcgagcgagcgcgcagctgcctgcacgggcgcgccggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagtgagctccctgca

SEQ ID NO: 148 - DNA, 225 bp, Artificial Sequence; identical to SEQ ID NO: 68
  Description of Artificial Sequence Synthetic polynucleotide

SEQ ID NO: 149 - DNA, 1177 bp, Artificial Sequence; identical to SEQ ID NO: 74
  Description of Artificial Sequence Synthetic polynucleotide

SEQ ID NO: 150 - DNA, 1326 bp, Artificial Sequence
  Description of Artificial Sequence Synthetic polynucleotide
ctgcagggcccactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaa

SEQ ID NO: 151 - DNA, 573 bp, Mus musculus
gtaagagttttatgttttttcatctctgcttgtatttttctagtaatggaagcctggtattttaaaatagttaaattttcctttagtgctgatttctagattattattactgttgttgttgttattattgtcattatttgcatctgagaacccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgt

SEQ ID NO: 152 - DNA, 1993 bp, Mus musculus
acgatcgggaactggcatcttcagggagtagcttaggtcagtgaagagaagaacaaaaagcagcatattacagttagttgtcttcatcaatctttaaatatgttgtgtggtttttctctccctgtttccacagacaagagtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagatctcatggtatcttgagaacattattttgtctctttgtagtactgaaaccttatacatgtgaagtaaggggtctatacttaagtcacatctccaaccttagtaatgttttaatgtagtaaaaaaatgagtaattaatttatttttagaaggtcaatagtatcatgtattccaaataacagaggtatatggttagaaaagaaacaattcaaaggacttatataatatctagccttgacaatgaataaatttagagagtagtttgcctgtttgcctcatgttcataaatctattgacacatatgtgcatctgcacttcagcatggtagaagtccatattcctttgcttggaaaggcaggtgttcccattacgcctcagagaatagctgacgggaagaggctttctagatagttgtatgaaagatatacaaaatctcgcaggtatacacaggcatgatttgctggttgggagagccacttgcctcatactgaggtttttgtgtctgcttttcagagtcctgattgccttttcccagtatctccagaaatgctcatacgatgagcatgccaaattagtgcaggaagtaacagactttgcaaagacgtgtgttgccgatgagtctgccgccaactgtgacaaatcccttgtgagtaccttctgattttgtggatctactttcctgctttctggaactctgtttcaaagccaatcatgactccatcacttaaggccccgggaacactgtggcagagggcagcagagagattgataaagccagggtgatgggaattttctgtgggactccatttcatagtaattgcagaagctacaatacactcaaaaagtctcaccacatgactgcccaaatgggagcttgacagtgacagtgacagtagatatgccaaagtggatgagggaaagaccacaagagctaaaccctgtaaaaagaactgtaggcaactaaggaatgcagagagaagaagttgccttggaagagcataccaactgcctctccaataccaatggtcatccctaaaacatacgtatgaataacatgcagactaagcaggctacatttaggaatatacatgtatttacataaatgtatatgcatgtaacaacaatgaatgaaaactgaggtcatggatctgaaagagagcaagggggcttacatgagagggtttggagggaggggttggagggagggaggtattattctttagttttacagggaacgtagtaaaaacataggcttctcccaaaggagcagagcccatgaggagctgtgcaaggttccccagcttgattttacctgctcctcaaattcccttgatttgtttttattataatgactttactcctagcttttagtgtcagatagaaaacatggaagg

SEQ ID NO: 153 - DNA, 1350 bp, Homo sapiens
taggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttatttt

SEQ ID NO: 154 - DNA, 1223 bp, Homo sapiens
tgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactg

SEQ ID NO: 155 - DNA, 215 bp, Artificial Sequence
  Description of Artificial Sequence Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 156 - DNA, 141 bp, Artificial Sequence
  Description of Artificial Sequence Synthetic polynucleotide
cctgcaggcagctgcgcgctcgctcgctcacctaggccgcccgggcaaagcccgggcgtcgggcgacctttggtcgcccggcctaggtgagcgagcgagcgcgcagagagggagtggccaactccatcactaggggttcct

SEQ ID NO: 157 - DNA, 19 bp, Artificial Sequence
  Description of Artificial Sequence Synthetic oligonucleotide
gcgcgctcgctcgctcacc

SEQ ID NO: 158 - DNA, 22 bp, Artificial Sequence
  Description of Artificial Sequence Synthetic oligonucleotide
ctaggtgagcgagcgagcgcgc

SEQ ID NO: 159 - DNA, 75 bp, Artificial Sequence
  Description of Artificial Sequence Synthetic oligonucleotide
cctgcaggactgaggccgcccgggcaaagcccgggcgtcgggcgacctttggtcgcccggcctcagtcctgcagg

SEQ ID NO: 160 - DNA, 130 bp, Artificial Sequence
  Description of Artificial Sequence Synthetic polynucleotide
aggaacccctagtgatggagttggccactccctctctgcgcgctcgctcgctcactgagtgcgggcgaccaaaggtcgcccgacgcccgggcgcactcagtgagcgagcgagcgcgcagctgcctgcagg

SEQ ID NO: 161 - DNA, 142 bp, Artificial Sequence
  Description of Artificial Sequence Synthetic polynucleotide
cctgcaggcagctgcgcgctcgctcgctccctaggactgaggccgcccgggcgtcgggcgacctttggtcgcccggcctcagtcctagggagcgagcgagcgcgcagagagggagtggccaactccatcactaggggttcct

SEQ ID NO: 162 - DNA, 80 bp, Artificial Sequence
  Description of Artificial Sequence Synthetic oligonucleotide
gcgcgctcgctcgctcactgagtgcgggcgaccaaaggtcgcccgacgcccgggcgcactcagtgagcgagcgagcgcgc

SEQ ID NO: 163 - DNA, 21 bp, Artificial Sequence
  Description of Artificial Sequence Synthetic oligonucleotide
gcgcgctcgctcgctcactga

SEQ ID NO: 164 - DNA, 18 bp, Artificial Sequence
  Description of Artificial Sequence Synthetic oligonucleotide
gtgagcgagcgagcgcgc

SEQ ID NO: 165 - DNA, 89 bp, Artificial Sequence
  Description of Artificial Sequence Synthetic oligonucleotide
gcgcgctcgctcgctcactgaggccgcccgggcaaagcccgggcgtcgggcgactttgtcgcccggcctcagtgagcgagcgagcgcgc

SEQ ID NO: 166 - DNA, 89 bp, Artificial Sequence
  Description of Artificial Sequence Synthetic oligonucleotide
gcgcgctcgctcgctcactgaggccgggcgacaaagtcgcccgacgcccgggctttgcccgggcggcctcagtgagcgagcgagcgcgc

SEQ ID NO: 167 - DNA, 87 bp, Artificial Sequence
  Description of Artificial Sequence Synthetic oligonucleotide
gcgcgctcgctcgctcactgaggccgcccgggcaaagcccgggcgtcgggcgattttcgcccggcctcagtgagcgagcgagcgcgc

SEQ ID NO: 168 - DNA, 87 bp, Artificial Sequence
  Description of Artificial Sequence Synthetic oligonucleotide
gcgcgctcgctcgctcactgaggccgggcgaaaatcgcccgacgcccgggctttgcccgggcggcctcagtgagcgagcgagcgcgc

SEQ ID NO: 169 - DNA, 85 bp, Artificial Sequence
  Description of Artificial Sequence Synthetic oligonucleotide
gcgcgctcgctcgctcactgaggccgcccgggcaaagcccgggcgtcgggcgtttcgcccggcctcagtgagcgagcgagcgcgc

SEQ ID NO: 170 - DNA, 85 bp, Artificial Sequence
  Description of Artificial Sequence Synthetic oligonucleotide
gcgcgctcgctcgctcactgaggccgggcgaaacgcccgacgcccgggctttgcccgggcggcctcagtgagcgagcgagcgcgc

SEQ ID NO: 171 - DNA, 89 bp, Artificial Sequence
  Description of Artificial Sequence Synthetic oligonucleotide
gcgcgctcgctcgctcactgaggccgcccgggaaacccgggcgtcgggcgacctttggtcgcccggcctcagtgagcgagcgagcgcgc

SEQ ID NO: 172 - DNA, 89 bp, Artificial Sequence
  Description of Artificial Sequence Synthetic oligonucleotide
gcgcgctcgctcgctcactgaggccgggcgaccaaaggtcgcccgacgcccgggtttcccgggcggcctcagtgagcgagcgagcgcgc

SEQ ID NO: 173 - DNA, 87 bp, Artificial Sequence
  Description of Artificial Sequence Synthetic oligonucleotide
gcgcgctcgctcgctcactgaggccgcccggaaaccgggcgtcgggcgacctttggtcgcccggcctcagtgagcgagcgagcgcgc

SEQ ID NO: 174 - DNA, 87 bp, Artificial Sequence
  Description of Artificial Sequence Synthetic oligonucleotide
gcgcgctcgctcgctcactgaggccgggcgaccaaaggtcgcccgacgcccggtttccgggcggcctcagtgagcgagcgagcgcgc

SEQ ID NO: 175 - DNA, 85 bp, Artificial Sequence
  Description of Artificial Sequence Synthetic oligonucleotide
gcgcgctcgctcgctcactgaggccgcccgaaacgggcgtcgggcgacctttggtcgcccggcctcagtgagcgagcgagcgcgc

SEQ ID NO: 176 - DNA, 85 bp, Artificial Sequence
  Description of Artificial Sequence Synthetic oligonucleotide
gcgcgctcgctcgctcactgaggccgggcgaccaaaggtcgcccgacgcccgtttcgggcggcctcagtgagcgagcgagcgcgc

SEQ ID NO: 177 - DNA, 83 bp, Artificial Sequence
  Description of Artificial Sequence Synthetic oligonucleotide
gcgcgctcgctcgctcactgaggccgcccaaagggcgtcgggcgacctttggtcgcccggcctcagtgagcgagcgagcgcgc

SEQ ID NO: 178 - DNA, 83 bp, Artificial Sequence
  Description of Artificial Sequence Synthetic oligonucleotide
gcgcgctcgctcgctcactgaggccgggcgaccaaaggtcgcccgacgccctttgggcggcctcagtgagcgagcgagcgcgc

SEQ ID NO: 179 - DNA, 81 bp, Artificial Sequence
  Description of Artificial Sequence Synthetic oligonucleotide
gcgcgctcgctcgctcactgaggccgccaaaggcgtcgggcgacctttggtcgcccggcctcagtgagcgagcgagcgcgc

SEQ ID NO: 180 - DNA, 81 bp, Artificial Sequence
  Description of Artificial Sequence Synthetic oligonucleotide
gcgcgctcgctcgctcactgaggccgggcgaccaaaggtcgcccgacgcctttggcggcctcagtgagcgagcgagcgcgc

SEQ ID NO: 181 - DNA, 79 bp, Artificial Sequence
  Description of Artificial Sequence Synthetic oligonucleotide
gcgcgctcgctcgctcactgaggccgcaaagcgtcgggcgacctttggtcgcccggcctcagtgagcgagcgagcgcgc

SEQ ID NO: 182 - DNA, 79 bp, Artificial Sequence
  Description of Artificial Sequence Synthetic oligonucleotide
gcgcgctcgctcgctcactgaggccgggcgaccaaaggtcgcccgacgctttgcggcctcagtgagcgagcgagcgcgc

SEQ ID NO: 183 - DNA, 81 bp, Artificial Sequence
  Description of Artificial Sequence Synthetic oligonucleotide
ctgcgcgctcgctcgctcactgaggccgaaacgtcgggcgacctttggtcgcccggcctcagtgagcgagcgagcgcgcag

SEQ ID NO: 184 - DNA, 81 bp, Artificial Sequence
  Description of Artificial Sequence Synthetic oligonucleotide
ctgcgcgctcgctcgctcactgaggccgggcgaccaaaggtcgcccgacgtttcggcctcagtgagcgagcgagcgcgcag

SEQ ID NO: 185 - DNA, 72 bp, Artificial Sequence; identical to SEQ ID NO: 104
  Description of Artificial Sequence Synthetic oligonucleotide

SEQ ID NO: 186 - DNA, 80 bp, Artificial Sequence; identical to SEQ ID NO: 114
  Description of Artificial Sequence Synthetic oligonucleotide

SEQ ID NO: 187 - DNA, 79 bp, Artificial Sequence; identical to SEQ ID NO: 115
  Description of Artificial Sequence Synthetic oligonucleotide

SEQ ID NO: 188 - DNA, 48 bp, Artificial Sequence
  Description of Artificial Sequence Synthetic oligonucleotide
ggagtcaaagttctgtttgccctgatctgcatcgctgtggccgaggcc

SEQ ID NO: 189 - DNA, 99 bp, Artificial Sequence
  Description of Artificial Sequence Synthetic oligonucleotide
attcataccaacttgaagaaaaagttcagcctcttcatcctggtctttctcctgttcgcagtcatctgtgtttggaagaaagggagcgactatgaggcc

SEQ ID NO: 190 - DNA, 588 bp, Artificial Sequence
  Description of Artificial Sequence Synthetic polynucleotide
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc